SEQUENCE LISTING (54 sequences)

SEQ ID NO: 1 - protein, 30 aa, synthetic construct
  REGION 1..30: note = Human Insulin B-Chain
FVNQHLCGSHLVEALYLVCGERGFFYTPKT

SEQ ID NO: 2 - protein, 21 aa, synthetic construct
  REGION 1..21: note = Human Insulin A-Chain
GIVEQCCTSICSLYQLENYCN

SEQ ID NO: 3 - protein, 86 aa, synthetic construct
  REGION 1..86: note = Human ProInsulin
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 4 - protein, 30 aa, synthetic construct
  REGION 1..30: note = Insulin B-chain Analog
FVNQHLCGSDLVEALALVCGERGFFYTDPT

SEQ ID NO: 5 - protein, 30 aa, synthetic construct
  REGION 1..30: note = Insulin B-chain Analog
FVNQHLCGSDLVEALYLVCGERGFFYTDPT

SEQ ID NO: 6 - protein, 31 aa, synthetic construct
  REGION 1..31: note = Insulin B-chain Analog
  VARIANT 28..29: note = XX is PR, PE, PA, DP, EP, AP, DK, EK, ER, AK, or AR
FVNQHLCGSHLVEALYLVCGERGFFYTXXPT

SEQ ID NO: 7 - protein, 30 aa, synthetic construct
  REGION 1..30: note = Insulin B-chain Analog
FVNQHLCGSHLVEALYLVCGERGFFYTKPT

SEQ ID NO: 8 - protein, 6 aa, synthetic construct
  REGION 1..6: note = C-Chain
GGGPRR

SEQ ID NO: 9-12 - skipped (4 entries)

SEQ ID NO: 13 - protein, 21 aa, synthetic construct
  REGION 1..21: note = Insulin A-chain Analog
  VARIANT 8: note = X is T, H, S, E or A
  VARIANT 14: note = X is any amino acid other than P
GIVEQCCXSICSLXQLENYCN

SEQ ID NO: 14 - protein, 21 aa, synthetic construct
  REGION 1..21: note = Insulin A-chain Analog
GIVEQCCHSICSLYQLENYCN

SEQ ID NO: 15 - protein, 57 aa, synthetic construct
  REGION 1..57: note = Insulin Polypeptide
FVNQHLCGSDLVEALYLVCGERGFFYTDPTGGGPRRGIVEQCCHSICSLYQLENYCN

SEQ ID NO: 16 - protein, 57 aa, synthetic construct
  REGION 1..57: note = Insulin Polypeptide
FVNQHLCGSDLVEALALVCGERGFFYTDPTGGGPRRGIVEQCCHSICSLYQLENYCN

SEQ ID NO: 17 - protein, 57 aa, synthetic construct
  REGION 1..57: note = Insulin Polypeptide
FVNQHLCGSHLVEALYLVCGERGFFYTDPTEEGPRRGIVEQCCHSICSLEQLENYCN

SEQ ID NO: 18 - protein, 58 aa, synthetic construct
  REGION 1..58: note = Insulin Polypeptide
FVNQHLCGSHLVEALYLVCGERGFFYTDPTEEGAPRRGIVEQCCHSICSLEQLENYCN

SEQ ID NO: 19 - protein, 59 aa, synthetic construct
  REGION 1..59: note = Insulin Polypeptide
FVNQHLCGSHLVEALYLVCGERGFFYTDPTEEGAAPRRGIVEQCCHSICSLEQLENYCN

SEQ ID NO: 20 - protein, 60 aa, synthetic construct
  REGION 1..60: note = Insulin Polypeptide
FVNQHLCGSHLVEALYLVCGERGFFYTDPTEEGAAAPRRGIVEQCCHSICSLEQLENYCN

SEQ ID NO: 21 - protein, 57 aa, synthetic construct
  REGION 1..57: note = Insulin Polypeptide
FVNQHLCGSHLVEALELVCGERGFHYGGGGGGSGGGGGIVEQCCTSTCSLDQLENYC

SEQ ID NO: 22 - protein, 58 aa, synthetic construct
  REGION 1..58: note = Insulin Polypeptide
FVNQHLCGSHLVEALYLVCGERGFFYTPKAAAAAAAKGIVEQCCTSICSLYQLENYCN

SEQ ID NO: 23 - protein, 57 aa, synthetic construct
  REGION 1..57: note = Insulin Polypeptide
FVNQHLCGSHLVEALYLVCGERGFFYTPKAGGGPRRGIVEQCCTSICSLYQLENYCN

SEQ ID NO: 24 - protein, 57 aa, synthetic construct
  REGION 1..57: note = Insulin Polypeptide
FVNQHLCGSHLVEALALVCGERGFFYTDPTGGGPRRGIVEQCCTSICSLYQLENYCN

SEQ ID NO: 25 - protein, 9 aa, synthetic construct
  REGION 1..9: note = Linker
GGGGAGGGG

SEQ ID NO: 26 - protein, 9 aa, synthetic construct
  REGION 1..9: note = Linker
GGGGSGGGG

SEQ ID NO: 27 - protein, 21 aa, synthetic construct
  REGION 1..21: note = Linker
GGGGGQGGGGQGGGGQGGGGG

SEQ ID NO: 28 - protein, 228 aa, synthetic construct
  REGION 1..228: note = FC Fragment
RCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPG

SEQ ID NO: 29 - protein, 224 aa, synthetic construct
  REGION 1..224: note = FC Fragment
DCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 30 - protein, 225 aa, synthetic construct
  REGION 1..225: note = FC Fragment
CNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPG

SEQ ID NO: 31 - protein, 225 aa, synthetic construct
  REGION 1..225: note = FC Fragment
CISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPG

SEQ ID NO: 32 - protein, 223 aa, synthetic construct
  REGION 1..223: note = FC Fragment
CPKCPAPEMLGGPSVFIFPPKPKDTLLIARTPEVTCVVVDLDPEDPEVQISWFVDGKQMQTAKTQPREEQFKGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 33 - protein, 294 aa, synthetic construct
  REGION 1..294: note = Insulin-Fc Fusion Protein
FVNQHLCGSDLVEALALVCGERGFFYTDPTGGGPRRGIVEQCCHSICSLYQLENYCNGGGGAGGGGRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPG

SEQ ID NO: 34 - DNA, 942 bp, synthetic construct
  misc_feature 1..942: note = cDNA for Insulin-Fc Fusion Protein
  source 1..942: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggacgctgcactgacacccctccatgccctgtgcccgagcccctgggtggccccagcgtactgatcttcccaccgaaacccaaggacatcctgaggatcacccgcaccccggaggtgacctgcgtggtgctggacctgggcagggaggaccccgaagtgcaaatcagctggttcgtggacggaaaggaggtgcacaccgccaagacccaatcaagggagcagcagttcaacggcacctacagggtggtgagcgtgttgcccatagagcaccaggactggctgaccggcaaggagttcaagtgccgcgtgaaccacattgatctccccagccccatcgagaggactatctccaaggcccgagggagggcccacaagcccagtgtatacgtgctgccgccctctccgaaggaactgagctctagcgacaccgtgagcatcacctgcctgatcaaggacttctaccctcccgacatagacgtagagtggcagagcaacggccagcaggagcccgaaaggaagcacaggatgaccccaccccaactggacgaggacggctcatactttctttatagcaagctgagtgtggacaagagcaggtggcagcagggcgaccctttcacttgcgccgtaatgcacgagaccctgcagaatcactacaccgacctgtcactgagccatagccccggatag

SEQ ID NO: 35 - protein, 294 aa, synthetic construct
  REGION 1..294: note = Insulin-Fc Fusion Protein
FVNQHLCGSDLVEALYLVCGERGFFYTDPTGGGPRRGIVEQCCHSICSLYQLENYCNGGGGSGGGGRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPG

SEQ ID NO: 36 - DNA, 942 bp, synthetic construct
  misc_feature 1..942: note = cDNA for Insulin-Fc Fusion Protein
  source 1..942: mol_type = other DNA
atggaatggagctgggtctttctcttcttcctgtcagtaacgactggtgtccactccttcgtgaaccagcacctgtgcggctccgacctggtggaagctctgtatctcgtgtgcggcgagcggggcttcttctacaccgatcccactggaggcggtccacgcagaggcatcgtggaacagtgctgccactccatctgctccctgtaccagctggaaaactactgcaatggcggaggtggttcaggaggcggtggacgctgcactgacacccctccatgccctgtgcccgagcccctgggtggccccagcgtactgatcttcccaccgaaacccaaggacatcctgaggatcacccgcaccccggaggtgacctgcgtggtgctggacctgggcagggaggaccccgaagtgcaaatcagctggttcgtggacggaaaggaggtgcacaccgccaagacccaatcaagggagcagcagttcaacggcacctacagggtggtgagcgtgttgcccatagagcaccaggactggctgaccggcaaggagttcaagtgccgcgtgaaccacattgatctccccagccccatcgagaggactatctccaaggcccgagggagggcccacaagcccagtgtatacgtgctgccgccctctccgaaggaactgagctctagcgacaccgtgagcatcacctgcctgatcaaggacttctaccctcccgacatagacgtagagtggcagagcaacggccagcaggagcccgaaaggaagcacaggatgaccccaccccaactggacgaggacggctcatactttctttatagcaagctgagtgtggacaagagcaggtggcagcagggcgaccctttcacttgcgccgtaatgcacgagaccctgcagaatcactacaccgacctgtcactgagccatagccccggatag

SEQ ID NO: 37 - protein, 294 aa, synthetic construct
  REGION 1..294: note = Insulin-Fc Fusion Protein
FVNQHLCGSHLVEALYLVCGERGFFYTPKAGGGPRRGIVEQCCTSICSLYQLENYCNGGGGSGGGGRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPG

SEQ ID NO: 38 - DNA, 942 bp, synthetic construct
  misc_feature 1..942: note = cDNA for Insulin-Fc Fusion Protein
  source 1..942: mol_type = other DNA
atggaatggagctgggtctttctcttcttcctgtcagtaacgactggtgtccactccttcgtgaaccagcacctgtgcggctcccacctggtggaagctctgtatctcgtgtgcggcgagcggggcttcttctacacccccaaggccggaggtggacctcgacggggcatcgtggaacagtgctgcacctccatctgctccctgtaccagctggaaaactactgcaatggcggaggtggttcaggaggcggtggacgctgcactgacacccctccatgccctgtgcccgagcccctgggtggccccagcgtactgatcttcccaccgaaacccaaggacatcctgaggatcacccgcaccccggaggtgacctgcgtggtgctggacctgggcagggaggaccccgaagtgcaaatcagctggttcgtggacggaaaggaggtgcacaccgccaagacccaatcaagggagcagcagttcaacggcacctacagggtggtgagcgtgttgcccatagagcaccaggactggctgaccggcaaggagttcaagtgccgcgtgaaccacattgatctccccagccccatcgagaggactatctccaaggcccgagggagggcccacaagcccagtgtatacgtgctgccgccctctccgaaggaactgagctctagcgacaccgtgagcatcacctgcctgatcaaggacttctaccctcccgacatagacgtagagtggcagagcaacggccagcaggagcccgaaaggaagcacaggatgaccccaccccaactggacgaggacggctcatactttctttatagcaagctgagtgtggacaagagcaggtggcagcagggcgaccctttcacttgcgccgtaatgcacgagaccctgcagaatcactacaccgacctgtcactgagccatagccccggatag

SEQ ID NO: 39 - protein, 295 aa, synthetic construct
  REGION 1..295: note = Insulin-Fc Fusion Protein
FVNQHLCGSHLVEALYLVCGERGFFYTPKAAAAAAAKGIVEQCCTSICSLYQLENYCNGGGGSGGGGRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPG

SEQ ID NO: 40 - DNA, 945 bp, synthetic construct
  misc_feature 1..945: note = cDNA for Insulin-Fc Fusion Protein
  source 1..945: mol_type = other DNA
atggaatggagctgggtctttctcttcttcctgtcagtaacgactggtgtccactccttcgtgaaccagcacctgtgcggctcccacctggtggaagctctgtatctcgtgtgcggcgagcggggcttcttctacacccccaaggctgccgcagcagctgccgctaaaggcatcgtggaacagtgctgcacctccatctgctccctgtaccagctggaaaactactgcaatggcggaggtggttcaggaggcggtggacgctgcactgacacccctccatgccctgtgcccgagcccctgggtggccccagcgtactgatcttcccaccgaaacccaaggacatcctgaggatcacccgcaccccggaggtgacctgcgtggtgctggacctgggcagggaggaccccgaagtgcaaatcagctggttcgtggacggaaaggaggtgcacaccgccaagacccaatcaagggagcagcagttcaacggcacctacagggtggtgagcgtgttgcccatagagcaccaggactggctgaccggcaaggagttcaagtgccgcgtgaaccacattgatctccccagccccatcgagaggactatctccaaggcccgagggagggcccacaagcccagtgtatacgtgctgccgccctctccgaaggaactgagctctagcgacaccgtgagcatcacctgcctgatcaaggacttctaccctcccgacatagacgtagagtggcagagcaacggccagcaggagcccgaaaggaagcacaggatgaccccaccccaactggacgaggacggctcatactttctttatagcaagctgagtgtggacaagagcaggtggcagcagggcgaccctttcacttgcgccgtaatgcacgagaccctgcagaatcactacaccgacctgtcactgagccatagccccggatag

SEQ ID NO: 41 - protein, 290 aa, synthetic construct
  REGION 1..290: note = Insulin-FC Fusion Protein
FVNQHLCGSDLVEALALVCGERGFFYTDPTGGGPRRGIVEQCCHSICSLYQLENYCNGGGGAGGGGDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 42 - DNA, 930 bp, synthetic construct
  misc_feature 1..930: note = cDNA for Insulin-Fc Fusion Protein
  source 1..930: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggagactgccccaagtgccccgctcccgagatgctgggcggacccagcgtgttcatcttccctcccaagcccaaggacacactgctgatcgccaggaccccggaggtgacctgcgtggtggtggacctggatcccgaagaccccgaggtgcagatcagctggttcgtggatggaaagcagatgcagaccgccaagacccaaccccgggaagagcagttcaacggcacctacagggtggtgagtgtgttgcccatcggccaccaggactggctgaaggggaagcaattcacatgcaaggttaataacaaggccctgcccagccccatcgagaggaccatcagcaaggccaggggccaggcccaccagccatctgtgtacgtgctgcccccatctagggaggaactgagcaagaacacagtcagccttacttgcctgatcaaggacttcttcccaccggacatagacgtggagtggcagagtaacggccagcaggagcccgagagcaagtataggaccacaccgccccaactggacgaggacggaagctacttcctctacagcaaattgagcgttgacaaaagcaggtggcagcgaggcgacaccttcatctgcgccgtgatgcacgaggctttgcataaccactacacccaggagagcctgtcccacagccccggatag

SEQ ID NO: 43 - protein, 291 aa, synthetic construct
  REGION 1..291: note = Insulin-Fc Fusion Protein
FVNQHLCGSDLVEALALVCGERGFFYTDPTGGGPRRGIVEQCCHSICSLYQLENYCNGGGGAGGGGCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPG

SEQ ID NO: 44 - DNA, 933 bp, synthetic construct
  misc_feature 1..933: note = cDNA for Insulin-Fc Fusion Protein
  source 1..933: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggatgcaacaactgcccgtgtccgggatgcggcctcctgggcggaccgagcgtgttcattttccctcctaagcccaaggacattctggtgaccgccaggacccccacggtgacctgcgtggtagtagatctcgatcccgaaaacccagaggtgcaaatcagctggttcgtggactctaagcaagtgcaaaccgccaacacgcaaccccgcgaggaacagagcaacggcacctacagggtggtgagcgtgctgcccatcgggcatcaggactggctgagcggcaagcagtttaaatgcaaggttaacaacaaggcactgcccagccccatcgaggagatcatcagcaagaccccgggacaggcccaccagcccaacgtgtacgtccttcctccgagccgcgacgagatgagcaagaacaccgtgacgctgacctgtttggtgaaggacttcttcccacccgagatcgacgtggagtggcaaagcaatggccagcaggagcccgagagcaaatacaggatgaccccaccccaactggatgaggatggcagctatttcctctacagcaaattgtccgtggacaaaagcaggtggcagaggggcgacaccttcatctgcgccgtcatgcacgaggcccttcacaatcactacacccagatcagcctgagccactctcccggatag

SEQ ID NO: 45 - protein, 291 aa, synthetic construct
  REGION 1..291: note = Insulin-Fc Fusion Protein
FVNQHLCGSDLVEALALVCGERGFFYTDPTGGGPRRGIVEQCCHSICSLYQLENYCNGGGGAGGGGCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPG

SEQ ID NO: 46 - DNA, 933 bp, synthetic construct
  misc_feature 1..933: note = cDNA for Insulin-Fc Fusion Protein
  source 1..933: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggatgcatcagcccctgccccgtgccggagagcctgggtggccctagcgtgttcatattccctcccaagcccaaggacatcctgaggatcaccaggacccccgagatcacctgtgtggtgctggatcttggcagggaagaccccgaagtccagatcagctggttcgtggatggcaaggaggtgcacaccgccaagacccagccgagggagcagcagttcaactccacctacagggtggtgagcgtgctgcctatcgagcatcaggactggctgaccggcaaagagttcaagtgcagggtgaaccacatcggcctgcccagccccatcgagaggaccatcagcaaagccaggggccaggcccaccagcccagtgtgtacgtgcttccccctagcccaaaggaactgagtagcagcgataccgtgaccctgacctgcctgatcaaggactttttcccgccagaaatagacgtggagtggcagagcaacggccagccggagcccgagagcaaataccacaccaccgcccctcaactggacgaggacgggagctacttcctgtatagcaagctgagcgttgacaagagcaggtggcaacagggcgacaccttcacctgcgccgtgatgcacgaagctctgcaaaaccactacaccgacctgtcactgagccatagccccggatag

SEQ ID NO: 47 - protein, 290 aa, synthetic construct
  REGION 1..290: note = Insulin-Fc Fusion Protein
FVNQHLCGSDLVEALALVCGERGFFYTDPTGGGPRRGIVEQCCHSICSLYQLENYCNGGGGAGGGGDCPKCPAPEMLGGPSVFIFPPKPKDTLLIARTPEVTCVVVDLDPEDPEVQISWFVDGKQMQTAKTQPREEQFSGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 48 - DNA, 930 bp, synthetic construct
  misc_feature 1..930: note = cDNA for Insulin-Fc Fusion Protein
  source 1..930: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggagactgccccaagtgccccgctcccgagatgctgggcggacccagcgtgttcatcttccctcccaagcccaaggacacactgctgatcgccaggaccccggaggtgacctgcgtggtggtggacctggatcccgaagaccccgaggtgcagatcagctggttcgtggatggaaagcagatgcagaccgccaagacccaaccccgggaagagcagttcagcggcacctacagggtggtgagtgtgttgcccatcggccaccaggactggctgaaggggaagcaattcacatgcaaggttaataacaaggccctgcccagccccatcgagaggaccatcagcaaggccaggggccaggcccaccagccatctgtgtacgtgctgcccccatctagggaggaactgagcaagaacacagtcagccttacttgcctgatcaaggacttcttcccaccggacatagacgtggagtggcagagtaacggccagcaggagcccgagagcaagtataggaccacaccgccccaactggacgaggacggaagctacttcctctacagcaaattgagcgttgacaaaagcaggtggcagcgaggcgacaccttcatctgcgccgtgatgcacgaggctttgcataaccactacacccaggagagcctgtcccacagccccggatag

SEQ ID NO: 49 - protein, 290 aa, synthetic construct
  REGION 1..290: note = Insulin-Fc Fusion Protein
FVNQHLCGSHLVEALALVCGERGFFYTDPTGGGPRRGIVEQCCTSICSLYQLENYCNGGGGAGGGGDCPKCPAPEMLGGPSVFIFPPKPKDTLLIARTPEVTCVVVDLDPEDPEVQISWFVDGKQMQTAKTQPREEQFQGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 50 - DNA, 930 bp, synthetic construct
  misc_feature 1..930: note = cDNA for Insulin-Fc Fusion Protein
  source 1..930: mol_type = other DNA
atggaatggagctgggtctttctcttcttcctgtcagtaacgactggtgtccactccttcgtgaaccagcacctgtgcggctcccacctggtggaagctctggctctcgtgtgcggcgagcggggcttcttctacaccgatcccactggaggcggtccacgcagaggcatcgtggaacagtgctgcacctccatctgctccctgtaccagctggaaaactactgcaatggcggaggtggtgcaggaggcggtggagactgccccaagtgccccgctcccgagatgctgggcggacccagcgtgttcatcttccctcccaagcccaaggacacactgctgatcgccaggaccccggaggtgacctgcgtggtggtggacctggatcccgaagaccccgaggtgcagatcagctggttcgtggatggaaagcagatgcagaccgccaagacccaaccccgggaagagcagttccaaggcacctacagggtggtgagtgtgttgcccatcggccaccaggactggctgaaggggaagcaattcacatgcaaggttaataacaaggccctgcccagccccatcgagaggaccatcagcaaggccaggggccaggcccaccagccatctgtgtacgtgctgcccccatctagggaggaactgagcaagaacacagtcagccttacttgcctgatcaaggacttcttcccaccggacatagacgtggagtggcagagtaacggccagcaggagcccgagagcaagtataggaccacaccgccccaactggacgaggacggaagctacttcctctacagcaaattgagcgttgacaaaagcaggtggcagcgaggcgacaccttcatctgcgccgtgatgcacgaggctttgcataaccactacacccaggagagcctgtcccacagccccggatag

SEQ ID NO: 51 - protein, 289 aa, synthetic construct
  REGION 1..289: note = Insulin-Fc Fusion Protein
FVNQHLCGSDLVEALALVCGERGFFYTDPTGGGPRRGIVEQCCHSICSLYQLENYCNGGGGAGGGGCPKCPAPEMLGGPSVFIFPPKPKDTLLIARTPEVTCVVVDLDPEDPEVQISWFVDGKQMQTAKTQPREEQFKGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 52 - DNA, 927 bp, synthetic construct
  misc_feature 1..927: note = cDNA for Insulin-Fc Fusion Protein
  source 1..927: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggatgccccaagtgccccgctcccgagatgctgggcggacccagcgtgttcatcttccctcccaagcccaaggacacactgctgatcgccaggaccccggaggtgacctgcgtggtggtggacctggatcccgaagaccccgaggtgcagatcagctggttcgtggatggaaagcagatgcagaccgccaagacccaaccccgggaagagcagttcaaaggcacctacagggtggtgagtgtgttgcccatcggccaccaggactggctgaaggggaagcaattcacatgcaaggttaataacaaggccctgcccagccccatcgagaggaccatcagcaaggccaggggccaggcccaccagccatctgtgtacgtgctgcccccatctagggaggaactgagcaagaacacagtcagccttacttgcctgatcaaggacttcttcccaccggacatagacgtggagtggcagagtaacggccagcaggagcccgagagcaagtataggaccacaccgccccaactggacgaggacggaagctacttcctctacagcaaattgagcgttgacaaaagcaggtggcagcgaggcgacaccttcatctgcgccgtgatgcacgaggctttgcataaccactacacccaggagagcctgtcccacagccccggatag

SEQ ID NO: 53 - DNA, 57 bp, synthetic construct
  misc_feature 1..57: note = Leader Sequence cDNA
  source 1..57: mol_type = other DNA
atggaatggagctgggtctttctcttcttcctgtcagtaacgactggtgtccactcc

SEQ ID NO: 54 - protein, 19 aa, synthetic construct
  REGION 1..19: note = Leader Sequence
MEWSWVFLFFLSVTTGVHS

(19) United States
(12) Patent Application Publication
(10) Pub. No.: US 2024/0182539 A1
(43) Pub. Date: Jun. 6, 2024

(54) ULTRA-LONG ACTING INSULIN-FC FUSION PROTEINS AND METHODS OF USE

(71) Applicant: Akston Biosciences Corporation, Beverly, MA (US)

(72) Inventors: Thomas M. Lancaster, Wenham, MA (US); Todd C. Zion, Salem, MA (US)

(21) Appl. No.: 18/581,048

(22) Filed: Feb. 19, 2024

Related U.S. Application Data
(63) Continuation of application No. 17/523,133, filed on Nov. 10, 2021, now Pat. No. 11,919,935.
Application No. 17/523,133 is a continuation of application No. 17/135,333, filed on Dec. 28, 2020, now Pat. No. 11,186,623.
(60) Provisional application No. 62/953,470, filed on Dec. 24, 2019.

(51) Int. Cl. C07K 14/62; C07H 21/04; C12N 5/16

(52) U.S. Cl. CPC C07K 14/62; C07H 21/04; C07K 2319/30; C07K 2319/70; C12N 5/16

(57) ABSTRACT

The present disclosure provides recombinantly manufactured ultra-long acting insulin-Fc fusion proteins for use in treating canine diabetes. The insulin-Fc fusion proteins comprise an insulin polypeptide linked via a peptide linker to an Fc-fragment of canine origin. Based on the results obtained, creating a treatment that is amenable to low cost manufacturing, exhibits sufficient in vivo bioactivity, displays extended duration of bioactivity, does not induce anti-drug antibodies, and substantially retains is potency over multiple administrations, requires a non-obvious combination of insulin polypeptide, peptide linkers, and species-specific Fc fragment, in addition to selective mutations on one or more of these components. Exemplary ultra-long acting insulin-Fc fusion proteins, polynucleotides encoding these insulin-Fc fusion proteins, and pharmaceutical formulations of exemplary insulin-Fc fusion proteins are provided, in addition to methods of use and preparation.

### PRIORITY AND RELATED APPLICATIONS

The present application is a continuation of U.S. patent application Ser. No. 17/523,133, filed Nov. 10, 2021, which is a continuation of U.S. patent application Ser. No. 17/135,333, filed Dec. 28, 2020, now U.S. Pat. No. 11,186,623, which claims the priority benefit of U.S. Patent Application Ser. No. 62/953,470, filed Dec. 24, 2019. The contents of the aforementioned patent applications are hereby incorporated herein by reference in their entireties.

### SEQUENCE LISTING

The following application contains a sequence listing submitted electronically as a Standard ST.26 compliant XML file, with a file name of “ABC-025-3USCN2.xml,” a creation date of Feb. 2, 2024, and a file size of 79,254 bytes in size, the contents of which are incorporated herein.

### TECHNICAL FIELD

The present technology relates to compositions of insulin-Fc fusion proteins and their use to treat diabetes in companion animals, e.g., dogs.

### BACKGROUND

The following description of the background of the present technology is provided simply as an aid in understanding the present technology and is not admitted to describe or constitute prior art to the present technology.

Diabetes is a chronic condition characterized by an insulin deficiency and/or ineffective use of insulin. Diabetics that have an absolute deficiency of insulin are categorized as having type 1 or insulin-dependent diabetes mellitus (IDDM). Type 1 diabetics are thought to have a genetic predisposition combined with immunologic destruction of the insulin-producing B-cells of the pancreas. In comparison, diabetics that can still produce some insulin but have a relative deficiency due to insulin resistance or other dysfunction, are classified as having type 2 or non-insulin-dependent diabetes mellitus (NIDDM). Type 2 diabetes is linked to genetic predisposition, obesity, and certain medications.

When a dog does not produce insulin or cannot use it normally, blood sugar levels elevate, resulting in hyperglycemia. Dogs generally exhibit an atypical glycemia phenotype with strong similarities to human type 1 diabetes. Dogs also occasionally exhibit atypical glycemia with strong similarities to type 2 diabetes in humans. Female dogs can also develop temporary insulin resistance while in heat or pregnant. In all cases, the dogs are treated with chronic insulin injection therapy. Left untreated, diabetes in dogs can lead to weight loss, loss of appetite, vomiting, dehydration, problems with motor function, coma, and even death.

Approximately 0.24% of dogs in the United States are affected by diabetes. Current diabetes therapies for dogs include the use of insulin, such as Vetsulin® for dogs (Intervet Inc., d.b.a. MERCK Animal Health, Summit, NJ) which is administered once or twice daily. The burden of frequent injections on owners often results in a lack of treatment regimen compliance and under-dosing, leading to poor long-term health outcomes. In fact, the cost of insulin therapy and the practicality of dosing their pets up to 14 times per week leads a significant percentage of owners to select euthanasia for their pets as an alternative to intensive management of diabetes. Therefore, there is a need for cost effective and less burdensome treatment options for this disease.

### SUMMARY OF THE PRESENT TECHNOLOGY

In an aspect, the present disclosure provides a fusion protein comprising an insulin polypeptide and an Fc fragment, wherein the insulin polypeptide and the Fc fragment are connected by a linker, such as a peptide linker, wherein the Fc fragment comprises one or more mutations to prevent natural glycosylation.

In one embodiment, the Fc fragment comprises the sequence:

(SEQ ID NO: 32)
CPKCPAPEMLGGPSVFIFPPKPKDTLLIARTPEVTCVVVDLDPEDPEVQI
SWFVDGKQMQTAKTQPREEQFKGTYRVVSVLPIGHQDWLKGKQFTCKVNN
KALPSPIERTISKARGQAHQPSVYVLPPSREELSKNTVSLTCLIKDFFPP
DIDVEWQSNGQQEPESKYRTTPPQLDEDGSYFLYSKLSVDKSRWQRGDTF
ICAVMHEALHNHYTQESLSHSPG.

In some embodiments, the insulin polypeptide comprises a C-peptide of the sequence: GGGPRR (SEQ ID NO: 8).

In an embodiment, the present disclosure provides for a fusion protein comprising an insulin polypeptide and an Fc fragment, wherein the insulin polypeptide and the Fc fragment are connected by a linker (e.g., peptide linker), and wherein the fusion protein comprises the sequence:

(SEQ ID NO: 51)
FVNQHLCGSDLVEALALVCGERGFFYTDPTGGGPRRGIVEQCCHSICSLY
QLENYCNGGGGAGGGGCPKCPAPEMLGGPSVFIFPPKPKDTLLIARTPEV
TCVVVDLDPEDPEVQISWFVDGKQMQTAKTQPREEQFKGTYRVVSVLPIG
HQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLY
SKLSVDKSRWQRGDTFICAVMHEALHNHYTQESLSHSPG.

In embodiments, the fusion protein comprises a dimer, wherein the dimer comprises two identical monomers bound together via disulfide bonds, e.g., the fusion protein is a homodimer. In some embodiments, the percentage homodimer of insulin-Fc fusion protein is greater than 90%.

In embodiments, the fusion proteins described herein are made using HEK293 cells, and the resulting homodimer titer after purification using Protein A beads or a Protein A column is greater than 40 mg/L. In embodiments, the insulin receptor IC50 for the fusion proteins described herein is less than or equal to 5000 nM. In embodiments, the serum half-life of the fusion proteins described herein in the blood or serum of a target animal upon administration is longer than about 3 days. In embodiments, for the fusion proteins described herein, the time during which there is a statistically significant decrease in blood glucose level in a subject relative to a pre-dose level is longer than one of 2 hours, 6 hours, 9 hours, 12 hours, 18 hours, 1 day, 1.5 days, 2 days, 2.5 days, 3 days, 4 days, 5 days, 6 days, 7 days, or longer.

In aspects, for the fusion proteins described herein, the NAOC after the first subcutaneous injection in a target animal is greater than 150% FBGL·days·kg/mg. In embodiments, for the fusion proteins described herein, the ratio of the NAOC after the third weekly subcutaneous injection of the fusion proteins in the target animal to the NAOC after the first subcutaneous injection of the fusion protein in the target animal is greater than 0.50.

In aspects, fusion proteins as described herein are formulated as a pharmaceutical composition. In embodiments, in the pharmaceutical composition the fusion protein is present at a concentration of about 3 mg/mL or greater. In embodiments, the composition is suitable for subcutaneous administration.

In aspects, a method is described for lowering the blood glucose level of a dog, the method comprising administering a physiologically effective amount of a fusion protein as described herein or a pharmaceutical composition thereof to the dog. In embodiments, the dog is diagnosed with diabetes. In some embodiments, the fusion protein is administered subcutaneously. In some embodiments, the fusion protein is administered daily, twice weekly, or once weekly to the dog. In examples, the fusion protein is administered once weekly to the dog at a dose between 0.025 and 0.5 mg/kg/week. In aspects, a cell engineered to express a fusion protein as described here in described. In examples, the cell is transfected with a nucleic acid encoding the fusion protein. In examples, the cell is a HEK293 cell or a CHO cell.

In an aspect, a cDNA encoding a fusion protein as described herein is described. In embodiments, the cDNA comprises the nucleic acid sequence encoding the insulin-Fc fusion protein of SEQ ID NO: 51):) where the leader is in bold: sequence

(SEQ ID NO: 52)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggatgccccaagtgccccgctcccgagatgctgggcggacccagcgtg
ttcatcttccctcccaagcccaaggacacactgctgatcgccaggacccc
ggaggtgacctgcgtggtggtggacctggatcccgaagaccccgaggtgc
agatcagctggttcgtggatggaaagcagatgcagaccgccaagacccaa
ccccgggaagagcagttcaaaggcacctacagggtggtgagtgtgttgcc
catcggccaccaggactggctgaaggggaagcaattcacatgcaaggtta
ataacaaggccctgcccagccccatcgagaggaccatcagcaaggccagg
ggccaggcccaccagccatctgtgtacgtgctgcccccatctagggagga
actgagcaagaacacagtcagccttacttgcctgatcaaggacttcttcc
caccggacatagacgtggagtggcagagtaacggccagcaggagcccgag
agcaagtataggaccacaccgccccaactggacgaggacggaagctactt
cctctacagcaaattgagcgttgacaaaagcaggtggcagcgaggcgaca
ccttcatctgcgccgtgatgcacgaggctttgcataaccactacacccag
gagagcctgtcccacagccccggatag.

### BRIEF DESCRIPTION OF THE DRAWINGS

FIG. 1 shows a schematic representation of an exemplary insulin-Fc fusion protein homodimer.

FIG. 2 shows average % fasting blood glucose levels from Day 0 to Day 3 for N=3 dogs dosed intravenously on Day 0 at 0.2 mg/kg with the homodimer of SEQ ID NO: 33.

FIG. 3 illustrates a side-by-side sequence comparison of SEQ ID NO: 33, SEQ ID NO: 35, SEQ ID NO: 37 and SEQ ID NO: 39. “*” represents complete homology across all sequences at a given sequence position, while “:”, “.” or spaces refer to conservative, moderate, or very different amino acid mutations across the sequences at a given sequence position respectively.

FIG. 4 illustrates a side-by-side sequence comparison of SEQ ID NO: 41, SEQ ID NO: 47, SEQ ID NO: 49 and SEQ ID NO: 51. “*” represents complete homology across all sequences at a given sequence position, while “:”, “.” or spaces refer to conservative, moderate, or very different amino acid mutations across the sequences at a given sequence position respectively.

FIG. 5 shows average % fasting blood glucose levels from Day 0 to Day 7 for N=3 dogs dosed intravenously on Day 0 at 0.2 mg/kg with the homodimer of SEQ ID NO: 41.

FIG. 6 shows average % fasting blood glucose levels from Day 0 to Day 7 for N=6 dogs dosed subcutaneously on Day 0 at 0.33 mg/kg with the homodimer of SEQ ID NO: 41.

FIG. 7 shows the average anti-drug antibody titer (μg/mL) for N=3 dogs dosed subcutaneously on Day 0 (0.30 mg/kg), Day 28 (0.33 mg/kg), Day 35 (0.33 mg/kg), Day 42 (0.50 mg/kg), Day 49 (1.00 mg/kg) and Day 56 (1.00 mg/kg) with the homodimer of SEQ ID NO: 41.

FIG. 8 shows % fasting blood glucose levels from Day 0 to Day 35 for N=1 dog dosed subcutaneously on Day 0, Day 14 and Day 21 with the homodimer of SEQ ID NO: 41.

FIG. 9 shows % fasting blood glucose levels from Day 0 to Day 40 for N=1 dog dosed subcutaneously on Day 0, Day 7, Day 14 and Day 21 with the homodimer of SEQ ID NO: 47.

FIG. 10 shows % fasting blood glucose levels from Day 0 to Day 35 for N=1 dog dosed subcutaneously on Day 0, Day 7, Day 14 and Day 21 with the homodimer of SEQ ID NO: 49.

FIG. 11 shows % fasting blood glucose levels from Day 0 to Day 40 for N=1 dog dosed subcutaneously on Day 0, Day 7, Day 14 and Day 21 with the homodimer of SEQ ID NO: 51.

FIG. 12 illustrates the “full aa sequence” of a fusion protein (SEQ ID NO: 51) and its corresponding nucleic acid sequence (SEQ ID NO: 52).

### DETAILED DESCRIPTION

An insulin treatment that requires less frequent dosing (e.g., once-weekly injections) would be less burdensome on the owners, leading to better compliance, fewer instances of euthanasia, and better outcomes for the pets. As disclosed herein, proposed ultra-long acting insulin treatments for veterinary clinical use comprise an insulin-Fc fusion protein making use of an Fc fragment to prolong their action in vivo. An insulin-Fc fusion protein suitable for an ultra-long acting treatment for diabetes should meet various design goals. An insulin-Fc fusion protein configuration suitable for an ultra-long acting treatment for diabetes in dogs should be manufacturable in mammalian cells, for example human embryonic kidney (HEK, e.g. HEK293) cells, with an acceptable titer of the desired homodimer product (e.g., greater than 40 mg/L homodimer titer from transiently transfected HEK cells, greater than 75 mg/L from transiently transfected from HEK cells, greater than 100 mg/L from transiently transfected HEK cells, etc.). Only candidates with a homodimer titer of greater than 40 mg/L from transiently transfected HEK cells are considered useful in the present invention, because experience has demonstrated that homodimer titers less than this level will not likely result in commercial production homodimer titers in Chinese hamster ovary (CHO) cells that meet the stringently low manufacturing cost requirements for veterinary products.

In addition, the molecule must bind the insulin receptor with an appreciable affinity (e.g., IC50 less than 5000 nM, IC50 less than 4000 nM, IC50 less than 3000 nM, IC50 less than 2500 nM, etc.) as measured in the 4° C. IM-9 insulin receptor binding assay. Based on experience, only molecules exhibiting insulin receptor activity IC50 values less than 5000 nM are deemed likely to exhibit the requisite bioactivity in the target species. The molecule must also demonstrate sustained bioactivity in vivo (e.g., demonstrate glucose lowering activity greater than about 2 hours, 6 hours, 9 hours, 12 hours, 18 hours, 1 day, 1.5 days, 2 days, 2.5 days, 3 days, 4 days, 5 days, 6 days, 7 days, or longer) to justify less frequent dosing. The molecule must also demonstrate prolonged system residence time in the target animal (e.g., the serum half-life must be greater than 3 days, or longer). The bioactive potency and duration of the bioactivity may be quantitatively represented by calculating the area over the percent fasting blood glucose (% FBGL) curve normalized to a given dose in mg/kg (NAOC) with units of % FBGL·days·kg/mg as described in Example 15. The NAOC increases with a greater drop in % FBGL, which is the case where the molecule demonstrates increased bioactivity, and when the % FBGL takes longer to return to 100%, which is the case where the insulin-Fc fusion protein demonstrates increased duration of action. To be useful as described herein, a molecule must demonstrate a sufficiently high NAOC value (e.g. preferably NAOC greater than 150% FBGL.days.kg/mg, more preferably NAOC greater than 200% FBGL·days·kg/mg, and even more preferably NAOC greater than 250% FBGL·days·kg/mg). Based on experience, at NAOC values greater than 150% FBGL·days·kg/mg, the dose requirements in the target species will be sufficiently low so as to reach an acceptable treatment cost.

Proposed ultra-long acting insulin treatments for veterinary clinical use in dogs comprise an insulin-Fc fusion protein making use of an Fc fragment to prolong the action in vivo.

Provided herein, therefore, are manufacturable, high purity, long-acting, bioactive, insulin-Fc fusion protein configurations comprising an insulin polypeptide, an Fc fragment, and a linker between the insulin polypeptide and the Fc fragment, and which exhibit acceptably high homodimer titers (e.g., homodimer titers greater than 40 mg/L), acceptable IR binding (e.g., insulin receptor activity IC50 less than 5000 nM, IC50 less than 4000 nM, IC50 less than 3000 nM, IC50 less than 2500 nM, etc.) and/or acceptable bioactivity in vivo (e.g., NAOC values greater than 150% FBGL·days·kg/mg). These exemplary insulin-Fc fusion protein configurations are expected to exhibit sufficiently high insulin receptor binding and a long enough half-life to justify repeated once-a-weck dosing, rendering them suitable for the treatment of diabetes in dogs.

### Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

As used herein, an amount of a molecule, compound, conjugate, or substance effective to treat a disorder (e.g., a disorder described herein), “therapeutically effective amount,” or “effective amount” refers to an amount of the molecule, compound, conjugate, or substance which is effective, upon single or multiple dose administration(s) to a subject, in treating a subject, or in curing, alleviating, relieving or improving a subject with a disorder (e.g., a disorder described herein) beyond that expected in the absence of such treatment.

As used herein, the term “analog” refers to a compound or conjugate (e.g., a compound or conjugate as described herein, e.g., insulin) having a chemical structure similar to that of another compound or conjugate but differing from it in at least one aspect.

As used herein, the term “antibody” or “antibody molecule” refers to an immunoglobulin molecule (Ig), immunologically active portions of an immunoglobulin (Ig) molecule, i.e., a molecule that contains an antigen binding site that specifically binds, e.g., immunoreacts with, an antigen. As used herein, the term “antibody domain” refers to a variable or constant region of an immunoglobulin. It is documented in the art that antibodies comprise several classes, for example IgA, IgM, or IgG in the case of mammals (e.g., humans and dogs). Classes of immunoglobulins can be further classified into different isotypes, such as IgGA, IgGB, IgGC, and IgGD for canines. Those skilled in the art will recognize that immunoglobulin isotypes of a given immunoglobulin class will comprise different amino acid sequences, structures, and functional properties from one another (e.g., different binding affinities to Fc(gamma) receptors). “Specifically binds” or “immunoreacts with” means that the antibody reacts with one or more antigenic determinants of the desired antigen and has a lower affinity for other polypeptides, e.g., docs not react with other polypeptides.

As used herein, the term “area-under-the-curve” or “AUC” refers to the integrated area under the % FBGL vs. time curve for a subject after a given dose of an insulin-Fc fusion protein configuration is administered. As used herein, the term “area-over-the curve” or “AOC” is used as a measure of the biological potency of an insulin-Fc fusion protein configuration such that the AOC equals the difference between the total possible area under the % FBGL vs. time curve and the AUC value. As used herein, the “normalized arca-over-the curve,” “normalized AOC,” or “NAOC” is the AOC value divided by the actual dose of insulin-Fc fusion protein administered. As used herein, the term “normalized AOC ratio” or “NAOCR” is the ratio of the NAOC resulting from a particular administration of an insulin-Fc fusion protein to the NAOC resulting from the first administration of an insulin-Fc fusion protein in a series of administrations. The NAOCR thus provides a measure of the change in biological activity of an insulin-Fc fusion protein after repeated administrations.

As used herein, the term “bioactivity,” “activity,” “biological activity,” “potency,” “bioactive potency,” or “biological potency” refers to the extent to which an insulin-Fc fusion protein activates the insulin receptor and/or exerts a reduction in blood glucose levels in a target subject. As used herein, “in vitro activity” or “insulin receptor activity” refers to the affinity with which an insulin-Fc fusion protein binds to the insulin receptor and is typically measured by the concentration at which an insulin-Fc fusion protein displaces half of an insulin reference standard from the insulin receptor in a competitive binding assay (i.e., IC50). As used herein, “in vivo activity” refers to the extent and duration of reduction in a target subject's fasting blood glucose level after administration of an insulin-Fc fusion protein.

As used herein, the term “biosynthesis,” “recombinant synthesis,” or “recombinantly made” refers to the process by which an insulin-Fc fusion protein is expressed within a host cell by transfecting the cell with a nucleic acid molecule (e.g., vector) encoding the insulin-Fc fusion protein (e.g., where the entire insulin-Fc fusion protein is encoded by a single nucleic acid molecule). Exemplary host cells include mammalian cells, e.g., HEK293 cells or CHO cells. The cells can be cultured using standard methods in the art and the expressed insulin-Fc fusion protein may be harvested and purified from the cell culture using standard methods in the art.

As used herein, the term “cell surface receptor” refers to a molecule such as a protein, generally found on the external surface of the membrane of a cell and which interacts with soluble molecules, e.g., molecules that circulate in the blood supply. In some embodiments, a cell surface receptor may include a hormone receptor (e.g., an insulin hormone receptor or insulin receptor (IR)) or an Fc receptor which binds to an Fc fragment or the Fc region of an antibody (e.g. an Fc(gamma) receptor, for example Fc(gamma) receptor I, or an Fc neonatal receptor, for example FcRn). As used herein, “in vitro activity” or “Fc(gamma) receptor activity” or “Fc(gamma) receptor binding” or “FcRn receptor activity” or “FcRn binding” refers to the affinity with which an insulin-Fc fusion protein binds to the Fc receptor (e.g. Fc(gamma) receptor or FcRn receptor) and is typically measured by the concentration of an insulin-Fc fusion protein that causes the insulin-Fc fusion protein to reach half of its maximum binding (i.e., EC50 valuc) as measured on an assay (e.g., an enzyme-linked immunosorbent assay (ELISA) assay) using OD 450 nm values as measured on a microplate reader. Alternatively, the affinity with which an insulin-Fc fusion protein binds to the Fc receptor (e.g. Fc(gamma) receptor or FcRn receptor) is measured by the OD 450 nm value obtained on a microplate reader in an enzyme-linked immunosorbent assay (ELISA) assay at a given concentration of the insulin-Fc fusion protein.

As used herein, the term “fasting blood glucose level” or “FBGL” refers to the average blood glucose level in a target subject at the end of a period during which no food is administered and just prior to the time at which an insulin-Fc fusion protein is administered. As used herein, the term “percent fasting blood glucose level,” “% fasting blood glucose level,” or “% FBGL” refers to the ratio of a given blood glucose level to the fasting blood glucose level multiplied by 100.

As used herein, the term “immunogenic” or “immunogenicity” refers to the capacity for a given molecule (e.g., an insulin-Fc fusion protein of the present invention) to provoke the immune system of a target subject.

As used herein, the term “insulin reference standard” is any one of: (i) a naturally occurring insulin from a mammal (e.g., a human or dog); (ii) an insulin polypeptide that does not comprise an Fc fragment; or (iii) a standard of care insulin (e.g., a commercially available insulin).

As used herein, the term “monomer” refers to a protein or a fusion protein comprising a single polypeptide. In embodiments, the “monomer” is a protein or a fusion protein, e.g., a single polypeptide, comprising an insulin polypeptide and an Fc fragment polypeptide, wherein the insulin polypeptide and Fc fragment polypeptide are joined by a peptide linker to form the single polypeptide. In embodiments, the monomer is encoded by a single nucleic acid molecule.

As used herein, “N-terminus” refers to the start of a protein or polypeptide that is initiated by an amino acid containing a free amine group that is the alpha-amino group of the amino acid (e.g. the free amino that is covalently linked to one carbon atom that is located adjacent to a second carbon atom, wherein the second carbon atom is part of the carbonyl group of the amino acid). As used herein, “C-terminus” refers to the end of a protein or polypeptide that is terminated by an amino acid containing a carboxylic acid group, wherein the carbon atom of the carboxylic acid group is located adjacent to the alpha-amino group of the amino acid.

As used herein, “pharmacodynamics” or “PD” generally refers to the biological effects of an insulin-Fc fusion protein in a subject. Specifically, herein the PD refers to the measure of the reduction in fasting blood glucose level over time in a subject after the administration of an insulin-Fc fusion protein.

As used herein, “pharmacokinetics” or “PK” generally refers to the characteristic interactions of an insulin-Fc fusion protein and the body of the subject in terms of its absorption, distribution, metabolism, and excretion. Specifically, herein the PK refers to the concentration of an insulin-Fc fusion protein in the blood or serum of a subject at a given time after the administration of the insulin-Fc fusion protein. As used herein, “half-life” refers to the time taken for the concentration of insulin-Fc fusion protein in the blood or serum of a subject to reach half of its original value as calculated from a first order exponential decay model for drug elimination. Insulin-Fc fusion proteins with greater “half-life” values demonstrate greater duration of action in the target subject.

The terms “sequence identity” “sequence homology” “homology” or “identical” in amino acid or nucleotide sequences as used herein describes that the same nucleotides or amino acid residues are found within the variant and reference sequences when a specified, contiguous segment of the nucleotide sequence or amino acid sequence of the variant is aligned and compared to the nucleotide sequence or amino acid sequence of the reference sequence. Methods for sequence alignment and for determining identity between sequences are known in the art, including the use of Clustal Omega, which organizes, aligns, and compares sequences for similarity, wherein the software highlights each sequence position and compares across all sequences at that position and assigns one of the following scores: an “*” (asterisk) for sequence positions which have a single, fully conserved residue, a “:” (colon) indicates conservation between groups of strongly similar properties with scoring greater than 0.5 in the Gonnet PAM 250 matrix, and a “.” (period) indicates conservation between groups of weakly similar properties with scoring less than or equal to 0.5 in the Gonnet PAM 250 matrix, a “-” (dash) indicates a sequence gap, meaning that no local homology exists within a particular set of comparisons within a certain range of the sequences, and an empty space “ ” indicates little or no sequence homology for that particular position across the compared sequences. See, for example Ausubel et al., eds. (1995) Current Protocols in Molecular Biology, Chapter 19 (Greene Publishing and Wiley- Interscience, New York); and the ALIGN program (Dayhoff (1978) in Atlas of Polypeptide Sequence and Structure 5: Suppl. 3 (National Biomedical Research Foundation, Washington, D.C.). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. In some embodiments, the contiguous segment used for comparison to the reference nucleotide sequence or reference amino acid sequence will comprise at least 6, 10, 15, or 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are known in the art.

In embodiments, the determination of percent identity or “homology” between two sequences is accomplished using a mathematical algorithm. For example, the percent identity of an amino acid sequence is determined using the Smith-Waterman homology search algorithm using an affine 6 gap search with a gap open penalty of 12 and a gap extension penalty of 2, BLOSUM matrix 62. The Smith-Waterman homology search algorithm is described in Smith and Waterman (1981) Adv. Appl. Math 2:482-489, herein incorporated by reference. In embodiments, the percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

As used herein, the term “homology” is used to compare two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling) which works by finding similar sequences on the basis of the fact that 3D similarity reflects 2D similarity. Homologous structures do not imply sequence similarity as a necessary condition.

As used herein, the terms “subject” or “target subject” or “patient” or “patient subject” are intended to include canines. Exemplary canine subjects include dogs having a discase or a disorder, e.g., diabetes or another disease or disorder described herein, or normal subjects.

As used herein, the term “titer” or “yield” refers to the amount of a fusion protein product (e.g., an insulin-Fc fusion protein described herein) resulting from the biosynthesis (e.g., in a mammalian cell, e.g., in a HEK293 cell or CHO cell) per volume of the cell culture. The amount of product may be determined at any step of the production process (e.g., before or after purification), but the yield or titer is always stated per volume of the original cell culture. As used herein, the term “product yield” or “total protein yield” refers to the total amount of insulin-Fc fusion protein expressed by cells and purified via at least one affinity chromatography step (e.g. Protein A or Protein G) and includes monomers of insulin-Fc fusion protein, homodimers of insulin-Fc fusion protein, and higher-order molecular aggregates of homodimers of insulin-Fc fusion protein. As used herein, the term “percent homodimer” or “% homodimer” refers to the proportion of a fusion protein product (e.g., an insulin-Fc fusion protein described herein) that is the desired homodimer. As used herein, the term “homodimer titer” refers to the product of the % homodimer and the total protein yield after Protein A purification step reported per volume of the cell culture.

As used herein, the terms “treat” or “treating” a subject having a disease or a disorder refer to subjecting the subject to a regimen, for example the administration of a fusion protein such as a fusion protein described herein, such that at least one symptom of the disease or disorder is cured, healed, alleviated, relieved, altered, remedied, ameliorated, or improved. Treating includes administering an amount effective to alleviate, relieve, alter, remedy, ameliorate, improve or affect the disease or disorder, or the symptoms of the disease or disorder. The treatment may inhibit deterioration or worsening of a symptom of a disease or disorder.

### Insulin-Fc Fusion Protein Components and Structure

The present disclosure relates to a composition of a fusion protein (i.e., an insulin-Fc fusion protein) comprising an insulin polypeptide linked via a peptide linker to a species-specific Fc fragment, and its use to treat diabetes in dogs. As used herein, the terms “fusion protein” and “insulin-Fc fusion protein” refer to a protein comprising more than one part, for example from different sources (different proteins, polypeptides, cells, etc.), that are covalently linked through peptide bonds. The insulin-Fc fusion proteins are covalently linked by (i) connecting the genes that encode for each part into a single nucleic acid molecule and (ii) expressing in a host cell (e.g., HEK or CHO) the protein for which the nucleic acid molecule encodes as follows: (N-terminus)—insulin polypeptide—linker—Fc fragment—(C-terminus). The fully recombinant synthesis approach is preferred over methods in which the insulin polypeptide and Fc fragments are synthesized separately and then chemically conjugated. The chemical conjugation step and subsequent purification process increase the manufacturing complexity, reduce product yield, and increase cost.

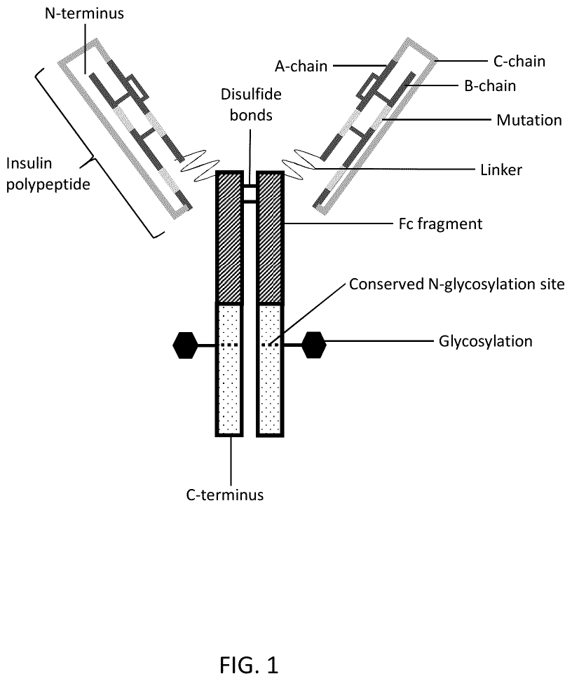

As used herein, the term “dimer” refers to a protein or a fusion protein comprising two polypeptides linked covalently. In embodiments, two identical polypeptides are linked covalently (e.g., via disulfide bonds) forming a “homodimer”. FIG. 1 diagrammatically represents an insulin-Fc fusion protein homodimer. The insulin polypeptide comprises a B-chain, a C-chain and an A-chain, and cach insulin polypeptide is joined via a peptide linker to an Fc fragment. Two identical insulin-Fc fusion protein monomers are joined together vis disulfide bonds to form an insulin-Fc fusion protein homodimer. The total number of disulfide bonds in actuality may be greater or less than the number shown in FIG. 1. The Fc fragment comprises a conserver asparagine (N)-glycosylation site, at which glycols may bond during recombinant manufacturing of the insulin-Fc fusion protein in mammalian cells. In embodiments, the homodimer is encoded by a single nucleic acid molecule, wherein the homodimer is made recombinantly inside a cell by first forming insulin-Fc fusion protein monomers and by then assembling two identical insulin-Fc fusion protein monomers into the homodimer upon further processing inside the cell.

As used herein, the terms “multimer,” “multimeric,” or “multimeric state” refer to non-covalent, associated forms of Fc fusion protein dimers that may be in equilibrium with Fc fusion protein dimers or may act as permanently aggregated versions of Fc fusion protein dimers (e.g., dimers of Fc fusion protein homodimers, trimers of Fc fusion protein homodimers, tetramers of Fc fusion protein homodimers, or higher order aggregates containing five or more Fc fusion protein homodimers). It may be expected that multimeric forms of Fc fusion proteins may have different physical, stability, or pharmacologic activities from that of the insulin-Fc fusion protein homodimers.

### Insulin Polypeptide

An insulin polypeptide may be, for example, an insulin or insulin analog produced by B-cells in the islets of Langerhans within the pancreas. Insulin functions by regulating the absorption of glucose from the blood. Upon a stimulus, such as increased protein and glucose levels, insulin is released from β-cells and binds to the insulin receptor (IR), initiating a signal cascade that affects many aspects of mammalian (e.g., human) metabolism. Disruption of this process is directly related to several diseases, notably diabetes, insulinoma, insulin resistance, metabolic syndromes, and polycystic ovary syndrome. Insulin analogs of the present disclosure may be related to the structure of insulin yet contain one or more modifications. In some embodiments, the insulin analog comprises at least one amino acid substitution, deletion, addition or chemical modification relative to insulin, which may impact a particular feature or characteristic of the insulin-Fc fusion protein configuration. For example, the modifications or alterations described herein may impact the structure, stability, pH sensitivity, bioactivity, or binding affinity of the insulin-Fc fusion protein configuration to a cell surface receptor (e.g. an insulin hormone receptor) relative to a reference standard.

The amino acid sequence of insulin is strongly conserved throughout evolution, particularly in vertebrates. For example, native canine and porcine insulins differ by only one amino acid from human insulin, native bovine insulin differs by only three amino acids from human insulin, and native feline insulin differs by just four amino acids from human insulin. As used herein, the terms “B-chain or B-chain analog”, “C-peptide” or “C-chain”, and “A-chain or A-chain analog” refer to the peptide segments of an insulin polypeptide as illustrated in FIG. 1. Native insulin is a 51 amino acid hormone containing two peptide chains (i.e., a B-chain and an A-chain) connected via disulfide bonds (e.g., disulfide bonds formed by one or more B-chain cysteine side chain thiols and one or more A-chain cysteine side chain thiols). The A-chain of native insulin is 21 amino acids in length and the B-chain of native insulin is 30 amino acids in length. In the native form of insulin, the A-chain contains one intrachain disulfide bond formed by two A-chain cysteine side chain thiols. For reference purposes, the sequences for the human insulin B-chain of SEQ ID NO: 1 and the human insulin A-chain of SEQ ID NO: 2 are shown below:

FVNQHLCGSHLVEALYLVCGERGFFYTPKT (SEQ ID NO: 1)

GIVEQCCTSICSLYQLENYCN (SEQ ID NO: 2).

As used herein, the term “insulin” or “insulin polypeptide” encompasses mature insulin, preproinsulin, proinsulin, and naturally occurring insulin, or analogs thereof. In embodiments, an insulin polypeptide can be a full-length insulin polypeptide or a fragment thereof. In embodiments, an insulin polypeptide can comprise one or more fragments from mature insulin, preproinsulin, proinsulin, or naturally occurring insulin.

Insulin is normally constructed as a N-terminus—B-chain:C-chain: A-chain—C-terminus polypeptide, wherein the C-chain is cleaved in order to make it bioactive. For reference purposes, the sequence of the entire human insulin molecule including the C-chain (i.e., human proinsulin) is shown below with the C-chain in bold:

FVNQHLCGSHLVEALYLVCGERGFFYTPKTRREAEDLQVGQVELGGGPGAGSLQPLALEGS LOKRGIVEQCCTSICSLYQLENYCN (SEQ ID NO: 3).

The transformation of the single-chain insulin polypeptide into a bioactive two-chain polypeptide is normally accomplished within the β-cells of the islets of Langerhans prior to glucose-stimulated insulin secretion by two endoproteases, Type I endoproteases, PC1 and PC3, that disrupt the C peptide-B chain connection and PC2, and a Type II endoprotease, that cleaves the C peptide-A chain bond at exactly the right sites. However, cell systems used for the biosynthesis of therapeutic molecules such as insulin (e.g. bacteria, yeast, and mammalian (e.g. HEK and CHO) cell systems) do not possess this pathway, and therefore the transformation must take place after expression and harvesting of the single chain polypeptide using chemical or enzymatic methods. All the known techniques for cleaving the C-chain after expression and harvesting rely on first modifying the C-chain such that it terminates in a lysine just before the N-terminus of the A-chain. Then, using an enzyme selected from the trypsin or Lys-C families, which clips peptide bonds specifically at the C-termini of lysine residues, the single chain-insulin polypeptide is cleaved at the C-terminal lysine of the C-chain and at the C-terminal lysine at the 29^{th} position from the N-terminus of the B-chain. In some cases, the resulting bioactive two-chain insulin is used without reattaching the clipped amino acid at the 30^{th} position from the N-terminus of the B-chain, and in some cases the clipped amino acid at the 30^{th} position from the N-terminus of the B-chain is added back to the molecule using an additional enzymatic method. Such a process works well with insulin because it contains only one lysine in its entire two chain polypeptide form. However, this process cannot be used on the insulin-Fc fusion proteins contained herein, because all known Fc fragments contain multiple lysine residues. The enzymatic cleavage process would, therefore, digest the Fc fragment into non-functional parts, thereby eliminating the ability of the Fc fragment to prolong the action of the insulin polypeptide in vivo. Therefore, an insulin-Fc fusion protein of the present invention must comprise an insulin polypeptide that does not require C-chain cleavage and is therefore bioactive in its single chain form.

A number of bioactive single chain insulin polypeptides have been described in the art. In all cases, the single chain insulin polypeptides contain C-chains of specific length and composition as well as A-chains and B-chains mutated at specific amino acid sites in order to achieve electrostatic balance, prevent aggregation, and enhance insulin receptor (IR) binding and/or downstream signaling to achieve bioactivity at levels comparable to that of the native two-chain insulin. Herein, the location of mutations on peptide segments are notated using the name of the segment (e.g., B-chain, C-chain, A-chain) and the number of the amino acid counting from the N-terminus of the segment. For example, the notation “B10” refers to the 10^{th} amino acid from the N-terminus of the amino acid sequence of the B-chain. The notation “A8” refers to the 8^{th} amino acid from the N-terminus of the A-chain. Furthermore, if an amino acid is mutated from its native form to a new amino acid at a particular location, the location is appended with the one letter amino acid code for the new amino acid. For example, B10D refers to an aspartic acid mutation at the 10^{th} amino acid from the N-terminus of the amino acid sequence of the B-chain and A8H refers to a histidine mutation at the 8^{th} amino acid from the N-terminus of the amino acid sequence of the A-chain.

In examples, a single chain insulin analog comprises a C-chain 4-9 amino acids in length comprising a specific set of alanine substitutions and/or deletions derived from a C-chain of the sequence GGGPRR (SEQ ID NO: 8).

In some examples, the C-chain comprises the following sequence:

X₁X₂X₃X₄X₅X₆X₇RR (SEQ ID NO: 9)
wherein X1 is E or absent, X2 is E or absent, X3 is E, G or absent, X₄ is G, E, A, or absent, X₅ is G, E, A, F, or D, X₆ is G, E, or A, and X₇ is P or A.

In some examples, the C-chain comprises the following sequence:

EX₁X₂X₃X₄A (SEQ ID NO: 10)
wherein X₁ is E or A, X₂ is G or A, X₃ is A or P, and X₄ is A or R.

In some examples, the C-chain comprises the following sequence:

X₁X₂GPR (SEQ ID NO: 11)
wherein X₁ is E or absent, and X₂ is E or A.

In some examples, the C-chain comprises the following sequence:

X₁EGX₂X₃X₄ (SEQ ID NO: 12)
wherein X₁ is E or absent, X₂ A or P, X₃ is A or R, and X₄ is A or R.

SEQ ID NO: 15 was developed by The Department of Biochemistry, Case Western Reserve University School of Medicine and the Department of Medicine, University of Chicago (see Hua, Q.-x, Nakagawa, S. H., Jia, W., Huang, K., Phillips, N. B., Hu, S.-q., Weiss, M. A., (2008) J. Biol. Chem Vol. 283, No. 21 pp 14703-14716). In this example, at position 8 of the A-chain (i.e., A8), histidine is substituted for threonine; at position 10 of the B-chain (i.e., B10), aspartic acid is substituted for histidine; at position 28 of the B-chain (i.e., B28), aspartic acid is substituted for proline; and at position 29 of the B-chain (i.e., B29), proline is substituted for lysine. The C-chain comprises SEQ ID NO: 8. SEQ ID NO: 15 is listed below with each of the non-native amino acids in bold:

FVNQHLCGSDLVEALYLVCGERGFFYTDPTGGGPRRGIVEQCCHSICSLYQLENYCN (SEQ ID NO: 15).

In embodiments, alanine may be substituted for tyrosine at position 16 from the N-terminus of the B-chain (i.e., B16) in SEQ ID NO: 15 to produce SEQ ID NO: 16, as an alanine substitution in this position is known to be less capable of activating insulin-specific T cells (Alleva, D. G., Gaur, A., Jin, L., Wegmann, D., Gottlieb, P. A., Pahuja, A., Johnson, E. B., Motheral, T., Putnam, A., Crowe, P. D., Ling, N., Bochme, S. A., Conlon, P. J., (2002) Diabetes Vol. 51, No. 7 pp 2126-2134). SEQ ID NO: 16 is listed below with each of the non-native amino acids in bold:

FVNQHLCGSDLVEALALVCGERGFFYTDPTGGGPRRGIVEQCCHSICSLYQLENYCN (SEQ ID NO: 16).

In some embodiments, it was unexpectedly discovered that specific amino acids in SEQ ID NO: 15 and SEQ ID NO: 16 led to the development of neutralizing anti-drug antibodies after repeated subcutaneous injections in the target animal (e.g., dog or cat) as is illustrated in Example 26. The anti-drug antibodies led to an unacceptable reduction in the NAOC after multiple injections (e.g., a NAOCR value after the third injection of less than 0.5), rendering the associated insulin-Fc fusion proteins non-viable. Specifically, it was discovered in the steps leading up to the invention of this disclosure that the A8 mutation to histidine and the B10 mutation to aspartic acid accounted for the vast majority of the anti-drug antibody specificity and thus represented immunogenic “hot spots” (e.g. immunogenic epitopes) on the insulin-polypeptide.

In addition to the B-Chain of SEQ ID NO: 15 (FVNQHLCGSDLVEALYLVCGERGFFYTDPT—SEQ ID NO: 5) and the B-chain of SEQ ID NO: 16 (FVNQHLCGSDLVEALALVCGERGFFYTDPT—SEQ ID NO: 4), other examples of B-chains that may be substituted for SEQ ID NO: 4 and SEQ ID NO: 5 in the insulin polypeptides of SEQ ID NO: 15 and SEQ ID NO: 16 include FVNQHLCGSHLVEALYLVCGERGFFYTX₁X₂PT, wherein X₁X₂ is PR, PE, PA, DP, EP, AP, DK, EK, ER, AK, or AR (SEQ ID NO: 6) and FVNQHLCGSHLVEALYLVCGERGFFYTKPT (SEQ ID NO: 7).

In some embodiments, in addition to the A-Chain of SEQ ID NO: 15 and SEQ ID NO: 16 (GIVEQCCHSICSLYQLENYCN—SEQ ID NO: 14), other examples of A-chains that may be substituted for SEQ ID NO: 14 in the insulin polypeptides of SEQ ID NO: 15 and SEQ ID NO: 16 include GIVEQCCX1SICSLX2QLENYCN wherein X1 is T, H, S, E or A and wherein X2 is any amino acid other than P (SEQ ID NO: 13).

Table 1 illustrates the A-Chain analogs, C-Chain Analogs, and B-Chain analogs which may be selected from, in any combination in forming an insulin polypeptide of the present invention.

TABLE 1
A-Chain, C-Chain and B-Chains that may be selected
in any combination to form an insulin polypeptide

| SEQ | | SEQ | | SEQ | |
|---|---|---|---|---|---|
| ID NO: | B-Chain | ID NO: | C-Chain | ID NO: | A-Chain |
| SEQ ID | FVNQHLCGSDLVEALA | SEQ ID | X1X2X3X4X5X6X7RR | SEQ ID | GIVEQCCX₁SICSLX₂QLE |
| NO: 4 | LVCGERGFFYTDPT | NO: 9 | wherein X1 is E or absent, | NO: 13 | NYCN wherein X₁ is |
| | | | X2 is E or absent, X3 is E, | | T, H, S, E or A |
| | | | G or absent, X4 is G, E, A, | | and wherein X₂ is |
| | | | or absent, X5 is G, E, A, F, | | any amino acid |
| | | | or D, X6 is G, E, or A, and | | other than P |
| | | | X7 is P or A. | | |
| SEQ ID | FVNQHLCGSDLVEALY | SEQ ID | EX₁X₂X₃X₄A wherein X₁ | SEQ ID | GIVEQCCHSICSLYQLE |
| NO: 5 | LVCGERGFFYTDPT | NO: 10 | is E or A, X₂ is G or A, X₃ | NO: 14 | NYCN |
| | | | is A or P, and X₄ is A or R. | | |
| SEQ ID | FVNQHLCGSHLVEALY | SEQ ID | X₁X₂GPR wherein X₁ is E | | |
| NO: 6 | LVCGERGFFYTX₁X₂PT, | NO: 11 | or absent, and X₂ is E or A. | | |
| | wherein X₁X² is | | | | |
| | PR, PE, PA, DP, | | | | |
| | EP, AP, DK, EK, | | | | |
| | ER, AK, or AR | | | | |
| SEQ ID | FVNQHLCGSHLVEALY | SEQ ID | X₁EGX₂X₃X₄ wherein X₁ | | |
| NO: 7 | LVCGERGFFYTKPT | NO: 12 | is E or absent, X₂ A or P, | | |
| | | | X₃ is A or R, and X₄ is | | |
| | | | A or R. | | |

In some embodiments, the insulin polypeptide comprises the following sequence: FVNQHLCGSHLVEALYLVCGERGFFYTDPTEEGPRRGIVEQCCHSICSLEQLENYCN (SEQ ID NO: 17). In some embodiments, the insulin polypeptide comprises the following sequence: FVNQHLCGSHLVEALYLVCGERGFFYTDPTEEGAPRRGIVEQCCHSICSLEQLENYCN (SEQ ID NO: 18). In some embodiments, the insulin polypeptide comprises the following sequence: FVNQHLCGSHLVEALYLVCGERGFFYTDPTEEGAAPRRGIVEQCCHSICSLEQLENYCN (SEQ ID NO: 19). In some embodiments, the insulin polypeptide comprises the following sequence: FVNQHLCGSHLVEALYLVCGERGFFYTDPTEEGAAAPRRGIVEQCCHSICSLEQLENYCN (SEQ ID NO: 20).

### Linker

In some examples, the C-terminus of the insulin polypeptide is connected directly to the N-terminus of the Fc fragment (e.g. no linker or linker absent). In other examples, the successful construction of a recombinantly made insulin-Fc fusion protein requires a linker connecting the insulin polypeptide to the Fc fragment. In embodiments, an insulin-Fc fusion protein configurations described herein comprise a peptide linker between the insulin polypeptide and the Fc fragment comprising amino acids (e.g., natural or unnatural amino acids). In embodiments, the peptide linker can be encoded by a nucleic acid molecule, for example such that a single nucleic acid molecule can encode the various peptides within an insulin polypeptide as well as the peptide linker and the Fc fragment. The choice of peptide linker (for example, the length, composition, hydrophobicity, and secondary structure) could impact the manufacturability (i.e., the homodimer titer), the chemical and enzymatic stability, the bioactivity (i.e., the NAOC valuc), and the immunogenicity of the insulin-Fc fusion protein (Chen, X., Zaro, J., Shen, W. C., Adv Drug Deliv Rev. 2013 October 15; 65(10): 1357-1369). In embodiments, the peptide linker comprises the sequence GGGGAGGGG (SEQ ID NO: 25). In embodiments, the peptide linker comprises the sequence GGGGSGGGG (SEQ ID NO: 26). In embodiments, the peptide linker comprises the sequence GGGGGQGGGGQGGGGQGGGGG (SEQ ID NO: 27).

### Fc Fragment

In embodiments, the Fc fragment comprises the Fc region (e.g., hinge region, CH2 domain, and CH3 domain) of a mammalian IgG, for example a canine IgGA Fc fragment (SEQ ID NO: 28), a canine IgGB Fc fragment (SEQ ID NO: 29), a canine IgGC Fc fragment (SEQ ID NO: 30), or a canine IgGD Fc fragment (SEQ ID NO: 31). In embodiments, the C-terminal lysine that is often found in native canine or IgG isotype Fc fragment amino acid sequences (i.e., the lysine that represents the last amino acid of the Fc fragment sequence) is omitted to prevent the accidental production of unwanted amino acid sequence variants during manufacturing (e.g., Fc fragments containing the C-terminal lysine becoming mixed with Fc fragments where the C-terminal lysine is omitted, which can occur during production of the desired protein within cells (Dick, L W., (2008) Biotechnol Bioeng. Aug 15;100(6) pp1132-43). Therefore, in embodiments, the canine Fc fragment sequences lacking a C-terminal lysine are:

(SEQ ID NO: 28)
Canine IgGA
RCTDTPPCPVPEPLGGPSVLIFPPKPKDILRITRTPEVTCVVLDLGREDP
EVQISWFVDGKEVHTAKTQSREQQFNGTYRVVSVLPIEHQDWLTGKEFKC
RVNHIDLPSPIERTISKARGRAHKPSVYVLPPSPKELSSSDTVSITCLIK
DFYPPDIDVEWQSNGQQEPERKHRMTPPQLDEDGSYFLYSKLSVDKSRWQ
QGDPFTCAVMHETLQNHYTDLSLSHSPG
Canine IgGB
(SEQ ID NO: 29)
DCPKCPAPEMLGGPSVFIFPPKPKDTLLIARTPEVTCVVVDLDPEDPEVQ
ISWFVDGKQMQTAKTQPREEQFNGTYRVVSVLPIGHQDWLKGKQFTCKVN
NKALPSPIERTISKARGQAHQPSVYVLPPSREELSKNTVSLTCLIKDFFP
PDIDVEWQSNGQQEPESKYRTTPPQLDEDGSYFLYSKLSVDKSRWQRGDT
FICAVMHEALHNHYTQESLSHSPG
Canine IgGC
(SEQ ID NO: 30)
CNNCPCPGCGLLGGPSVFIFPPKPKDILVTARTPTVTCVVVDLDPENPEV
QISWFVDSKQVQTANTQPREEQSNGTYRVVSVLPIGHQDWLSGKQFKCKV
NNKALPSPIEEIISKTPGQAHQPNVYVLPPSRDEMSKNTVTLTCLVKDFF
PPEIDVEWQSNGQQEPESKYRMTPPQLDEDGSYFLYSKLSVDKSRWQRGD
TFICAVMHEALHNHYTQISLSHSPG
Canine IgGD
(SEQ ID NO: 31)
CISPCPVPESLGGPSVFIFPPKPKDILRITRTPEITCVVLDLGREDPEVQ
ISWFVDGKEVHTAKTQPREQQFNSTYRVVSVLPIEHQDWLTGKEFKCRVN
HIGLPSPIERTISKARGQAHQPSVYVLPPSPKELSSSDTVTLTCLIKDFF
PPEIDVEWQSNGQPEPESKYHTTAPQLDEDGSYFLYSKLSVDKSRWQQGD
TFTCAVMHEALQNHYTDLSLSHSPG

For canines, replacing the human Fc with canine IgGA is preferable to minimize any unwanted immunogenicity in dogs due to the IgGA isotype's lack of Fc(gamma) effector function in dogs (much like the human IgG2 isotype in humans). However, the insulin-Fc fusion protein configuration of SEQ ID NO: 33 comprising the insulin polypeptide of SEQ ID NO: 16, the peptide linker of SEQ ID NO: 25, and the canine IgGA fragment of SEQ ID NO: 28 was highly aggregated with low titers of the desired homodimer as illustrated in Example 20. The protein titer was 22 mg/L and the % homodimer was 24%, yielding a homodimer titer of 5.28 mg/L, which is significantly less than the required homodimer titer of 40 mg/L.

(SEQ ID NO: 33)
FVNQHLCGSDLVEALALVCGERGFFYTDPTGGGPRRGIVEQCCHSICSLY
QLENYCNGGGGAGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG

A further insulin-Fc fusion protein configuration (SEQ ID NO: 35) comprising the canine IgGA Fc fragment of SEQ ID NO: 28, the insulin polypeptide of SEQ ID NO: 4, and the linker of SEQ ID NO: 26 similarly was highly aggregated with low titers of the desired homodimer as illustrated in Example 20. The protein titer was 21 mg/L and the % homodimer was 19%, yielding a homodimer titer of 3.99 mg/L, which is significantly less than the required homodimer titer of 40 mg/L.

(SEQ ID NO: 35)
FVNQHLCGSDLVEALYLVCGERGFFYTDPTGGGPRRGIVEQCCHSICSLY
QLENYCNGGGGSGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG

A further insulin-Fc fusion protein configuration (SEQ ID NO: 37) comprising the canine IgGA Fc fragment of SEQ ID NO: 28, the insulin polypeptide of SEQ ID NO: 23, and the linker of SEQ ID NO: 26 similarly was highly aggregated with low titers of the desired homodimer as illustrated in Example 20. The protein titer was 11 mg/L which is significantly less than the required homodimer titer of 40 mg/L.

(SEQ ID NO: 37)
FVNQHLCGSHLVEALYLVCGERGFFYTPKAGGGPRRGIVEQCCTSICSLY
QLENYCNGGGGSGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG

A further insulin-Fc fusion protein configuration (SEQ ID NO: 39) comprising the canine IgGA Fc fragment of SEQ ID NO: 28, the insulin polypeptide of SEQ ID NO: 22, and the linker of SEQ ID NO: 26 similarly was highly aggregated with low titers of the desired homodimer as illustrated in Example 20. The protein titer was 18 mg/L which is significantly less than the required homodimer titer of 40 mg/L.

(SEQ ID NO: 39)
FVNQHLCGSHLVEALYLVCGERGFFYTPKAAAAAAAKGIVEQCCTSICSL
YQLENYCNGGGGSGGGGRCTDTPPCPVPEPLGGPSVLIFPPKPKDILRIT
RTPEVTCVVLDLGREDPEVQISWFVDGKEVHTAKTQSREQQFNGTYRVVS
VLPIEHQDWLTGKEFKCRVNHIDLPSPIERTISKARGRAHKPSVYVLPPS
PKELSSSDTVSITCLIKDFYPPDIDVEWQSNGQQEPERKHRMTPPQLDED
GSYFLYSKLSVDKSRWQQGDPFTCAVMHETLQNHYTDLSLSHSPG

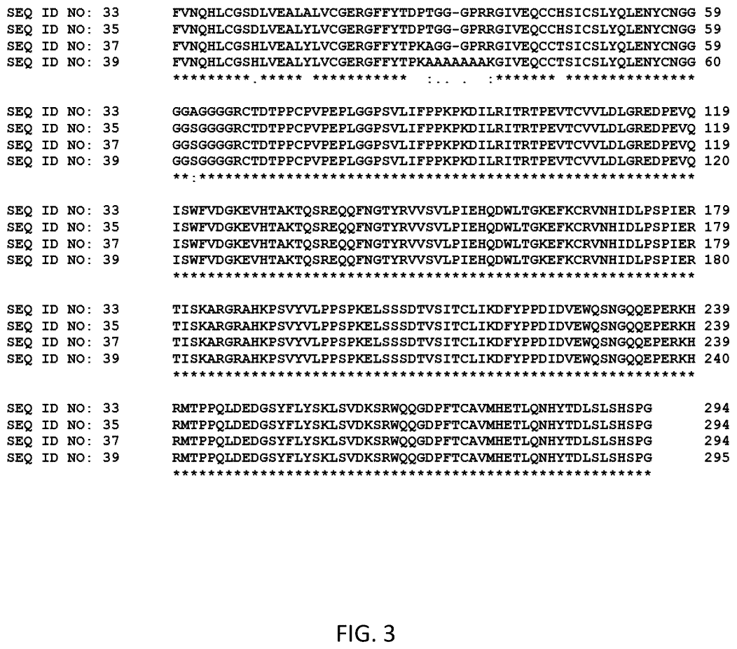

The sequence alignment of the full amino acid sequences of the insulin-Fc fusion proteins SEQ ID NO: 33, SEQ ID NO: 35, SEQ ID NO: 37, and SEQ ID NO: 39 is shown in FIG. 3 (Clustal Omega). Despite mutating the insulin polypeptide and/or the linker, there was no embodiment based on the canine IgGA Fc fragment (SEQ ID NO: 28) with a low enough degree of aggregation and a high enough titer of the desired homodimer.

On the other hand, replacing the canine IgGA Fc fragment (SEQ ID NO: 28) with the canine IgGB Fc fragment (SEQ ID NO: 29) yielded a much less aggregated compound with a comparatively high titer of the desired homodimer. The insulin-Fc fusion protein configuration of SEQ ID NO: 41, comprising the insulin polypeptide of SEQ ID NO: 16, the linker of SEQ ID NO: 25 and the canine IgGB Fc fragment of SEQ ID NO: 29 had a protein yield of 80 mg/L and a % homodimer of 93% resulting in a homodimer titer of 74.7 mg/L, which surpassed the required homodimer titer of 40 mg/L, as illustrated in Example 20.

(SEQ ID NO: 41)
FVNQHLCGSDLVEALALVCGERGFFYTDPTGGGPRRGIVEQCCHSICSLY
QLENYCNGGGGAGGGGDCPKCPAPEMLGGPSVFIFPPKPKDTLLIARTPE
VTCVVVDLDPEDPEVQISWFVDGKQMQTAKTQPREEQFNGTYRVVSVLPI
GHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFL
YSKLSVDKSRWQRGDTFICAVMHEALHNHYTQESLSHSPG

The preference for the canine IgGB Fc fragment over other canine Fc fragments was confirmed in embodiments containing the insulin polypeptide of SEQ ID NO: 16 and the peptide linker of SEQ ID NO: 25, with canine IgGC and IgGD as illustrated in Example 20. The insulin-Fc fusion protein configuration of SEQ ID NO: 43, comprising the insulin polypeptide of SEQ ID NO: 16, the peptide linker of SEQ ID NO: 25 and the canine IgGC Fc fragment of SEQ ID NO: 30 yielded no material at all. The insulin-Fc fusion protein configuration of SEQ ID NO: 45, comprising the insulin polypeptide of SEQ ID NO: 16, the peptide linker of SEQ ID NO: 25 and the canine IgGD Fc fragment of SEQ ID NO: 31 yielded a good protein yield of 134 mg/L, however the material was highly aggregated with a % homodimer of 12.3% and a homodimer titer of 16.5 mg/L, which is significantly less than the required homodimer titer of 40 mg/L. The results are shown in Table 2.

(SEQ ID NO: 43)
FVNQHLCGSDLVEALALVCGERGFFYTDPTGGGPRRGIVEQCCHSICSLY
QLENYCNGGGGAGGGGCNNCPCPGCGLLGGPSVFIFPPKPKDILVTARTP
TVTCVVVDLDPENPEVQISWFVDSKQVQTANTQPREEQSNGTYRVVSVLP
IGHQDWLSGKQFKCKVNNKALPSPIEEIISKTPGQAHQPNVYVLPPSRDE
MSKNTVTLTCLVKDFFPPEIDVEWQSNGQQEPESKYRMTPPQLDEDGSYF
LYSKLSVDKSRWQRGDTFICAVMHEALHNHYTQISLSHSPG
(SEQ ID NO: 45)
FVNQHLCGSDLVEALALVCGERGFFYTDPTGGGPRRGIVEQCCHSICSLY
QLENYCNGGGGAGGGGCISPCPVPESLGGPSVFIFPPKPKDILRITRTPE
ITCVVLDLGREDPEVQISWFVDGKEVHTAKTQPREQQFNSTYRVVSVLPI
EHQDWLTGKEFKCRVNHIGLPSPIERTISKARGQAHQPSVYVLPPSPKEL
SSSDTVTLTCLIKDFFPPEIDVEWQSNGQPEPESKYHTTAPQLDEDGSYF
LYSKLSVDKSRWQQGDTFTCAVMHEALQNHYTDLSLSHSPG

TABLE 2
Homodimer titers for insulin-Fc fusion protein
configurations utilizing canine IgG Fc Fragments

| | | Protein | | Homodimer |
|---|---|---|---|---|
| | Canine | Yield | % | Titer |
| SEQ ID NO: | IgG | (mg/L) | Homodimer | (mg/L) |
| SEQ ID NO: 33 | IgGA | 22 | 24% | 5.28 |
| SEQ ID NO: 35 | IgGA | 21 | 19% | 3.99 |
| SEQ ID NO: 37 | IgGA | 11 | Not tested | ≤11 |
| SEQ ID NO: 39 | IgGA | 18 | Not tested | ≤18 |
| SEQ ID NO: 41 | IgGB | 80 | 93% | 74.4 |
| SEQ ID NO: 43 | IgGC | 0 | Not tested | 0 |
| SEQ ID NO: 45 | IgGD | 134 | 12% | 16.5 |

In vitro insulin receptor binding for the insulin-Fc fusion proteins of SEQ ID NO: 41 and SEQ ID NO: 45 was tested according to the procedure of Example 11. The insulin-Fc fusion protein of SEQ ID NO: 45 demonstrated an IC50 of greater than 5000 nM, as shown in Table 3, indicating that the compound was highly unlikely to show bioactivity in vivo. However, the insulin-Fc fusion protein of SEQ ID NO: 41 demonstrated an IC50 of 28 nM indicating that this sequence was likely to be bioactive in vivo.

TABLE 3
Homodimer titers for sequences utilizing native
canine IgGB, IgGC, and IgGD Fc fragments

| | | | | | IR |
|---|---|---|---|---|---|
| | | Protein | % | Homodimer | Binding, |
| | IgG | Yield | Homo- | Titer | IC50 |
| SEQ ID NO: | Fragment | (mg/L) | dimer | (mg/L) | (nM) |
| SEQ ID NO: 33 | IgGA | 22 | 24% | 5.28 | 2,733 |
| SEQ ID NO: 41 | IgGB | 80 | 93% | 74.4 | 28 |
| SEQ ID NO: 43 | IgGC | 0 | Not tested | 0 | DNM* |
| SEQ ID NO: 45 | IgGD | 134 | 12% | 16.5 | >5000 |

*DNM = Did Not Measure

Given the promising homodimer titer and insulin receptor activity results shown in Table 3, the insulin-Fc fusion protein configuration of SEQ ID NO: 41, comprising the insulin polypeptide of SEQ ID NO: 16, the linker of SEQ ID NO: 25 and the canine IgGB Fc fragment (SEQ ID NO: 29) was bioactive in dogs, exhibiting glucose lowering bioactivity over multiple days (i.e., the NAOC value was greater than 150% FBGL·days·kg/mg).

The preference for insulin-Fc fusion protein configurations comprising the canine IgGB fragment was further evaluated using the insulin polypeptide of SEQ ID NO: 21 and the linker of SEQ ID NO: 27, both of which vary considerably from the insulin polypeptide of SEQ ID NO: 16 and the peptide linker of SEQ ID NO: 25.

(SEQ ID NO: 21)
FVNQHLCGSHLVEALELVCGERGFHYGGGGGGSGGGGGIVEQCCTSTCSL
DQLENYC
(SEQ ID NO: 27)
GGGGGQGGGGQGGGGQGGGGG

Insulin-Fc fusion proteins containing the insulin polypeptide of SEQ ID NO: 21 and the peptide linker of SEQ ID NO: 27 were synthesized using Fc fragments from the canine IgGA (SEQ ID NO: 28), canine IgGB (SEQ ID NO: 29), canine IgGC (SEQ ID NO: 30), or canine IgGD (SEQ ID NO: 31) immunoglobulins.

Using the conventional purification method, only the insulin-Fc fusion configurations comprising canine IgGA (SEQ ID NO: 28) and canine IgGB (SEQ ID NO: 29) showed any appreciable protein yields. However, just like before, the insulin-Fc fusion configuration comprising canine IgGA (SEQ ID NO: 28) was highly aggregated and exhibited low levels of bioactivity, whereas the insulin-Fc fusion configuration comprising canine IgGB (SEQ ID NO: 29) exhibited a low degree of aggregation (i.e. high % homodimer), a high titer of the desired homodimer (i.e., a homodimer titer greater than 40 mg/L), and appreciable levels of long-duration glucose lowering bioactivity in dogs (i.e., the NAOC value was greater than 150% FBGL·days·kg/mg). Using an alternative purification method, the insulin-Fc fusion configuration comprising canine IgGC (SEQ ID NO: 30) was recovered with low degrees of aggregation, however this insulin-Fc fusion protein configuration was minimally bioactive in dogs (i.e., the NAOC value was less than 150% FBGL·days·kg/mg), presumably due to its low affinity for the FcRn receptor. Therefore, with respect to a dog-specific product, the canine IgGB (SEQ ID NO: 28) is the preferred Fc fragment for insulin-Fc fusion protein configurations used in dogs, regardless of the choice of insulin polypeptide or linker.

### Insulin-Fc Fusion Proteins

Provided herein are insulin-Fc fusion proteins comprising an insulin polypeptide, an Fc fragment, and a linker between the insulin polypeptide and the Fc fragment. In embodiments, the insulin polypeptide comprises domains in the following orientation from N- to C-termini: (N-terminus)—B-chain—C-chain—A-chain—(C-terminus). In embodiments, the insulin polypeptide is located on the N-terminal side of the Fc fragment. In embodiments, the fusion protein comprises domains in the following orientation from N- to C-termini: (N-terminus)—insulin polypeptide—linker—Fc fragment—(C-terminus) (e.g., (N-terminus)—B-chain—C-chain—A-chain—linker—Fc fragment—(C-terminus)) as illustrated in FIG. 1.

The repeated dose subcutaneous bioactivity of the insulin-Fc fusion protein of SEQ ID NO: 41 was tested in dogs as per the method described in Example 15. N=3 animals were dosed subcutaneously at day 0, at day 35, and at day 42, and the % FBGL was measured for the 7-day window after each dose according to Example 15. The NAOC and NAOCR were calculated according to the procedure of Example 15 for each repeated subcutaneous injection. As illustrated in Table 4, repeated subcutaneous dosing in dogs unexpectedly revealed a significant decay in bioactivity by the third dose as measured by a significant decrease in the NAOCR (i.e., the NAOC for the third injection was only 0.40, or 40%, of the NAOC for the first injection).

TABLE 4
NAOC per dose and NAOCR for
repeated doses of SEQ ID NO: 41

| Injection | NAOC | NAOCR |
|---|---|---|
| Number of | (% FBGL · | (ratioed |
| SEQ ID NO: 41 | days · kg/mg) | to Week 1) |
| 1 | 330 | 1.0 |
| 2 | 339 | 1.1 |
| 3 | 115 | 0.4 |

Given that the canine IgGB isotype interacts with the canine Fc(gamma) receptor with higher affinities than its canine IgGA counterpart, there may or may not be a risk of unwanted immunogenicity after repeated injections. One method for reducing the Fc(gamma) interaction involves deglycosylating or preventing the glycosylation of the Fc fragment during synthesis in the host cell. Each IgG fragment contains a conserved asparagine (N)-glycosylation site in the CH2 domain of each heavy chain of the Fc region. Herein, the notation used to refer to the conserved N-glycosylation site is “cNg”. One way to remove the attached glycan from a synthesized insulin-Fc fusion protein is to mutate the cNg site to prevent the attachment of glycans altogether during production in the host cell. Herein, the notation used to describe a cNg mutation is cNg-(substituted amino acid). For example, if the asparagine at the cNg site is mutated to serine, this mutation is notated as “cNg-S”.

The absolute position of the cNg site from the N-terminus of the B-chain of the insulin-Fc fusion protein varies depending on the length of the insulin polypeptide, the length of the linker, and any omitted amino acids in the Fc fragment prior to the cNg site. Herein, the notation used to refer to the absolute position of the cNg site in a given insulin-Fc fusion protein sequence (as measured counting from the N-terminus of the B-chain of the insulin-Fc fusion protein) is “NB(number)”. For example, if the cNg site is found at the 138th amino acid position as counted from the N-terminus of the B-chain, the absolute position of this site is referred to as cNg-NB138. As a further example, if the cNg site is found at the 138th amino acid position as counted from the N-terminus of the B-chain, and the asparagine at this site is mutated to serine, this mutation is noted as “cNg- NB138-S”.

Deglycosylation of the Fc fragments of various insulin-Fc fusion protein configurations comprising the canine IgGB Fc fragment of SEQ ID NO: 29 were performed according to Example 25.

In an example, the insulin-Fc fusion protein of SEQ ID NO: 47 comprises the insulin polypeptide of SEQ ID NO: 16, the linker of SEQ ID NO: 25 and the canine IgGB Fc fragment (SEQ ID NO: 29) with cNg-S (in bold).

(SEQ ID NO: 47)
FVNQHLCGSDLVEALALVCGERGFFYTDPTGGGPRRGIVEQCCHSICSLY
QLENYCNGGGGAGGGGDCPKCPAPEMLGGPSVFIFPPKPKDTLLIARTPE
VTCVVVDLDPEDPEVQISWFVDGKQMQTAKTQPREEQFSGTYRVVSVLPI
GHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFL
YSKLSVDKSRWQRGDTFICAVMHEALHNHYTQESLSHSPG

As illustrated in Table 5, the insulin-Fc fusion protein configuration of SEQ ID NO: 47 yielded acceptable homodimer titer and IR binding. In spite of mutating the cNg-NB138 site to S to reduce immunogenicity due to glycosylation, the bioactivity of the insulin-Fc fusion protein demonstrated a significant decay in bioactivity by the third dose as measured by a significant decrease in the NAOCR as illustrated in Example 27.

In an example, the insulin-Fc fusion protein configuration comprises the insulin polypeptide of SEQ ID NO: 24, where B10 is restored to H and A8 is restored to T in an attempt to eliminate anti-drug antibodies targeting the B10D and A8H sites on the insulin polypeptide. The insulin-Fc fusion protein configuration of SEQ ID NO: 49, comprises the insulin polypeptide of SEQ ID NO: 24, the linker of SEQ ID NO: 25 and the canine IgGB Fc fragment (SEQ ID NO: 29) with cNg-Q (B10, A8 and cNg-Q sites in bold).

(SEQ ID NO: 49)
FVNQHLCGSHLVEALALVCGERGFFYTDPTGGGPRRGIVEQCCTSICSLY
QLENYCNGGGGAGGGGDCPKCPAPEMLGGPSVFIFPPKPKDTLLIARTPE
VTCVVVDLDPEDPEVQISWFVDGKQMQTAKTQPREEQFQGTYRVVSVLPI
GHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFL
YSKLSVDKSRWQRGDTFICAVMHEALHNHYTQESLSHSPG

As illustrated in Table 5, the insulin-Fc fusion protein configuration of SEQ ID NO: 49 did not yield acceptable homodimer titer or IR binding. Additionally, in spite of restoring the B10 and A8 immunogenic sites on the insulin-polypeptide and mutating cNg-NB138 site to Q to reduce immunogenicity due to glycosylation, the bioactivity of the insulin-Fc fusion protein demonstrated a significant decay in bioactivity by the third dose as measured by a significant decrease in the NAOCR as illustrated in Example 27.

In a further attempt to reduce unwanted immunogenicity, the first amino acid of the Fc fragment (in the hinge region), the aspartic acid (D), was eliminated. In a preferred embodiment, the insulin-Fc fusion protein configuration of SEQ ID NO: 51, comprises the insulin polypeptide of SEQ ID NO: 16, the linker of SEQ ID NO: 25 and the canine IgGB Fc fragment (SEQ ID NO: 29) with the aspartic acid (D) at the Fc hinge omitted (shown as “_” and the cNg site on the Fc fragment mutated to lysine (K). As shown in Table 5, the insulin-Fc fusion protein configuration of SEQ ID NO: 51 illustrated adequate homodimer titer and acceptable IR binding. Unexpectedly, even with the B10D and A8H immunogenic sites on the insulin-polypeptide, SEQ ID NO: 51 exhibited strong bioactivity in vivo, and no significant decay in bioactivity by the fourth dose, as measured by a significant decrease in the NAOCR.

(SEQ ID NO: 51)
FVNQHLCGSDLVEALALVCGERGFFYTDPTGGGPRRGIVEQCCHSICSLY
QLENYCNGGGGAGGGG_CPKCPAPEMLGGPSVFIFPPKPKDTLLIARTPE
VTCVVVDLDPEDPEVQISWFVDGKQMQTAKTQPREEQFKGTYRVVSVLPI
GHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFL
YSKLSVDKSRWQRGDTFICAVMHEALHNHYTQESLSHSPG

TABLE 5
Homodimer titers and IR binding for insulin-Fc fusion
protein configurations utilizing canine IgGB Fc with cNg mutations

| | | | | | IR |
|---|---|---|---|---|---|
| | | Protein | % | Homodimer | Binding, |
| | cNg | Yield | Homo- | Titer | IC50 |
| SEQ ID NO: | MUTATION | (mg/L) | dimer | (mg/L) | (nM) |
| SEQ ID NO: 47 | S | 76.7 | 98.3% | 75.4 | 145 |
| SEQ ID NO: 49 | Q | 21.4 | 98.7% | 21.1 | >5000 |
| SEQ ID NO: 51 | K | 45.4 | 98.1% | 44.5 | 147 |

In the embodiment comprising the insulin-Fc fusion protein of SEQ ID NO: 51, the nucleic acid sequence (leader sequence in bold) is:

(SEQ ID NO: 52)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggatgccccaagtgccccgctcccgagatgctgggcggacccagcgtg
ttcatcttccctcccaagcccaaggacacactgctgatcgccaggacccc
ggaggtgacctgcgtggtggtggacctggatcccgaagaccccgaggtgc
agatcagctggttcgtggatggaaagcagatgcagaccgccaagacccaa
ccccgggaagagcagttcaaaggcacctacagggtggtgagtgtgttgcc
catcggccaccaggactggctgaaggggaagcaattcacatgcaaggtta
ataacaaggccctgcccagccccatcgagaggaccatcagcaaggccagg
ggccaggcccaccagccatctgtgtacgtgctgcccccatctagggagga
actgagcaagaacacagtcagccttacttgcctgatcaaggacttcttcc
caccggacatagacgtggagtggcagagtaacggccagcaggagcccgag
agcaagtataggaccacaccgccccaactggacgaggacggaagctactt
cctctacagcaaattgagcgttgacaaaagcaggtggcagcgaggcgaca
ccttcatctgcgccgtgatgcacgaggctttgcataaccactacacccag
gagagcctgtcccacagccccggatag.

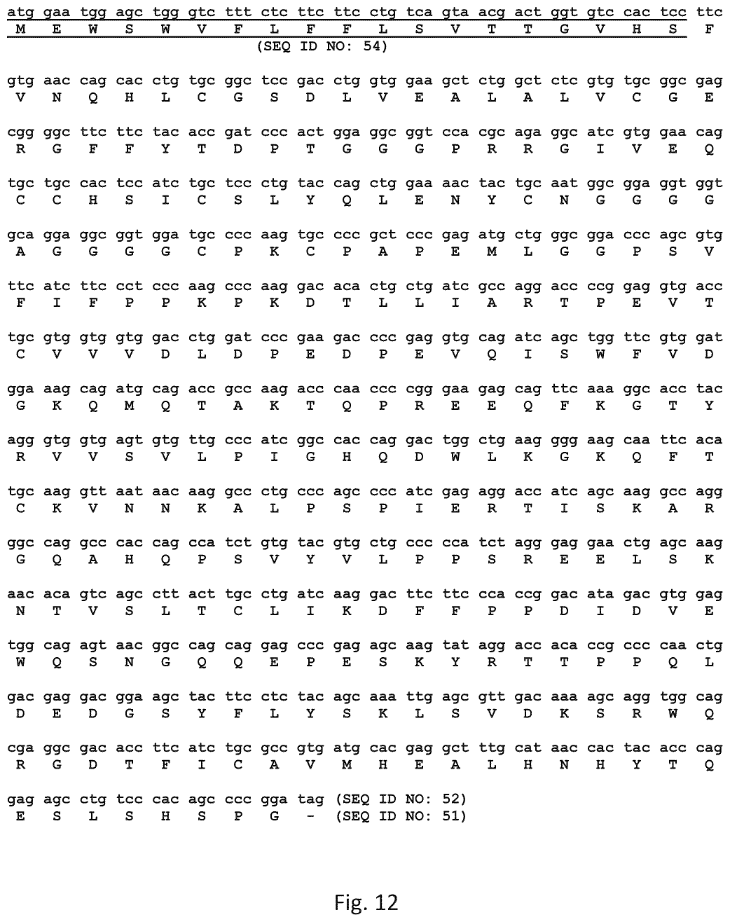

FIG. 12 illustrates an alignment between the amino acid sequence of the insulin-Fc fusion protein of SEQ ID NO: 51 and its corresponding nucleic acid sequence of SEQ ID NO: 52.

In some embodiments, an insulin-Fc fusion protein described herein does not include a leader amino acid sequence at the N-terminus. In other embodiments, an insulin-Fc fusion protein described herein includes a leader sequence, e.g., at the N-terminus. An exemplary leader sequence includes the amino acid sequence MEWSWVFLFFLSVTTGVHS (SEQ ID NO: 54). In some embodiments, an insulin-Fc fusion protein described herein is encoded by a nucleic acid molecule comprising a leader sequence, e.g., for expression (e.g., recombinant expression) in cells (e.g., eukaryotic, e.g., mammalian cells). In certain embodiments, the leader sequence is cleaved off, e.g., in the cell culture, during expression. An exemplary nucleic acid sequence encoding a leader sequence includes the nucleic acid sequence: atggaatggagctgggtctttctcttcttcctgtcagtaacgactggtgtccactcc (SEQ ID NO: 53).

Also disclosed herein are nucleic acid sequences (e.g., cDNA) encoding the insulin-Fc fusion proteins of SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, and SEQ ID NO: 49.

In the embodiment comprising the insulin-Fc fusion protein of SEQ ID NO: 33, the nucleic acid sequence (leader sequence in bold) is:

(SEQ ID NO: 34)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggacgctgcactgacacccctccatgccctgtgcccgagcccctgggt
ggccccagcgtactgatcttcccaccgaaacccaaggacatcctgaggat
cacccgcaccccggaggtgacctgcgtggtgctggacctgggcagggagg
accccgaagtgcaaatcagctggttcgtggacggaaaggaggtgcacacc
gccaagacccaatcaagggagcagcagttcaacggcacctacagggtggt
gagcgtgttgcccatagagcaccaggactggctgaccggcaaggagttca
agtgccgcgtgaaccacattgatctccccagccccatcgagaggactatc
tccaaggcccgagggagggcccacaagcccagtgtatacgtgctgccgcc
ctctccgaaggaactgagctctagcgacaccgtgagcatcacctgcctga
tcaaggacttctaccctcccgacatagacgtagagtggcagagcaacggc
cagcaggagcccgaaaggaagcacaggatgaccccaccccaactggacga
ggacggctcatactttctttatagcaagctgagtgtggacaagagcaggt
ggcagcagggcgaccctttcacttgcgccgtaatgcacgagaccctgcag
aatcactacaccgacctgtcactgagccatagccccggatag

In the embodiment comprising the insulin-Fc fusion protein of SEQ ID NO: 35, the nucleic acid sequence (leader sequence in bold) is:

(SEQ ID NO: 36)
atggaatggagctgggtctttctcttcttcctgtcagtaacgactggtgt
ccactccttcgtgaaccagcacctgtgcggctccgacctggtggaagctc
tgtatctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggttcaggaggcg
gtggacgctgcactgacacccctccatgccctgtgcccgagcccctgggt
ggccccagcgtactgatcttcccaccgaaacccaaggacatcctgaggat
cacccgcaccccggaggtgacctgcgtggtgctggacctgggcagggagg
accccgaagtgcaaatcagctggttcgtggacggaaaggaggtgcacacc
gccaagacccaatcaagggagcagcagttcaacggcacctacagggtggt
gagcgtgttgcccatagagcaccaggactggctgaccggcaaggagttca
agtgccgcgtgaaccacattgatctccccagccccatcgagaggactatc
tccaaggcccgagggagggcccacaagcccagtgtatacgtgctgccgcc
ctctccgaaggaactgagctctagcgacaccgtgagcatcacctgcctga
tcaaggacttctaccctcccgacatagacgtagagtggcagagcaacggc
cagcaggagcccgaaaggaagcacaggatgaccccaccccaactggacga
ggacggctcatactttctttatagcaagctgagtgtggacaagagcaggt
ggcagcagggcgaccctttcacttgcgccgtaatgcacgagaccctgcag
aatcactacaccgacctgtcactgagccatagccccggatag

In the embodiment comprising the insulin-Fc fusion protein of SEQ ID NO: 37, the nucleic acid sequence (leader sequence in bold) is:

(SEQ ID NO: 38)
atggaatggagctgggtctttctcttcttcctgtcagtaacgactggtgt
ccactccttcgtgaaccagcacctgtgcggctcccacctggtggaagctc
tgtatctcgtgtgcggcgagcggggcttcttctacacccccaaggccgga
ggtggacctcgacggggcatcgtggaacagtgctgcacctccatctgctc
cctgtaccagctggaaaactactgcaatggcggaggtggttcaggaggcg
gtggacgctgcactgacacccctccatgccctgtgcccgagcccctgggt
ggccccagcgtactgatcttcccaccgaaacccaaggacatcctgaggat
cacccgcaccccggaggtgacctgcgtggtgctggacctgggcagggagg
accccgaagtgcaaatcagctggttcgtggacggaaaggaggtgcacacc
gccaagacccaatcaagggagcagcagttcaacggcacctacagggtggt
gagcgtgttgcccatagagcaccaggactggctgaccggcaaggagttca
agtgccgcgtgaaccacattgatctccccagccccatcgagaggactatc
tccaaggcccgagggagggcccacaagcccagtgtatacgtgctgccgcc
ctctccgaaggaactgagctctagcgacaccgtgagcatcacctgcctga
tcaaggacttctaccctcccgacatagacgtagagtggcagagcaacggc
cagcaggagcccgaaaggaagcacaggatgaccccaccccaactggacga
ggacggctcatactttctttatagcaagctgagtgtggacaagagcaggt
ggcagcagggcgaccctttcacttgcgccgtaatgcacgagaccctgcag
aatcactacaccgacctgtcactgagccatagccccggatag

In the embodiment comprising the insulin-Fc fusion protein of SEQ ID NO: 39, the nucleic acid sequence (leader sequence in bold) is:

(SEQ ID NO: 40)
atggaatggagctgggtctttctcttcttcctgtcagtaacgactggtgt
ccactccttcgtgaaccagcacctgtgcggctcccacctggtggaagctc
tgtatctcgtgtgcggcgagcggggcttcttctacacccccaaggctgcc
gcagcagctgccgctaaaggcatcgtggaacagtgctgcacctccatctg
ctccctgtaccagctggaaaactactgcaatggcggaggtggttcaggag
gcggtggacgctgcactgacacccctccatgccctgtgcccgagcccctg
ggtggccccagcgtactgatcttcccaccgaaacccaaggacatcctgag
gatcacccgcaccccggaggtgacctgcgtggtgctggacctgggcaggg
aggaccccgaagtgcaaatcagctggttcgtggacggaaaggaggtgcac
accgccaagacccaatcaagggagcagcagttcaacggcacctacagggt
ggtgagcgtgttgcccatagagcaccaggactggctgaccggcaaggagt
tcaagtgccgcgtgaaccacattgatctccccagccccatcgagaggact
atctccaaggcccgagggagggcccacaagcccagtgtatacgtgctgcc
gccctctccgaaggaactgagctctagcgacaccgtgagcatcacctgcc
tgatcaaggacttctaccctcccgacatagacgtagagtggcagagcaac
ggccagcaggagcccgaaaggaagcacaggatgaccccaccccaactgga
cgaggacggctcatactttctttatagcaagctgagtgtggacaagagca
ggtggcagcagggcgaccctttcacttgcgccgtaatgcacgagaccctg
cagaatcactacaccgacctgtcactgagccatagccccggatag

In the embodiment comprising the insulin-Fc fusion protein of SEQ ID NO: 41, the nucleic acid sequence (leader sequence in bold) is:

(SEQ ID NO: 42)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggagactgccccaagtgccccgctcccgagatgctgggggacccagcg
tgttcatcttccctcccaagcccaaggacacactgctgatcgccaggacc
ccggaggtgacctgcgtggtggtggacctggatcccgaagaccccgaggt
gcagatcagctggttcgtggatggaaagcagatgcagaccgccaagaccc
aaccccgggaagagcagttcaacggcacctacagggtggtgagtgtgttg
cccatcggccaccaggactggctgaaggggaagcaattcacatgcaaggt
taataacaaggccctgcccagccccatcgagaggaccatcagcaaggcca
ggggccaggcccaccagccatctgtgtacgtgctgcccccatctagggag
gaactgagcaagaacacagtcagccttacttgcctgatcaaggacttctt
cccaccggacatagacgtggagtggcagagtaacggccagcaggagcccg
agagcaagtataggaccacaccgccccaactggacgaggacggaagctac
ttcctctacagcaaattgagcgttgacaaaagcaggtggcagcgaggcga
caccttcatctgcgccgtgatgcacgaggctttgcataaccactacaccc
aggagagcctgtcccacagccccggatag

In the embodiment comprising the insulin-Fc fusion protein of SEQ ID NO: 43, the nucleic acid sequence (leader sequence in bold) is:

(SEQ ID NO: 44)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggatgcaacaactgcccgtgtccgggatgcggcctcctgggcggaccg
agcgtgttcattttccctcctaagcccaaggacattctggtgaccgccag
gacccccacggtgacctgcgtggtagtagatctcgatcccgaaaacccag
aggtgcaaatcagctggttcgtggactctaagcaagtgcaaaccgccaac
acgcaaccccgcgaggaacagagcaacggcacctacagggtggtgagcgt
gctgcccatcgggcatcaggactggctgagcggcaagcagtttaaatgca
aggttaacaacaaggcactgcccagccccatcgaggagatcatcagcaag
accccgggacaggcccaccagcccaacgtgtacgtccttcctccgagccg
cgacgagatgagcaagaacaccgtgacgctgacctgtttggtgaaggact
tcttcccacccgagatcgacgtggagtggcaaagcaatggccagcaggag
cccgagagcaaatacaggatgaccccaccccaactggatgaggatggcag
ctatttcctctacagcaaattgtccgtggacaaaagcaggtggcagaggg
gcgacaccttcatctgcgccgtcatgcacgaggcccttcacaatcactac
acccagatcagcctgagccactctcccggatag

In the embodiment comprising the insulin-Re fusion protein of SEQ ID NO: 45, the nucleic acid sequence (leader sequence in bold) is:

(SEQ ID NO: 46)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggatgcatcagcccctgccccgtgccggagagcctgggggccctagcg
tgttcatattccctcccaagcccaaggacatcctgaggatcaccaggacc
cccgagatcacctgtgtggtgctggatcttggcagggaagaccccgaagt
ccagatcagctggttcgtggatggcaaggaggtgcacaccgccaagaccc
agccgagggagcagcagttcaactccacctacagggtggtgagcgtgctg
cctatcgagcatcaggactggctgaccggcaaagagttcaagtgcagggt
gaaccacatcggcctgcccagccccatcgagaggaccatcagcaaagcca
ggggccaggcccaccagcccagtgtgtacgtgcttccccctagcccaaag
gaactgagtagcagcgataccgtgaccctgacctgcctgatcaaggactt
tttcccgccagaaatagacgtggagtggcagagcaacggccagccggagc
ccgagagcaaataccacaccaccgcccctcaactggacgaggacgggagc
tacttcctgtatagcaagctgagcgttgacaagagcaggtggcaacaggg
cgacaccttcacctgcgccgtgatgcacgaagctctgcaaaaccactaca
ccgacctgtcactgagccatagccccggatag

In the embodiment comprising the insulin-Fc fusion protein of SEQ ID NO: 47, the nucleic acid sequence (leader sequence in bold) is:

(SEQ ID NO: 48)
atggaatggagctgggtctttctcttcttcctgtcagtaacgactggtgt
ccactccttcgtgaaccagcacctgtgcggctccgacctggtggaagctc
tggctctcgtgtgcggcgagcggggcttcttctacaccgatcccactgga
ggcggtccacgcagaggcatcgtggaacagtgctgccactccatctgctc
cctgtaccagctggaaaactactgcaatggcggaggtggtgcaggaggcg
gtggagactgccccaagtgccccgctcccgagatgctgggcggacccagc
gtgttcatcttccctcccaagcccaaggacacactgctgatcgccaggac
cccggaggtgacctgcgtggtggtggacctggatcccgaagaccccgagg
tgcagatcagctggttcgtggatggaaagcagatgcagaccgccaagacc
caaccccgggaagagcagttcagcggcacctacagggtggtgagtgtgtt
gcccatcggccaccaggactggctgaaggggaagcaattcacatgcaagg
ttaataacaaggccctgcccagccccatcgagaggaccatcagcaaggcc
aggggccaggcccaccagccatctgtgtacgtgctgcccccatctaggga
ggaactgagcaagaacacagtcagccttacttgcctgatcaaggacttct
tcccaccggacatagacgtggagtggcagagtaacggccagcaggagccc
gagagcaagtataggaccacaccgccccaactggacgaggacggaagcta
cttcctctacagcaaattgagcgttgacaaaagcaggtggcagcgaggcg
acaccttcatctgcgccgtgatgcacgaggctttgcataaccactacacc
caggagagcctgtcccacagccccggatag

In the embodiment comprising the insulin-Fc fusion protein of SEQ ID NO: 49, the nucleic acid sequence (leader sequence in bold) is:

(SEQ ID NO: 50)
atggaatggagctgggtctttctcttcttcctgtcagtaacgactggtgt
ccactccttcgtgaaccagcacctgtgcggctcccacctggtggaagctc
tggctctcgtgtgcggcgagcggggcttcttctacaccgatcccactgga
ggcggtccacgcagaggcatcgtggaacagtgctgcacctccatctgctc
cctgtaccagctggaaaactactgcaatggcggaggtggtgcaggaggcg
gtggagactgccccaagtgccccgctcccgagatgctgggcggacccagc
gtgttcatcttccctcccaagcccaaggacacactgctgatcgccaggac
cccggaggtgacctgcgtggtggtggacctggatcccgaagaccccgagg
tgcagatcagctggttcgtggatggaaagcagatgcagaccgccaagacc
caaccccgggaagagcagttccaaggcacctacagggtggtgagtgtgtt
gcccatcggccaccaggactggctgaaggggaagcaattcacatgcaagg
ttaataacaaggccctgcccagccccatcgagaggaccatcagcaaggcc
aggggccaggcccaccagccatctgtgtacgtgctgcccccatctaggga
ggaactgagcaagaacacagtcagccttacttgcctgatcaaggacttct
tcccaccggacatagacgtggagtggcagagtaacggccagcaggagccc
gagagcaagtataggaccacaccgccccaactggacgaggacggaagcta
cttcctctacagcaaattgagcgttgacaaaagcaggtggcagcgaggcg
acaccttcatctgcgccgtgatgcacgaggctttgcataaccactacacc
caggagagcctgtcccacagccccggatag

### Insulin-Fc fusion Protein Production

In embodiments, a fusion protein can be expressed by a cell as described in more detail in the Examples section.

### Expression and Purification

In embodiments, an insulin-Fc fusion protein can be expressed recombinantly, e.g., in a eukaryotic cell, e.g., mammalian cell or non-mammalian cell. Exemplary mammalian cells used for expression include HEK cells (e.g., HEK293 cells) or CHO cells. CHO cells can be subdivided into various strains or subclasses, (e.g. CHO DG44, CHO-M, and CHO-K1), and some of these cell strains may be genetically engineered for optimal use with a particular type of nucleic acid molecule (e.g., a vector comprising DNA) or a particular cell growth media composition as described in the Examples section. In embodiments, cells are transfected with a nucleic acid molecule (e.g., vector) encoding the insulin-Fc fusion protein (e.g., where the entire insulin-Fc fusion protein is encoded by a single nucleic acid molecule). In embodiments, HEK293 cells are transfected with a vector that encodes for the insulin-Fc fusion protein, but only results in temporary expression of the insulin-Fc fusion protein for a period of time (e.g., 3 days, 4 days, 5, days, 7 days, 10 days, 12 days, 14 days, or more) before the host cell stops expressing appreciable levels of the insulin-Fc fusion protein (i.e., transient transfection). HEK293 cells that are transiently transfected with nucleic acid sequences encoding for insulin-Fc fusion proteins often allow for more rapid production of recombinant proteins which facilitates making and screening multiple insulin-Fc fusion protein candidates. In embodiments, CHO cells are transfected with a vector that is permanently incorporated into the host cell DNA and leads to consistent and permanent expression (i.e., stable transfection) of the insulin-Fc fusion protein as long as the cells are cultured appropriately. CHO cells and CHO cell lines that are stably transfected with nucleic acids encoding for insulin-Fc fusion proteins often take longer to develop, but they often produce higher protein yields and are more amenable to manufacturing low cost products (e.g., products for use in the veterinary pharmaceutical market). Cells and cell lines can be cultured using standard methods in the art. In preferred embodiments, HEK cells comprising the cDNA sequence of with SEQ ID NO: 52 is used to express the insulin-Fc fusion protein of SEQ ID NO: 51. In preferred embodiments, CHO cells comprising any the cDNA sequence of SEQ ID NO: 52 is used to express the insulin-Fc fusion protein of SEQ ID NO: 51.

In some embodiments, the insulin-Fc fusion protein is purified or isolated from the cells (e.g., by lysis of the cells). In other embodiments, the insulin-Fc fusion protein is secreted by the cells and purified or isolated from the cell culture media in which the cells were grown. Purification of the insulin-Fc fusion protein can include using column chromatography (e.g., affinity chromatography) or using other separation methods based on differences in size, charge, and/or affinity for certain molecules. In embodiments, purification of the insulin-Fc fusion protein involves selecting or enriching for proteins containing an Fc fragment, e.g., by using Protein A beads or a Protein A column that cause proteins containing an Fc fragment to become bound with high affinity at neutral solution pH to the Protein A covalently conjugated to the Protein A beads. The bound insulin-Fc fusion protein may then be eluted from the Protein A beads by a change in a solution variable (e.g. a decrease in the solution pH). Other separation methods such as ion exchange chromatography and/or gel filtration chromatography can also be employed alternatively or additionally. In embodiments, purification of the insulin-Fc fusion protein further comprises filtering or centrifuging the protein preparation. In embodiments, further purification of the insulin-Fc fusion protein comprises diafiltration, ultrafiltration, and filtration through porous membranes of various sizes, as well as final formulation with excipients.

The purified insulin-Fc fusion protein can be characterized, e.g., for purity, protein yield, structure, and/or activity, using a variety of methods, e.g., absorbance at 280 nm (e.g., to determine protein yield), size exclusion or capillary electrophoresis (e.g., to determine the molecular weight, percent aggregation, and/or purity), mass spectrometry (MS) and/or liquid chromatography (LC-MS) (e.g., to determine purity and/or glycosylation), and/or ELISA (e.g., to determine extent of binding, e.g., affinity, to an anti-insulin antibody). Exemplary methods of characterization are also described in the Examples section.

In embodiments, the protein yield of an insulin-Fc fusion protein after production in transiently transfected HEK cells and protein A purification is greater than 5 mg/L, 10 mg/L, or 20 mg/L. In preferred embodiments, the protein yield of an insulin-Fc fusion protein after production in transiently transfected HEK cells and protein A purification is greater than 40 mg/L (e.g., greater than 60 mg/L, greater than 70 mg/L, greater than 80 mg/L, greater than 90 mg/L, greater than 100 mg/L). In embodiments, the chomodimer of an insulin-Fc fusion protein after production in transiently transfected HEK cells and protein A purification is greater than 70% (e.g., greater than 80%, greater than 85%, greater than 90%, greater than 95%, greater than 96%, greater than 97%, greater than 98%, greater than 99%). In embodiments, the homodimer titer of an insulin-Fc fusion protein after production in transiently transfected HEK cells and protein A purification, calculated as the product between the insulin-Fc fusion protein yield and the chomodimer is greater than 40 mg/L (e.g., greater than 50 mg/L, greater than 60 mg/L, greater than 70 mg/L, greater than 80 mg/L, greater than 90 mg/L, greater than 100 mg/L). Only candidates with a homodimer titer of greater than 40 mg/L were considered useful in the present invention, because experience has demonstrated that homodimer titers less than this level will not likely result in commercial production titers in CHO cells that meet the stringently low manufacturing cost requirements for veterinary products.

In embodiments, the protein yield of an insulin-Fc fusion protein after production in stably transfected CHO cells (e.g., CHO cell lines or CHO cell clones) and protein A purification is greater than 100 mg of insulin-Fc fusion protein per L (e.g. mg/L of culture media). In preferred embodiments, the protein yield of an insulin-Fc fusion protein after production in stably transfected CHO cells (e.g. CHO cell lines or CHO cell clones) and protein A purification is greater than 150 mg insulin-Fc fusion protein/L of culture media (e.g., greater than 200 mg/L, greater than 300 mg/L, greater than 400 mg/L, greater than 500 mg/L, greater than 600 mg/L or more). In embodiments, the % homodimer of an insulin-Fc fusion protein after production in stably transfected CHO cells (e.g. CHO cell lines or CHO cell clones) and protein A purification is greater than 70% (e.g., greater than 80%, greater than 85%, greater than 90%, greater than 95%, greater than 96%, greater than 97%, greater than 98%, greater than 99%). In embodiments, the homodimer titer of an insulin-Fc fusion protein after production in stably transfected CHO cells (e.g. CHO cell lines or CHO cell clones) and protein A purification, calculated as the product between the insulin-Fc fusion protein yield and the % homodimer is greater than 150 mg/L (e.g., greater than 200 mg/L, greater than 300 mg/L, greater than 400 mg/L, greater than 500 mg/L, greater than 600 mg/L or more).

### Functional Features of Insulin-Fc Fusion Proteins

Described herein are methods for interacting with the insulin receptors to lower blood glucose in dogs, wherein the method comprises administering to a subject an insulin-Fc fusion protein, e.g., a fusion protein described herein. In some embodiments, the dog has been diagnosed with diabetes.

In embodiments, an insulin-Fc fusion protein described herein binds to the insulin receptor with an appreciable affinity as measured by the IC50 in the 4° C. IM-9 insulin receptor binding assay described in Example 11 (e.g. IC50 less than 5000 nM, IC50 less than 4000 nM, IC50 less than 3000 nM, IC50 less than 2500 nM). Based on experience, only compounds exhibiting insulin receptor activity IC50 values less than 5000 nM were deemed likely to exhibit bioactivity in the target species. Generally, higher affinity insulin receptor binding (i.e., lower IC50 values) is preferred. However, it is well-known that the clearance of insulin and insulin analogs (e.g., insulin polypeptides described herein) is governed primarily through binding to the insulin receptor followed by insulin receptor internalization and degradation within the cell. Therefore, insulin-Fc fusion proteins with too high of an insulin receptor binding affinity (i.e., too low of an IC50) may be cleared too quickly from circulation resulting in a lower than desired duration of glucose-lowering bioactivity in the target animal.

In embodiments, an insulin-Fc fusion protein described herein is capable of lowering glucose levels (e.g., blood glucose levels) after administration in a subject. In embodiments, the glucose lowering activity of the insulin-Fc fusion protein is greater than that of an insulin reference standard. In some embodiments, the duration of activity of the insulin-Fc fusion protein can be measured by a decrease, e.g., a statistically significant decrease, in fasting blood glucose relative to a pre-dose fasting blood glucose level. In embodiments, the duration of activity of the insulin-Fc fusion protein (e.g., the time during which there is a statistically significant decrease in fasting blood glucose level in a subject relative to a pre-dose level) is longer than about 2 hours. In embodiments, the duration of activity of the insulin-Fc fusion protein (e.g., the time during which there is a statistically significant decrease in blood glucose level in a subject relative to a pre-dose level) is longer than about 6 hours, 9 hours, 12 hours, 18 hours, 1 day, 1.5 days, 2 days, 2.5 days, 3 days, 4 days, 5 days, 6 days, 7 days, or longer. In embodiments, the insulin-Fc fusion protein is long-acting (e.g., has a long half-life, e.g., in serum).

In embodiments, the serum half-life of the insulin-Fc fusion protein in the dog is longer than that of an insulin reference standard or control formulation. In embodiments, the serum half-life of the insulin-Fc fusion protein (e.g., in the blood of a subject upon administration) in the dog is longer than about 2 hours. In embodiments, the serum half-life of the insulin-Fc fusion protein in the dog is about 0.5 days, 1 day, 2 days, or 2.5 days. In preferred embodiments, the serum half-life of the insulin-Fc fusion protein in the dog is about 3 days or longer.

In embodiments, the combination of potency and duration of bioactivity may be quantified by calculating the area over the percent fasting blood glucose (% FBGL) curve normalized to a given dose in mg/kg (NAOC) with units of % FBGL·days·kg/mg. In embodiments, the NAOC of the insulin-Fc fusion protein is greater than 150% FBGL·days·kg/mg (e.g. greater than 200% FBGL·days·kg/mg, greater than 250% FBGL·days·kg/mg or more). Again, based on experience, at NAOC values greater than 150% FBGL·days·kg/mg, the dose requirements in the target species will be sufficiently low so as to achieve an acceptable treatment cost. In embodiments, the NAOC of the insulin-Fc fusion protein must be maintained after repeated dosing in the target species (i.e., the ratio of the NAOC after the third dose to the NAOC after the first dose of the insulin-Fc fusion protein is greater than 0.50 (e.g., greater than 0.60, greater than 0.70. greater than 0.80, greater than 0.90, or more).

In some embodiments, the insulin-Fc fusion protein described herein binds to the Fc(gamma) receptor with an affinity that is lower than that of an insulin-Fc fusion protein reference standard as measured according to Example 12. In some embodiments, the ratio of the Fc(gamma) receptor affinity of the insulin-Fc fusion protein to that of an insulin-Fc fusion protein reference standard is less than 0.50 (e.g. less than 0.40, less than 0.30, less than 0.20).

### Methods of Treatment and Characteristics of Subject Selection

Described herein are methods for treating canine diabetes, the methods comprising the administration of an insulin-Fc fusion protein (e.g., an insulin-Fc fusion protein described herein) to a subject.

In embodiments, a reference standard used in any method described herein comprises a reference treatment or reference therapy. In some embodiments, the reference comprises a standard of care agent for canine diabetes treatment. In some embodiments, the reference standard is a commercially available insulin or insulin analog. In some embodiments, the reference standard comprises a long-lasting insulin, intermediate-lasting insulin, short-lasting insulin, rapid-acting insulin, short-acting, intermediate-acting, long-acting insulin. In some embodiments, the reference standard comprises Vetsulin®, insulin NPH, insulin glargine (Lantus®), or recombinant human insulin.

In embodiments, a reference standard used in any method described herein includes an outcome, e.g., outcome described herein, of a canine diabetes therapy.

In embodiments, a reference standard is a level of a marker (e.g., blood glucose or fructosamine) in the subject prior to initiation of a therapy, e.g., an insulin-Fc fusion protein therapy described herein; where the subject has diabetes. In embodiments, the blood glucose level in a dog is greater than 200 mg/dL (e.g. greater than 250 mg/dL, 300 mg/dL, 350 mg/dL, 400 mg/dL or more) prior to initiation of therapy. In embodiments, the fructosamine level in a dog is greater than 250 micromol/L, 350 micromol/L (e.g. greater than 400 micromol/L, 450 micromol/L, 500 micromol/L, 550 micromol/L, 600 micromol/L, 650 micromol/L, 700 micromol/L, 750 micromol/L or more) prior to initiation of therapy. In embodiments, a reference standard is a measure of the presence of or the progression of or the severity of the disease. In embodiments, a reference standard is a measure of the presence of or the severity of the disease symptoms prior to initiation of a therapy, e.g., an insulin-Fc fusion protein therapy described herein, e.g., where the subject has diabetes.

### Pharmaceutical Compositions and Routes of Administration

Provided herein are pharmaceutical compositions containing an insulin-Fc fusion protein described herein that can be used to lower blood glucose in dogs. The amount and concentration of the insulin-Fc fusion protein in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Formulations of the present disclosure include those suitable for parenteral administration. The phrases “parenteral administration” and “administered parenterally” as used herein means modes of administration other than enteral and topical administration, usually by intravenous or subcutaneous injection.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, e.g., Tween-like surfactants. In some embodiments, the pharmaceutical composition (e.g., as described herein) comprises a Tween-like surfactant, e.g., polysorbate-20, Tween-20 or Tween-80. In some embodiments, the pharmaceutical composition (e.g., as described herein) comprises a Tween-like surfactant, e.g., Tween-80, at a concentration between about 0.001% and about 2%, or between about 0.005% and about 0.1%, or between about 0.01% and about 0.5%.

In some embodiments, the concentration of the insulin-Fc fusion protein in the aqueous carrier is about 3 mg/mL. In some embodiments, the concentration of the insulin-Fc fusion protein in the aqueous carrier is about 6 mg/mL. In some embodiments, the concentration of the insulin-Fc fusion protein in the aqucous carrier is about 8 mg/mL, 9 mg/mL, 10 mg/mL, 12 mg/mL, 15 mg/mL or more.

In some embodiments, the insulin-Fc fusion protein is administered as a bolus, infusion, or an intravenous push. In some embodiments, the fusion protein is administered through syringe injection, pump, pen, needle, or indwelling catheter. In some embodiments, the insulin-Fc fusion protein is administered by a subcutaneous bolus injection. Methods of introduction may also be provided by rechargeable or biodegradable devices. Various slow release polymeric devices have been developed and tested in vivo in recent years for the controlled delivery of drugs, including proteinaccous biopharmaceuticals. A variety of biocompatible polymers (including hydrogels), including both biodegradable and non-degradable polymers, can be used to form an implant for the sustained release of a compound at a particular target site.

### Dosages

Actual dosage levels of the insulin-Fc fusion protein can be varied so as to obtain an amount of the active ingredient that is effective to achieve the desired therapeutic response for a particular dog. The selected dosage level will depend upon a variety of factors including the activity of the particular fusion protein employed, or the ester, salt or amide thereof, the route of administration, the time of administration, the rate of excretion of the particular compound being employed, the duration of the treatment, other drugs, compounds and/or materials used in combination with the particular fusion protein employed, the age, sex, weight, condition, general health and prior medical history of the subject being treated, and like factors well known in the medical arts.

In general, a suitable dose of an insulin-Fc fusion protein will be the amount that is the lowest dose effective to produce a therapeutic effect. Such an effective dose will generally depend upon the factors described above. Generally, intravenous and subcutaneous doses of the insulin-Fc fusion protein for a dog will range from about 0.001 to about 1 mg per kilogram (e.g. mg/kg) of body weight per day, e.g., about 0.001 to 1 mg/kg/day, about 0.01 to 0.1 mg/kg/day, about 0.1 to 1 mg/kg/day, or about 0.01 to 1 mg/kg/day. In still other embodiments, the fusion protein is administered at a dose between 0.025 and 4 mg per kilogram of body weight per week, e.g., between 0.025 and 0.5 mg/kg/weck.

The present disclosure contemplates formulation of the insulin-Fc fusion protein in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### EXAMPLES

The present technology is further illustrated by the following Examples, which should not be construed as limiting in any way.

### General Methods, Assays, and Materials

### Example 1: Synthesis and Methods of Making an Insulin-Fc Fusion Protein in HEK293 Cells

Insulin-Fc fusion proteins were synthesized as follows. A gene sequence of interest was constructed using proprietary software (LakePharma, Belmont, CA) and was cloned into a high expression mammalian vector. HEK293 cells were seeded in a shake flask 24 hours before transfection and were grown using serum-free chemically defined media. A DNA expression construct that encodes the insulin-Fc fusion protein of interest was transiently transfected into a suspension of HEK293 cells using the (LakePharma, Belmont, CA) standard operating procedure for transient transfection. After 20 hours, the cells were counted to determine the viability and viable cell count, and the titer was measured by FortéBio® Octet® (Pall FortéBio LLC, Fremont, CA). Additional readings were taken throughout the transient transfection production run. The culture was harvested on or after day 5.

### Example 2: Synthesis and Methods of Making an Insulin-Fc Fusion Protein in HEK293 Cells

Insulin-Fc fusion proteins are synthesized as follows. A gene sequence of interest is constructed using proprietary software (LakePharma, Belmont, CA) and is cloned into a high expression mammalian vector. HEK293 cells are seeded in a shake flask 24 hours before transfection and are grown using serum-free chemically defined media. A DNA expression construct that encodes the insulin-Fc fusion protein of interest is transiently transfected into a suspension of HEK293 cells using the (LakcPharma, Belmont, CA) standard operating procedure for transient transfection. After 20 hours, the cells are counted to determine the viability and viable cell count, and the titer is measured by FortéBio® Octet® (Pall FortéBio LLC, Fremont, CA). Additional readings are taken throughout the transient transfection production run. The culture is harvested on or after day 5.

### Example 3: Purification of an Insulin-Fc Fusion Protein

Purification of an insulin-Fc fusion protein was performed as follows. Conditioned media supernatants containing the secreted insulin-Fc fusion protein were harvested from the transiently or stably transfected HEK production runs and were clarified by centrifugation. The supernatant containing the desired insulin-Fc fusion protein was run over a Protein A or a Protein G column and eluted using a low pH gradient. Optionally, recovery of the insulin-Fc fusion proteins could be enhanced by reloading of the initial Protein A or Protein G column cluent again onto a second Protein A or Protein G column. Afterwards, the cluted fractions containing the desired protein were pooled and buffer exchanged into 200 mM HEPES, 100 mM NaCl, 50 mM NaOAc, pH 7.0 buffer. A final filtration step was performed using a 0.2 μm membrane filter. The final protein concentration was calculated from the solution optical density at 280 nm. Further optional purification by ion-exchange chromatography (e.g. using an anion exchange bead resin or a cation exchange bead resin), gel filtration chromatography, or other methods was performed as necessary.

### Example 4: Purification of an Insulin-Fc Fusion Protein

Purification of an insulin-Fc fusion protein is performed as follows. Conditioned media supernatants containing the secreted insulin-Fc fusion protein are harvested from the transiently or stably transfected HEK production runs and are clarified by centrifugation. The supernatant containing the desired insulin-Fc fusion protein is run over a Protein A or a Protein G column and eluted using a low pH gradient. Optionally, recovery of the insulin-Fc fusion proteins can be enhanced by reloading of the initial Protein A or Protein G column cluent again onto a second Protein A or Protein G column. Afterwards, the cluted fractions containing the desired protein are pooled and buffer exchanged into 200 mM HEPES, 100 mM NaCl, 50 mM NaOAc, pH 7.0 buffer. A final filtration step is performed using a 0.2 μm membrane filter. The final protein concentration is calculated from the solution optical density at 280 nm. Further optional purification by ion-exchange chromatography (e.g. using an anion exchange bead resin or a cation exchange bead resin), gel filtration chromatography, or other methods is performed as necessary.

### Example 5: Structure Confirmation by Non-Reducing and Reducing CE-SDS

Capillary electrophoresis sodium dodecyl sulfate (CE-SDS) analysis was performed in a LabChip® GXII (Perkin Elmer, Waltham, MA) on a solution of a purified insulin-Fc fusion protein dissolved in 200 mM HEPES, 100 mM NaCl, 50 mM NaOAc, pH 7.0 buffer, and the electropherogram was plotted. Under non-reducing conditions, the sample was run against known molecular weight (MW) protein standards, and the eluting peak represented the ‘apparent’ MW of the insulin-Fc fusion protein homodimer.

Under reducing conditions (e.g. using beta-mercaptoethanol to break disulfide bonds of the insulin-Fc fusion homodimer), the apparent MW of the resulting insulin-Fc fusion protein monomer is compared against half the molecular weight of the insulin-Fc fusion protein homodimer as a way of determining that the structural purity of the insulin-Fc fusion protein is likely to be correct.

### Example 6: Structure Confirmation by Non-Reducing and Reducing CE-SDS

Capillary electrophoresis sodium dodecyl sulfate (CE-SDS) analysis is performed in a LabChip® GXII (Perkin Elmer, Waltham, MA) on a solution of a purified insulin-Fc fusion protein dissolved in 200 mM HEPES, 100 mM NaCl, 50 mM NaOAc, pH 7.0 buffer, and the clectropherogram is plotted. Under non-reducing conditions, the sample is run against known molecular weight (MW) protein standards, and the eluting peak represents the ‘apparent’ MW of the insulin-Fc fusion protein homodimer.

Under reducing conditions (e.g. using beta-mercaptoethanol to break disulfide bonds of the insulin-Fc fusion homodimer), the apparent MW of the resulting insulin-Fc fusion protein monomer is compared against half the molecular weight of the insulin-Fc fusion protein homodimer as a way of determining that the structural purity of the insulin-Fc fusion protein is likely to be correct.

### Example 7: Sequence Identification by LC-MS with Glycan Removal

To obtain an accurate estimate of the insulin-Fc mass via mass spectroscopy (MS), the sample was first treated to remove naturally occurring glycan that might interfere with the MS analysis. 100 μL of a 2.5 mg/mL insulin-Fc fusion protein dissolved in 200 mM HEPES, 100 mM NaCl, 50 mM NaOAc, pH 7.0 buffer solution was first buffer exchanged into 0.1 M Tris, pH 8.0 buffer containing 5 mM EDTA using a Zeba desalting column (Pierce, ThermoFisher Scientific, Waltham, MA). 1.67 μL of PNGase F enzyme (Prozyme N-glycanase) was added to this solution in order to remove N-linked glycan present in the fusion protein (e.g., glycan linked to the side chain of the asparagine located at the cNg-N site), and the mixture was incubated at 37° C. overnight in an incubator. The sample was then analyzed via LC-MS (NovaBioassays, Woburn, MA) resulting in a molecular mass of the molecule which corresponds to the desired homodimer without the glycan. This mass was then further corrected since the enzymatic process used to cleave the glycan from the cNg-asparagine also deaminates the asparagine side chain to form an aspartic acid, and in doing so the enzymatically treated homodimer gains 2 Da overall, corresponding to a mass of 1 Da for each chain present in the homodimer. Therefore, the actual molecular mass is the measured mass minus 2 Da to correct for the enzymatic modification of the insulin-Fc fusion protein structure in the analytical sample.

### Example 8: Sequence Identification by LC-MS with Glycan Removal

To obtain an accurate estimate of the insulin-Fc mass via mass spectroscopy (MS), the sample is first treated to remove naturally occurring glycan that might interfere with the MS analysis. 100 μL of a 2.5 mg/mL insulin-Fc fusion protein dissolved in 200 mM HEPES, 100 mM NaCl, 50 mM NaOAc, pH 7.0 buffer solution is first buffer exchanged into 0.1 M Tris, pH 8.0 buffer containing 5 mM EDTA using a Zeba desalting column (Pierce, ThermoFisher Scientific, Waltham, MA). 1.67 μL of PNGase F enzyme (Prozyme N-glycanase) is added to this solution in order to remove N-linked glycan present in the fusion protein (e.g., glycan linked to the side chain of the asparagine located at the cNg-N site), and the mixture is incubated at 37° C. overnight in an incubator. The sample is then analyzed via LC-MS (NovaBioassays, Woburn, MA) resulting in a molecular mass of the molecule which corresponds to the desired homodimer without the glycan. This mass is then further corrected since the enzymatic process used to cleave the glycan from the cNg-asparagine also deaminates the asparagine side chain to form an aspartic acid, and in doing so the enzymatically treated homodimer gains 2 Da overall, corresponding to a mass of 1 Da for cach chain present in the homodimer. Therefore, the actual molecular mass is the measured mass minus 2 Da to correct for the enzymatic modification of the insulin-Fc fusion protein structure in the analytical sample.

### Example 9: % Homodimer by Size-Exclusion Chromatography

Size-exclusion chromatography (SEC-HPLC) of insulin-Fc fusion proteins was carried out using a Waters 2795HT HPLC (Waters Corporation, Milford, MA) connected to a 2998 Photodiode array at a wavelength of 280 nm. 100 μL or less of a sample containing an insulin-Fc fusion protein of interest was injected into a MAbPac SEC-1, 5 μm, 4×300 mm column (ThermoFisher Scientific, Waltham, MA) operating at a flow rate of 0.2 mL/min and with a mobile phase comprising 50 mM sodium phosphate, 300 mM NaCl, and 0.05% w/v sodium azide, pH 6.2. The MAbPac SEC-1 column operates on the principle of molecular size separation. Therefore, larger soluble insulin-Fc aggregates (e.g. multimers of insulin-Fc fusion protein homodimers) eluted at carlier retention times, and the non-aggregated homodimers cluted at later retention times. In separating the mixture of homodimers from aggregated multimeric homodimers via analytical SEC-HPLC, the purity of the insulin-Fc fusion protein solution in terms of the percentage of non-aggregated homodimer was ascertained.

### Example 10: % Homodimer by Size-Exclusion Chromatography

Size-exclusion chromatography (SEC-HPLC) of insulin-Fc fusion proteins is carried out using a Waters 2795HT HPLC (Waters Corporation, Milford, MA) connected to a 2998 Photodiode array at a wavelength of 280 nm. 100 μL or less of a sample containing an insulin-Fc fusion protein of interest is injected into a MAbPac SEC-1, 5 μm, 4×300 mm column (ThermoFisher Scientific, Waltham, MA) operating at a flow rate of 0.2 mL/min and with a mobile phase comprising 50 mM sodium phosphate, 300 mM NaCl, and 0.05% w/v sodium azide, pH 6.2. The MAbPac SEC-1 column operates on the principle of molecular size separation. Therefore, larger soluble insulin-Fc aggregates (e.g. multimers of insulin-Fc fusion protein homodimers) elute at carlier retention times, and the non-aggregated homodimers clute at later retention times. In separating the mixture of homodimers from aggregated multimeric homodimers via analytical SEC-HPLC, the purity of the insulin-Fc fusion protein solution in terms of the percentage of non-aggregated homodimer is ascertained.

### Example 11: In Vitro IM-9 Insulin Receptor Binding of an Exemplary Insulin-Fc Fusion Protein at 4° C.

Human IM-9 cells (ATTC# CCL-159) that express human insulin receptor were cultured and maintained in complete RPMI 5% FBS medium at 70-80% confluency. Cultures of IM-9 cells were centrifuged at 250×g (˜1000 rpm) for 10 min to pellet the cells. Cells were washed once with HBSS or PBS buffer, resuspended in cold FACS staining medium (HBSS/2mM EDTA/0.1% Na-azide+4% horse serum) to a concentration of 8×106 cells/mL and kept on ice or 4° C. until test solutions were made. The insulin-Fc protein was diluted in FACS buffer in 1:3 serial dilutions as 2× concentrations in 1.2 mL tubes (approx. 60 μL volume of each dilution), and the solutions were kept cold on ice until ready for pipetting.

Biotinylated-RHI was diluted in FACS staining medium to a concentration of 1.25 μg/mL. 40 μL of the serially diluted test compound and 8 μL of 1.25 μg/mL Biotin-RHI were added into each well of a V bottom microtiter plate, mixed by slow vortexing, and placed on ice. 40 μL of an IM-9 cell suspension (8×106 cells/mL) was then added to each well by multichannel pipette, mixed again gently and incubated on ice for 30 min to allow competitive binding on the insulin receptor on IM-9 cells. Cells were then washed twice with 275 μL of ice-cold FACS wash buffer (HBSS/2 mM EDTA/0.1% Na-azide+0.5% horse serum) by centrifuging the V-bottom plate at 3000 rpm for 3 min and aspirating the supernatant. Cells were then resuspended in 40 μL of FACS staining medium containing 1:100 diluted Streptavidin-PE (Life Technologies) for 20 min on ice. Cells were then washed once with 275 μL of ice-cold FACS buffer and finally fixed with 3% paraformaldehyde for 10 min at room temp. Cells were then washed once with 275 μL of ice-cold FACS buffer and resuspended in 250 μl of FACS buffer for analysis.

The V-bottom plates containing cells were then analyzed on a Guava 8-HT flow cytometer (Millipore). Biotinylated-RHI binding to insulin receptor was quantitated by the median fluorescence intensity (MFI) of the cells on the FACS FL-2 channel for each concentration of the test compound. Control wells were labeled only with biotinylated-RHI and were used to calculate the percent (%) inhibition resulting from cach test compound concentration. The % inhibition by test compounds of biotinylated-RHI binding on IM-9 cells was plotted against log concentrations of the test compound, and the resulting IC50 values were calculated using GraphPad Prism (GraphPad Software, La Jolla, CA) for the test compounds. Lower IC50 values of the test compound therefore indicate greater levels of biotinylated-RHI inhibition at lower concentrations indicating stronger binding of the insulin-Fc fusion protein to the insulin receptor. A control compound, such as unlabeled recombinant human insulin (RHI) was also used as an internal standard to generate an RHI IC50 against which a given compound IC50 could be ratioed (IC50(compound)/IC50(RHI)). Lower IC50 ratios have more similar binding to RHI (stronger binding to insulin receptor), while higher IC50 ratios have weaker binding to the insulin receptor relative to RHI.

### Example 12: In Vitro Fc(gamma) Receptor I Binding Affinity Assay

The binding of insulin-Fc fusion proteins to the Fc(gamma) receptor I at pH 7.4 was conducted using an ELISA assay as follows. Since canine Fc(gamma) receptor I was not commercially available, human Fc(gamma) receptor I (i.e., rhFc(gamma) receptor I) was used as a surrogate mammalian receptor. Insulin-Fc compounds were diluted to 10 μg/mL in sodium bicarbonate buffer at pH 9.6 and coated on Maxisorp (Nunc) microtiter plates overnight at 4° C., after which the microplate strips were washed 5 times with PBST (PBS/0.05% Tween-20) buffer and blocked with Superblock blocking reagent (ThermoFisher). Serial dilutions of biotinylated rhFc(gamma) receptor I (recombinant human Fc(gamma)R-I; R&D Systems) were prepared in PBST/10% Superblock buffer from 6000 ng/ml to 8.2 ng/mL and loaded at 100 μL/well onto the microplate strips coated with insulin-Fc fusion protein. The microtiter plate was incubated for 1 hour at room temperature after which the microplate strips were washed 5 times with PBST and then loaded with 100 μL/well of streptavidin-HRP diluted 1:10000 in PBST/10% Superblock buffer. After incubating for 45 min, the microplate strips were washed again 5 times with PBST. TMB was added to reveal the bound Fc(gamma) receptor I proteins and stopped with ELISA stop reagent (Boston Bioproducts). The plate was read in an ELISA plate reader at 450 nm, and the OD values (proportional to the binding of rhFc(gamma) receptor I to insulin-Fc protein) were plotted against log concentrations of rhFc(gamma) receptor I added to each well to generate binding curves using GraphPad Prism software.

### Example 13: Generalized Procedure for Determination of In Vivo Pharmacodynamics (PD) after Single Administration of Insulin Fc-Fusion Proteins in Dogs

Insulin-Fc fusion proteins were assessed for their effects on fasting blood glucose levels as follows. N=1, 2, 3 or more healthy, antibody-naïve, dogs weighing approximately 10-15 kg were used, one for cach insulin-Fc fusion protein. Dogs were also observed twice daily for signs of anaphylaxis, lethargy, distress, pain, etc., and, optionally for some compounds, treatment was continued for an additional three weekly subcutaneous injections or more to observe if the glucose lowering capability of the compounds lessened over time, a key sign of potential induction of neutralizing anti-drug antibodies. On day 0, the dogs received a single injection either via intravenous or subcutaneous administration of a pharmaceutical composition containing an insulin Fc-fusion protein homodimer at a concentration between 1 and 10 mg/mL in a solution of between 10-50 mM sodium hydrogen phosphate, 50-150 mM sodium chloride, 0.005-0.05% v/v Tween-80, and optionally a bacteriostat (e.g. phenol, m-cresol, or methylparaben) at a concentration of between 0.02-1.00 mg/mL, at a solution pH of between 7.0-8.0, at a dose of 0.08-0.80 mg insulin-Fc fusion protein/kg (or approximately equivalent to 1.2-12.3 nmol/kg or approximately equivalent to 0.4-4.0 U/kg insulin equivalent on molar basis). On day 0, blood was collected from a suitable vein immediately prior to injection and at 15, 30, 45, 60, 120, 240, 360, and 480 min and at 1, 2, 3, 4, 5, 6 and 7 days post injection.

For each time point, a minimum of 1 mL of whole blood was collected. A glucose level reading was immediately determined using a glucose meter (ACCU-CHEK® Aviva Plus), which required approximately one drop of blood. Average % fasting blood glucose levels (% FBGL) from day 0 to day 7 were plotted to assess the bioactivity of a given insulin-Fc fusion protein.

### Example 14: Generalized Procedure for Determination of In Vivo Pharmacodynamics (PD) after Single Administration of Insulin Fc-Fusion Proteins in Dogs

Insulin-Fc fusion proteins are assessed for their effects on fasting blood glucose levels as follows. N=1, 2, 3 or more healthy, antibody-naïve, dogs weighing approximately 10-15 kg are used, one for cach insulin-Fc fusion protein. Dogs are also observed twice daily for signs of anaphylaxis, lethargy, distress, pain, etc., and, optionally for some compounds, treatment is continued for an additional three weekly subcutaneous injections or more to observe if the glucose lowering capability of the compounds lessened over time, a key sign of potential induction of neutralizing anti-drug antibodies. On day 0, the dogs receive a single injection either via intravenous or subcutaneous administration of a pharmaceutical composition containing an insulin Fc-fusion protein homodimer at a concentration between 1 and 10 mg/mL in a solution of between 10-50 mM sodium hydrogen phosphate, 50-150 mM sodium chloride, 0.005-0.05% v/v Tween-80, and optionally a bacteriostat (e.g. phenol, m-cresol, or methylparaben) at a concentration of between 0.02-1.00 mg/mL, at a solution pH of between 7.0-8.0, at a dose of 0.08-0.80 mg insulin-Fc fusion protein/kg (or approximately equivalent to 1.2-12.3 nmol/kg or approximately equivalent to 0.4-4.0 U/kg insulin equivalent on molar basis). On day 0, blood is collected from a suitable vein immediately prior to injection and at 15, 30, 45, 60, 120, 240, 360, and 480 min and at 1, 2, 3, 4, 5, 6 and 7 days post injection.

For cach time point, a minimum of 1 mL of whole blood is collected. A glucose level reading is immediately determined using a glucose meter (ACCU-CHEK® Aviva Plus), which requires approximately one drop of blood. Average % fasting blood glucose levels (% FBGL) from day 0 to day 7 are plotted to assess the bioactivity of a given insulin-Fc fusion protein.

### Example 15: Generalized Procedure for Determination of In Vivo Pharmacodynamics (PD) after Repeated Administration of Insulin-Fc Fusion Proteins in Canines

Insulin-Fc fusion proteins were assessed for their effects on blood glucose levels over repeated injections as follows. Healthy, antibody-naïve, dogs weighing approximately between 5 and 20 kg were used, and cach animal was administered doses of an insulin-Fc fusion protein. Animals were observed twice daily for signs of anaphylaxis, lethargy, distress, pain, and other negative side effects, and optionally for some compounds, treatment was continued for up to an additional two to five subcutaneous injections to observe if the glucose lowering capability of the compounds decreased over time, indicating the possible presence of neutralizing anti-drug antibodies in vivo. On day 0, the animals received a single subcutaneous injection of a pharmaceutical composition containing an insulin Fc-fusion protein in a solution of 10-50 mM sodium hydrogen phosphate, 50-150 mM sodium chloride, 0.005-0.05% v/v Tween-80, and optionally a bacteriostat (e.g. phenol, m-cresol, or methylparaben) at a concentration of between 0.02-1.00 mg/mL, at a solution pH of between 7.0-8.0, at a dose of 0.08-0.80 mg insulin-Fc fusion protein/kg (or approximately equivalent to 1.2-12.3 nmol/kg or approximately equivalent to 0.4-4.0 U/kg insulin equivalent on molar basis). On day 0, blood was collected from a suitable vein immediately prior to injection and at 15, 30, 45, 60, 120, 240, 360, and 480 min and at 1, 2, 3, 4, 5, 6 and 7 days post injection.

Subsequent subcutaneous injections were given no more frequently than once-weekly, and in some cases the injections were given at different intervals based on the pharmacodynamics of a given insulin-Fc fusion protein formulation. Subsequent injections for each insulin-Fc fusion protein were adjusted to higher or lower doses, depending on the demonstrated pharmacodynamics of the insulin-Fc fusion protein. For instance, if the dose of a first injection on day 0 was found to be ineffective at lowering blood glucose, the subsequent dose levels of injected insulin-Fc fusion protein were adjusted upward. In a similar manner, if the dose of a first injection on day 0 was found to lower glucose in too strong a manner, then subsequent dose levels of injected insulin-Fc fusion protein were adjusted downward. It was also found that interim doses or final doses could be adjusted in a similar manner as needed. For each dose, blood was collected from a suitable vein just immediately prior to injection and at 15, 30, 45, 60, 120, 240, 360, and 480 min and at 1, 2, 3, 4, 5, 6, 7 days (and optionally 14 days) post injection. For each time point, a minimum of 1 mL of whole blood was collected. A glucose level reading was immediately determined using a glucose meter (ACCU-CHEK® Aviva Plus), which required approximately one drop of blood. Average % fasting blood glucose levels (% FBGL) from throughout the study were plotted against time which allows the bioactivity of a fusion protein to be determined.

To determine the bioactivity of cach dose, an area-over-the-curve (AOC) analysis was conducted as follows. After constructing the % FBGL versus time data, the data was then entered into data analysis software (GraphPad Prism, GraphPad Software, San Diego CA). The software was used to first conduct an area-under-the curve analysis (AUC) to integrate the area under the % FBGL vs. time curve for each dose. To convert the AUC data into the desired AOC data, the following equation was used: AOC=TPA−AUC; where TPA is the total possible area obtained by multiplying cach dose lifetime (e.g., 7 days, 14 days, etc.) by 100% (where 100% represents the y=100% of the % FBGL vs. time curve). For example, given a dose lifetime of 7 days and a calculated AUC of 500% FBGL·days, gives the following for AOC: AOC=(100% FBGL×7 days)−(500% FBGL·days)=200% FBGL·days. The analysis can be performed for each injected dose in a series of injected doses to obtain the AOC values for injection 1, injection 2, injection 3, etc.

As the doses of insulin-Fc fusion protein may vary as previously discussed, it is often more convenient to normalize all calculated AOC values for a given insulin-Fc fusion protein to a particular dose of that insulin-Fc fusion protein. Doing so allows for convenient comparison of the glucose-lowering potency of an insulin-Fc fusion protein across multiple injections, even if the dose levels change across the injections of a given study. Normalized AOC (NAOC) for a given dose is calculated as follows: NAOC=AOC/D with units of % FBGL·days·kg/mg; where D is the actual dose injected into the animal in mg/kg. NAOC values may be calculated for each injection in a series of injections for a given animal and may be averaged across a group of animals receiving the same insulin-Fc fusion protein formulation.

The NAOC ratio (NAOCR) may also be calculated for each injection in a series of injections for a given animal by taking the NAOC values for each injection (e.g. injections 1, 2, 3, . . . N) and dividing cach NAOC for a given injection by the NAOC from injection 1 as follows: NAOCR=(NAOC(Nth injection)/NAOC(injection 1)). By evaluating the NAOCR of a given insulin-Fc homodimer fusion protein formulation for the Nth injection in a series of injections, it is possible to determine whether the in vivo glucose lowering activity of a given insulin-Fc fusion protein has substantially retained its bioactivity over a series of N doses (e.g., NAOCR for the Nth dose of greater than 0.5) or whether the in vivo glucose lowering activity of a given insulin-Fc fusion protein has lost a substantial portion of its potency (e.g., NAOCR of the Nth dose is less than 0.5) over a course of N doses, indicating the potential formation of neutralizing anti-drug antibodies in vivo. In preferred embodiments, the ratio of NAOC following the third subcutaneous injection to the NAOC following the first subcutaneous injection is greater than 0.5 (i.e., the NAOCR of the third subcutaneous injection is greater than 0.5).

### Example 16: Generalized Procedure for Determination of In Vivo Pharmacodynamics (PD) after Repeated Administration of Insulin-Fc Fusion Proteins in Canines

Insulin-Fc fusion proteins are assessed for their effects on blood glucose levels over repeated injections as follows. Healthy, antibody-naïve, dogs weighing approximately between 5 and 20 kg are used, and cach dog is administered doses of an insulin-Fc fusion protein. Dogs are observed twice daily for signs of anaphylaxis, lethargy, distress, pain, and other negative side effects, and optionally for some compounds, treatment is continued for up to an additional two to five subcutaneous injections to observe if the glucose lowering capability of the compounds decreases over time, indicating the possible presence of neutralizing anti-drug antibodies in vivo. On day 0, the animals receive a single subcutaneous injection of a pharmaceutical composition containing an insulin Fc-fusion protein in a solution of 10-50 mM sodium hydrogen phosphate, 50-150 mM sodium chloride, 0.005-0.05% v/v Tween-80, and optionally a bacteriostat (e.g. phenol, m-cresol, or methylparaben) at a concentration of between 0.02-1.00 mg/mL, at a solution pH of between 7.0-8.0, at a dose of 0.08-0.80 mg insulin-Fc fusion protein/kg (or approximately equivalent to 1.2-12.3 nmol/kg or approximately equivalent to 0.4-4.0 U/kg insulin equivalent on molar basis). On day 0, blood is collected from a suitable vein immediately prior to injection and at 15, 30, 45, 60, 120, 240, 360, and 480 min and at 1, 2, 3, 4, 5, 6 and 7 days post injection.

Subsequent subcutaneous injections are given no more frequently than once-weekly, and in some cases the injections are given at different intervals based on the pharmacodynamics of a given insulin-Fc fusion protein formulation. Subsequent injections for each insulin-Fc fusion protein are adjusted to higher or lower doses, depending on the demonstrated pharmacodynamics of the insulin-Fc fusion protein. For instance, if the dose of a first injection on day 0 is found to be ineffective at lowering blood glucose, the subsequent dose levels of injected insulin-Fc fusion protein are adjusted upward. In a similar manner, if the dose of a first injection on day 0 was found to lower glucose in too strong a manner, then subsequent dose levels of injected insulin-Fc fusion protein are adjusted downward. Interim doses or final doses can be adjusted in a similar manner as needed. For each dose, blood is collected from a suitable vein just immediately prior to injection and at 15, 30, 45, 60, 120, 240, 360, and 480 min and at 1, 2, 3, 4, 5, 6, 7 days (and optionally 14 days) post injection. For each time point, a minimum of 1 mL of whole blood is collected. A glucose level reading is immediately determined using a glucose meter (ACCU-CHEK® Aviva Plus), which requires approximately one drop of blood. Average % fasting blood glucose levels (% FBGL) from throughout the study are plotted against time which allows the bioactivity of a fusion protein to be determined.

To determine the bioactivity of each dose, an area-over-the-curve (AOC) analysis is conducted as follows. After constructing the % FBGL versus time data, the data is then entered into data analysis software (GraphPad Prism, GraphPad Software, San Diego CA). The software is used to first conduct an arca-under-the curve analysis (AUC) to integrate the area under the % FBGL vs. time curve for cach dosc. To convert the AUC data into the desired AOC data, the following equation is used: AOC=TPA−AUC; where TPA is the total possible area obtained by multiplying cach dose lifetime (e.g., 7 days, 14 days, etc.) by 100% (where 100% represents the y=100% of the % FBGL vs. time curve). For example, given a dose lifetime of 7 days and a calculated AUC of 500% FBGL·days, gives the following for AOC: AOC=(100% FBGL×7 days)−(500% FBGL·days)=200% FBGL·days. The analysis can be performed for cach injected dose in a series of injected doses to obtain the AOC values for injection 1, injection 2, injection 3, etc.

As the doses of insulin-Fc fusion protein may vary as previously discussed, it is often more convenient to normalize all calculated AOC values for a given insulin-Fc fusion protein to a particular dose of that insulin-Fc fusion protein. Doing so allows for convenient comparison of the glucose-lowering potency of an insulin-Fc fusion protein across multiple injections, even if the dose levels change across the injections of a given study. Normalized AOC (NAOC) for a given dose is calculated as follows: NAOC=AOC/D with units of % FBGL·days·kg/mg; where D is the actual dose injected into the animal in mg/kg. NAOC values may be calculated for each injection in a series of injections for a given animal and may be averaged across a group of animals receiving the same insulin-Fc fusion protein formulation.

The NAOC ratio (NAOCR) may also be calculated for each injection in a series of injections for a given animal by taking the NAOC values for each injection (e.g. injections 1, 2, 3, . . . N) and dividing cach NAOC for a given injection by the NAOC from injection 1 as follows: NAOCR=(NAOC(Nth injection)/NAOC(injection 1)). By evaluating the NAOCR of a given insulin-Fc homodimer fusion protein formulation for the Nth injection in a series of injections, it is possible to determine whether the in vivo glucose lowering activity of a given insulin-Fc fusion protein has substantially retained its bioactivity over a series of N doses (e.g., NAOCR for the Nth dose of greater than 0.5) or whether the in vivo glucose lowering activity of a given insulin-Fc fusion protein has lost a substantial portion of its potency (e.g., NAOCR of the Nth dose is less than 0.5) over a course of N doses, indicating the potential formation of neutralizing anti-drug antibodies in vivo. In preferred embodiments, the ratio of NAOC following the third subcutaneous injection to the NAOC following the first subcutaneous injection is greater than 0.5 (i.e., the NAOCR of the third subcutaneous injection is greater than 0.5).

### Example 17: Assay Protocol for Measuring Anti-Drug Antibodies in Canine Serum

Maxisorp ELISA Plates (Nunc) are coated with the insulin-Fc fusion protein of interest diluted in coating buffer (pH=9.6 Carbonate-Biocarbonate buffer) at 10 μg/mL overnight at 4° C. for measuring ADAs against the test compound. For measuring ADAs against the insulin portion of the insulin-Fc fusion protein containing an Fc fragment of canine IgG origin, plates are coated with purified insulin at 30 μg/mL in coating buffer. Plates are then washed 5× with PBST (PBS+0.05% Tween 20) and blocked for at least 1 hour (or overnight) with SuperBlock blocking solution (ThermoFisher, Waltham MA). For calculating the ADAs in canine IgG units, strips are directly coated with 1:2 serial dilutions of canine IgG (Jackson Immunoresearch Laboratories, West Grove PA) in pH=9.6 Carb-Biocarb coating buffer at concentrations between 300-4.69 ng/ml overnight at 4° C. and used to create a 7-point pseudo-standard curve. The standards strip plates are also washed and blocked with SuperBlock blocking solution for at least 1 hour (or overnight).

Test serum samples are diluted to greater than or equal to 1:100 (typically tested as 1:200) in PBST/SB/20% HS sample dilution buffer (PBS+0.1% Tween 20+10% SuperBlock+20% horse serum) and added to the insulin-Fc fusion protein coated (or RHI coated) strips at 100 μL/well in duplicate. Duplicate strips of canine IgG coated standard strips are also added to each plate and filled with PBST/SB (PBS+0.1% Tween 20+10% SuperBlock) buffer at 100 μL/well. Plates are incubated for 1 hour at RT and then washed 5× with PBST. For detection of ADAs, HRP-conjugated Goat anti-feline IgG F(ab′)2 (anti-feline IgG F(ab′)2 reagent is cross-reacted to canine antibodies; Jackson Immunoresearch Laboratories, West Grove PA), which is diluted in PBST/SB to 1:10000 and added at 100 μL/well to both sample and standard wells and incubated for 45 minutes at RT in dark. Plates are washed 5× with PBST and then one time with deionized water and then developed by adding 100 μL/well TMB substrate (Invitrogen, ThermoFisher Scientific, Waltham MA) for 15-20 minutes at room temperature in the dark. Color development is then stopped by addition of 100 μL/well of ELISA Stop Solution (Boston Bioproducts) and the absorbance is read at 450 nm using a SpectraMax plate reader within 30 minutes. The anti-drug antibody concentration is determined by interpolating the OD values in the 4-PL pseudo-standard curve using SoftMax Pro Software (Molecular Devices, San Jose CA).

To demonstrate the specificity of the detected ADAs, an “inhibition” assay is carried out. In the drug inhibition ADA assay, serum samples are diluted 1:100 in PBST/SB/20% HS buffer and mixed with an equal volume of 300 μg/mL of the relevant therapeutic compound (final sample dilution at 1:200 and final inhibitory compound at 150 μg/mL) and incubated for 30-40 minutes at room temperature to allow anti-drug antibodies to bind the free inhibitor (i.e., the therapeutic compound). After pre-incubation, the samples are added to insulin-Fc fusion protein coated (or RHI coated) strips at 100 μL/well in duplicate. Samples diluted 1:200 in PBST/SB/20% HS buffer without the inhibitory compound are also tested in the sample plates along with duplicate strips of canine IgG coated standards. Remaining steps of the assay procedure are carried out as described above. The ADAs measured in the drug-inhibited wells are matched with the non-inhibited ADA concentrations to assess the specificity of the ADAs. If significant inhibition of ADA signals is observed in the drug-inhibited wells, this means the ADAs are specific to the therapeutic compound.

### Example 18: Assay Procedure for Immunogenic Epitope Identification

Maxisorp ELISA microplates (Nunc) are coated with a library of insulin-Fc fusion protein homodimer compounds with known amino acid sequences, and the coated plates are blocked in a similar manner as described in the anti-drug antibody ELISA assay Example 17, except that cach compound in the library is coated on a separate individual strip of ELISA microplate wells. The compounds in the library comprise a range of insulin-Fc fusion proteins with different insulin polypeptide amino acid compositions, including various B-chain, C-chain, and A-chain amino acid mutations, different linker compositions, and different Fc fragment compositions, including some of human origin. Separately, some plate strip wells are directly coated with 1:2 serial dilutions of canine IgG (Jackson Immunoresearch Laboratories, West Grove PA) for calculating the anti-drug antibodies (ADA) in canine IgG units, respectively, as described in Example 17.

Serum obtained from individual dogs receiving repeated doses of an insulin-Fc fusion protein is first screened on the anti-drug antibody ELISA assay (Example 17). Serum samples demonstrating moderate or high positivity (e.g. moderate or high titers of antibodies) on the assay of Example 17 are serially diluted (1:200 to 1:8000) in PBST/SB/20% HS sample dilution buffer (PBS+0.1% Tween 20+10% SuperBlock+20% horse serum) and added to the plates coated with the library of insulin-Fc fusion protein compounds for 1 hour at RT. Following incubation, the plates are washed 5 times with PBST. For detection of canine antibodies capable of cross-reacting to the coated compound library, HRP conjugated goat anti-feline IgG F(ab′)2 (Jackson Immunoresearch Laboratories, West Grove PA), which is cross-reactive to canine IgGs, is diluted in PBST/SB to 1:10000 and added at 100 μL/well to both sample and standard wells and incubated for 45 min at RT in the dark. Plates are washed 5 times with PBST, once with deionized water, and developed by the adding 100 μL/well TMB substrate (Invitrogen, ThermoFisher Scientific, Waltham MA) for 15-20 min at RT in the dark. Color development is then stopped by addition of 100 μL/well of ELISA Stop Solution (Boston Bioproducts, Ashland MA) and absorbance is read at 450 nm using a SpectraMax plate reader within 30 min. Anti-compound cross-reactive antibody concentrations present in the serum samples are determined by interpolating the OD values in the 4-PL pseudo-standard curve against the directly coated canine IgG antibody controls using SoftMax Pro Software (Molecular Devices, San Jose CA).

By correlating the resulting antibody concentrations from the assay with the known amino acid compositions of the coated insulin-Fc fusion protein library, one can determine whether particular amino acid mutations or epitopes are responsible for causing none, some, most, or all of the total antibody signal on the assay, indicating no binding, weak binding, or strong binding to various insulin-Fc fusion protein homodimers. The mutations or epitopes responsible for moderate or strong binding are herein referred to as immunogenic “hot spots”.

### Example 19: Design Process for Obtaining Insulin-Fc fusion Proteins with High Homodimer Titers and Acceptable Levels of Acute and Repeated Dose Bioactivity in the Target Species

The process for meeting the design goals described in the Detailed Description of the Invention comprised the following steps. First, the insulin polypeptide of SEQ ID NO: 15 or SEQ ID NO: 8 was combined with a species-specific Fc fragment of a particular IgG isotype and a linker such that the resulting insulin-Fc fusion protein was most likely to yield a long-acting bioactivity product with minimal immunogenicity (e.g., a species-specific IgG isotype was chosen with minimal Fc(gamma)receptor I binding). The DNA sequence coding for the desired fusion protein was prepared, cloned into a vector (LakePharma, San Carlos, CA), and the vector was then used to transiently transfect HEK cells according to the procedure described in Example 1. The insulin-Fc fusion protein was then purified according to Examples 3 and the overall protein yield and chomodimer measured according to Example 9. Only candidates with a homodimer titer of greater than 40 mg/L were considered acceptable, because titers less than this level are not likely to result in commercial production titers that meet the stringently low manufacturing cost requirements for veterinary products. Selected insulin-Fc fusion proteins were then screened for indicators of bioactivity through in vitro insulin receptor binding studies as described in Example 11. Based on experience, only compounds that exhibited IR activity IC50 values less than 5000 nM were deemed likely to exhibit bioactivity in the target species. Although the in vitro IR IC50 value is a useful qualitative screening tool, it utilizes human IM-9 cells which express the human insulin receptor and therefore it may not capture some of the small differences in affinity between the canine IR and the human IR. Furthermore, factors other than insulin receptor binding may influence a compound's bioactivity in vivo (e.g., affinity for canine FcRn to allow for extended pharmacokinetic elimination half-lives in vivo). Therefore, selected insulin-Fc fusion proteins that were acceptable from a manufacturing and IR activity IC50 value standpoint were further screened for bioactivity in the animal of interest (e.g., dog or cat) to screen out any materials with less than the desired potency and/or duration of bioactivity (e.g., NAOC of less than 150% FBGL·days·kg/mg). Again, based on experience, at NAOC values of greater than 150% FBGL·days·kg/mg, the dose requirements in the target species will be sufficiently low so as to reach an acceptable treatment cost. Lastly, an additional evaluation criterion was added which is mentioned rarely if ever in the art. As discussed in more detail in the Examples below, many insulin-Fc fusion protein embodiments that exhibit acceptable NAOC levels in the target species after the first dose, unexpectedly fail to maintain that level of bioactivity after repeated doses. Furthermore, in most cases the reduction in repeated dose bioactivity in the target species is correlated with the development of neutralizing anti-drug antibodies. This propensity to generate anti-drug antibodies and the failure to maintain activity render such insulin-Fc fusion proteins impractical for use in treating a chronic disease such as canine diabetes. Therefore, only the insulin-Fc fusions proteins exhibiting acceptable levels of repeated dose bioactivity (e.g., NAOCR values greater than 0.50 for the third dose relative to the first dose) with minimal levels of anti-drug antibodies were deemed acceptable for use in the present invention.

### Results—Insulin-Fc Fusion Proteins Comprising a Canine Fc Fragment

### Example 20: Canine Insulin-Fc Fusion Protein Configurations Comprising the Canine Fc IgGA, IgGC and IgGD Isotypes

An attempt was made to produce insulin-Fc fusion proteins comprising the insulin polypeptide sequences of SEQ ID NO: 15, SEQ ID NO: 16, SEQ ID NO: 23, and SEQ ID NO: 22, and the Fc fragment of the canine IgGA isotype (SEQ ID NO: 28), the canine IgGC isotype (SEQ ID NO: 30) or the canine IgGD isotype (SEQ ID NO: 31) using the peptide linker of SEQ ID NO: 26 or SEQ ID NO: 25. The full amino acid sequences for the resulting insulin-Fc fusion protein configurations are as follows:

(SEQ ID NO: 33)
FVNQHLCGSDLVEALALVCGERGFFYTDPTGGGPRRGIVEQCCHSICSLY
QLENYCNGGGGAGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG
(SEQ ID NO: 35)
FVNQHLCGSDLVEALYLVCGERGFFYTDPTGGGPRRGIVEQCCHSICSLY
QLENYCNGGGGSGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG
(SEQ ID NO: 37)
FVNQHLCGSHLVEALYLVCGERGFFYTPKAGGGPRRGIVEQCCTSICSLY
QLENYCNGGGGSGGGGRCTDTPPCPVPEPLGGPSVLIFPPKPKDILRITR
TPEVTCVVLDLGREDPEVQISWFVDGKEVHTAKTQSREQQFNGTYRVVSV
LPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDG
SYFLYSKLSVDKSRWQQGDPFTCAVMHETLQNHYTDLSLSHSPG
(SEQ ID NO: 39)
FVNQHLCGSHLVEALYLVCGERGFFYTPKAAAAAAAKGIVEQCCTSICSL
YQLENYCNGGGGSGGGGRCTDTPPCPVPEPLGGPSVLIFPPKPKDILRIT
RTPEVTCVVLDLGREDPEVQISWFVDGKEVHTAKTQSREQQFNGTYRVVS
VLPIEHQDWLTGKEFKCRVNHIDLPSPIERTISKARGRAHKPSVYVLPPS
PKELSSSDTVSITCLIKDFYPPDIDVEWQSNGQQEPERKHRMTPPQLDED
GSYFLYSKLSVDKSRWQQGDPFTCAVMHETLQNHYTDLSLSHSPG

The insulin-Fc fusion protein configuration of SEQ ID NO: 43, comprising the insulin polypeptide of SEQ ID NO: 16, the peptide linker of SEQ ID NO: 25 and the canine IgGC Fc fragment of SEQ ID NO: 30 yielded no material at all. The insulin-Fc fusion protein configuration of SEQ ID NO: 45, comprising the insulin polypeptide of SEQ ID NO: 16, the peptide linker of SEQ ID NO: 25 and the canine IgGD Fc fragment of SEQ ID NO: 31 yielded a good protein yield of 134 mg/L, however the material was highly aggregated with a % homodimer of 12.3% and a homodimer titer of 16.5 mg/L, which is significantly less than the required homodimer titer of 40 mg/L.

(SEQ ID NO: 43)
FVNQHLCGSDLVEALALVCGERGFFYTDPTGGGPRRGIVEQCCHSICSLY
QLENYCNGGGGAGGGGCNNCPCPGCGLLGGPSVFIFPPKPKDILVTARTP
TVTCVVVDLDPENPEVQISWFVDSKQVQTANTQPREEQSNGTYRVVSVLP
IGHQDWLSGKQFKCKVNNKALPSPIEEIISKTPGQAHQPNVYVLPPSRDE
MSKNTVTLTCLVKDFFPPEIDVEWQSNGQQEPESKYRMTPPQLDEDGSYF
LYSKLSVDKSRWQRGDTFICAVMHEALHNHYTQISLSHSPG
(SEQ ID NO: 45)
FVNQHLCGSDLVEALALVCGERGFFYTDPTGGGPRRGIVEQCCHSICSLY
QLENYCNGGGGAGGGGCISPCPVPESLGGPSVFIFPPKPKDILRITRTPE
ITCVVLDLGREDPEVQISWFVDGKEVHTAKTQPREQQFNSTYRVVSVLPI
EHQDWLTGKEFKCRVNHIGLPSPIERTISKARGQAHQPSVYVLPPSPKEL
SSSDTVTLTCLIKDFFPPEIDVEWQSNGQPEPESKYHTTAPQLDEDGSYF
LYSKLSVDKSRWQQGDTFTCAVMHEALQNHYTDLSLSHSPG

The insulin-Fc fusion protein configurations of SEQ ID NO: 33, SEQ ID NO: 35, SEQ ID NO: 37, SEQ ID NO: 39, SEQ ID NO: 43 and SEQ ID NO: 45 were synthesized in HEK cells according to Example 1 and purified according to Example 3. The structure of the insulin-Fc fusion protein was confirmed according to Example 5 by non-reducing and reducing CE-SDS, and the sequence was further identified by LC-MS with glycan removal according to Example 7. The % homodimer was measured by size-exclusion chromatography according to Example 9. The results are given in Table 6. In summary, despite mutating the insulin polypeptide and/or the linker, there was no embodiment based on the canine IgGA Fc fragment (SEQ ID NO: 28) with a low enough degree of aggregation and a high enough titer of the desired homodimer.

TABLE 6
Homodimer titers for insulin-Fc fusion protein
configurations utilizing canine IgG Fc Fragments

| | | Protein | | Homodimer |
|---|---|---|---|---|
| | Canine | Yield | % | Titer |
| SEQ ID NO: | IgG | (mg/L) | Homodimer | (mg/L) |
| SEQ ID NO: 33 | IgGA | 22 | 24% | 5.28 |
| SEQ ID NO: 35 | IgGA | 21 | 19% | 3.99 |
| SEQ ID NO: 37 | IgGA | 11 | Not tested | ≤11 |
| SEQ ID NO: 39 | IgGA | 18 | Not tested | ≤18 |
| SEQ ID NO: 43 | IgGC | 0 | Not tested | 0 |
| SEQ ID NO: 45 | IgGD | 134 | 12% | 16.5 |

### Example 21: Canine Insulin-Fc Fusion Protein Configurations Comprising the Canine Fc IgGB Isotype

On the other hand, replacing the canine IgGA Fc fragment (SEQ ID NO: 28) with the canine IgGB Fc fragment (SEQ ID NO: 29) yielded a much less aggregated compound with a comparatively high titer of the desired homodimer. The insulin-Fc fusion protein configuration of SEQ ID NO: 41, comprising the insulin polypeptide of SEQ ID NO: 16, the linker of SEQ ID NO: 25 and the canine IgGB Fc fragment of SEQ ID NO: 29 had a protein yield of 80 mg/L and a % homodimer of 93% resulting in a homodimer titer of 74.7 mg/L, which surpassed the required homodimer titer of 40 mg/L.

(SEQ ID NO: 41)
FVNQHLCGSDLVEALALVCGERGFFYTDPTGGGPRRGIVEQCCHSICSLY
QLENYCNGGGGAGGGGDCPKCPAPEMLGGPSVFIFPPKPKDTLLIARTPE
VTCVVVDLDPEDPEVQISWFVDGKQMQTAKTQPREEQFNGTYRVVSVLPI
GHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFL
YSKLSVDKSRWQRGDTFICAVMHEALHNHYTQESLSHSPG

### Example 22: In Vitro IM-9 Insulin Receptor Binding of an Exemplary Insulin-Fc Fusion Protein at 4° C.

In vitro insulin receptor binding for the insulin-Fc fusion protein configurations of SEQ ID NO:25 (IgGA), SEQ ID NO: 41 (IgGB), SEQ ID NO: 43 (IgGC) and SEQ ID NO: 45 (IgGD) were tested according to the procedure of Example 11. As shown in Table 7, the insulin-Fc fusion protein of SEQ ID NO: 33 demonstrated in IR binding IC50 of 2733 and the insulin-Fc fusion protein of SEQ ID NO: 45 demonstrated an IR binding IC50 of greater than 5000 nM, indicating that neither insulin-Fc fusion protein configuration was highly unlikely to show bioactivity in vivo. The insulin-Fc fusion protein of SEQ ID NO: 43 had no yield and so the IR binding IC50 was not measure. However, the insulin-Fc fusion protein of SEQ ID NO: 41 demonstrated an IC50 of 28 nM indicating that this sequence was likely to be bioactive in vivo.

TABLE 7
Homodimer titers for sequences utilizing native
canine IgGB, IgGC, and IgGD Fc fragments

| | | | | | IR |
|---|---|---|---|---|---|
| | | Protein | % | Homodimer | Binding, |
| | IgG | Yield | Homo- | Titer | IC50 |
| SEQ ID NO: | Fragment | (mg/L) | dimer | (mg/L) | (nM) |
| SEQ ID NO: 33 | IgGA | 22 | 24% | 5.28 | 2,733 |
| SEQ ID NO: 41 | IgGB | 80 | 93% | 74.4 | 28 |
| SEQ ID NO: 43 | IgGC | 0 | Not tested | 0 | DNM* |
| SEQ ID NO: 45 | IgGD | 134 | 12% | 16.5 | >5000 |

*DNM = Did Not Measure

### Example 23: In Vivo Efficacy of an Insulin-Fc fusion Protein Comprising the Insulin Polypeptide of SEQ ID NO: 16 with a Canine IgGB Isotype Fc Fragment

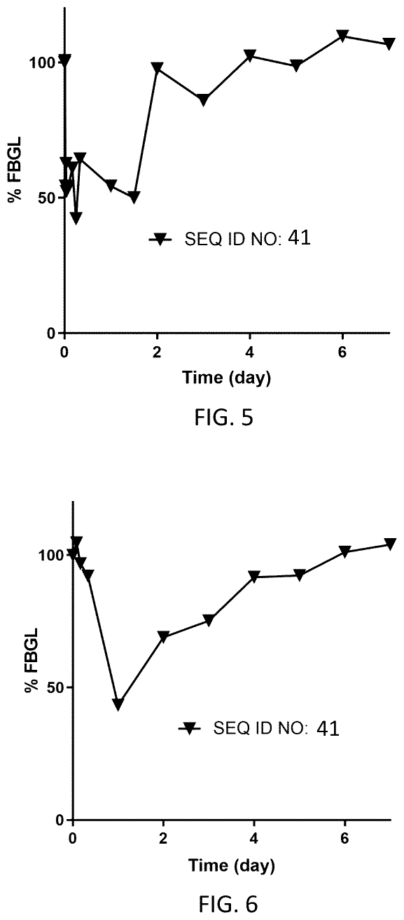

Given the promising homodimer titer and insulin receptor activity results in Examples 17a and 17b, the insulin-Fc fusion protein of SEQ ID NO: 41 was tested for in vivo bioactivity according to Example 13 following an intravenous injection in each of N=3 healthy, antibody-naïve, beagle dogs weighing approximately 10 kg. In a separate experiment, the compound was administered subcutaneously to N=3 naïve beagle dogs. FIG. 5 shows the % FBGL versus time for a single intravenous administration of the insulin-Fc fusion protein of SEQ ID NO: 41, and FIG. 6 shows the % FBGL vs. time for a single subcutaneous administration of the insulin-Fc fusion protein of SEQ ID NO: 41, both of which demonstrate that the insulin-Fc fusion protein of SEQ ID NO: 41 is significantly bioactive in dogs.

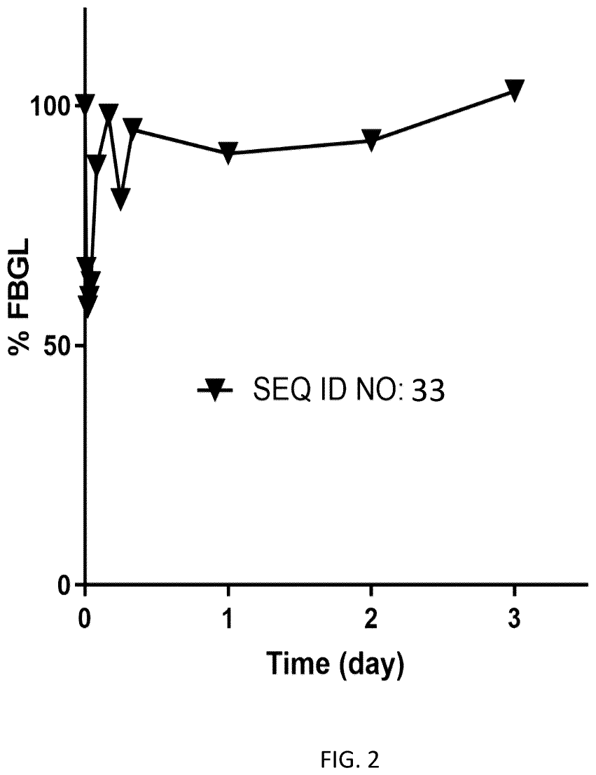

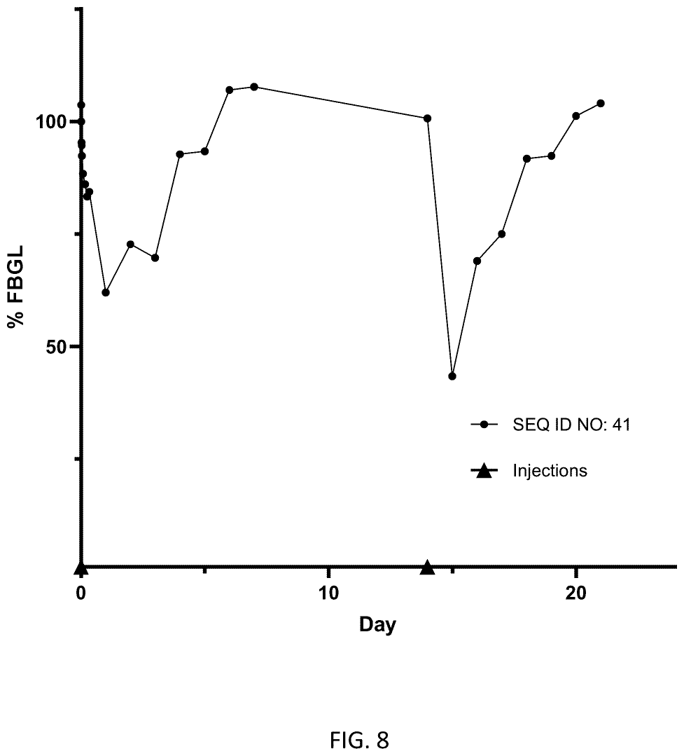

The NAOC was calculated according to the procedure of Example 15 to determine the relative bioactivity and duration of action of the insulin-Fc fusion protein. The NAOC of the insulin-Fc fusion protein of SEQ ID NO: 41 injected intravenously was 399% FBGL·days·kg/mg which was 3.8 times the NAOC of the insulin-Fc fusion protein of SEQ ID NO: 33 injected intravenously, illustrating significantly increased bioactivity for the insulin-Fc fusion protein comprising the canine IgGB Fc fragment versus the insulin-Fc fusion protein comprising the canine IgGA Fc fragment. The in vivo bioactivity of SEQ ID NO: 33 is illustrated in FIG. 2. The NAOC of the insulin-Fc fusion protein of SEQ ID NO: 41 injected subcutaneously was 366% FBGL·days·kg/mg, demonstrating a level of bioactivity via subcutaneous administration that is similar to that obtained via intravenous administration. The in vivo bioactivity of SEQ ID NO: 41 across two doses is illustrated in FIG. 8.

### Example 24: In Vivo Immunogenicity Screening after Repeated Subcutaneous Doses of the Insulin-Fc Fusion Protein Comprising the Insulin Polypeptide of SEQ ID NO: 41 with a Canine IgGB Isotype Fc Fragment

Next, the repeated dose subcutaneous bioactivity of the insulin-Fc fusion protein of SEQ ID NO: 41 was tested in dogs as per the method described in Example 15. N=3 animals were dosed subcutaneously at day 0, at day 35, and at day 42, and the % FBGL was measured for the 7-day window after each dose according to Example 15. The NAOC and NAOCR were calculated according to the procedure of Example 15 for each repeated subcutaneous injection. As illustrated in Table 8, repeated subcutaneous dosing in dogs unexpectedly revealed a significant decay in bioactivity by the third dose as measured by a significant decrease in the NAOCR (i.e., the NAOC for the third injection was only 0.40, or 40%, of the NAOC for the first injection).

TABLE 8
NAOC per dose and NAOCR
for repeated doses of SEQ ID NO: 42

| Injection | NAOC | NAOCR |
|---|---|---|
| Number of | (% FBGL · | (ratioed |
| SEQ ID NO: 41 | days · kg/mg) | to Week 1) |
| 1 | 330 | 1.0 |
| 2 | 339 | 1.1 |
| 3 | 115 | 0.4 |

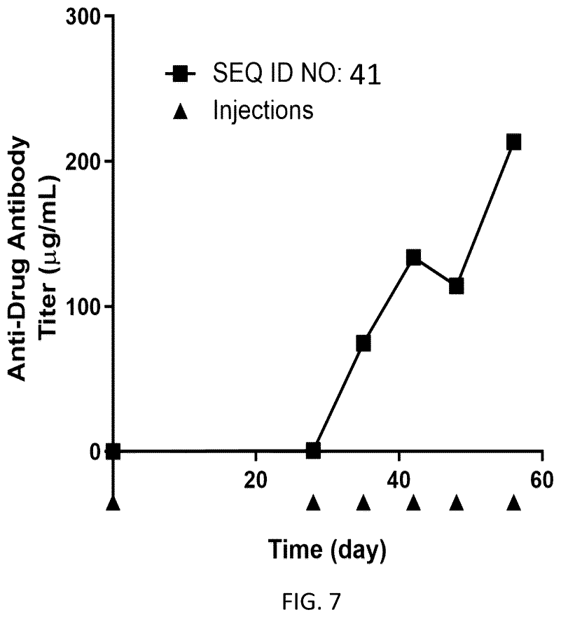

Without being bound to any particular explanation, it was postulated that the cause of the significant reduction in bioactivity of the insulin-Fc fusion protein of SEQ ID NO: 41 after the third repeated subcutaneous dose in dogs was due to the development of anti-drug antibodies that neutralized its biological activity. Anti-drug antibodies may be directed against the insulin polypeptide, linker, or Fc-fragment portions of an insulin-Fc fusion protein. The immunogenic response manifests as interactions between antigen presenting cells, T-helper cells, B-cells, and their associated cytokines, which may lead to the production of endogenous antibodies against the drug (e.g. anti-drug antibodies). Binding antibodies are all isotypes capable of binding the insulin-Fc fusion protein, and these may be detected in an immunoassay as described in Example 17. Neutralizing antibodies that inhibit functional activity of the insulin-Fc fusion protein are generally directed against an epitope that is required for bioactivity. To assess whether this was the case, serum that was collected prior to the administration of each dose and at the end of the experiment described in Examples 11 was tested to quantify the levels of anti-drug antibodies according to Example 17. As shown in FIG. 7, levels of anti-drug antibodies did indeed increase with multiple subcutaneous administrations of the compound, indicating that the generation of neutralizing anti-drug antibodies were the likely cause for the reduction in the NAOCR after the third injection of the insulin Fc-fusion protein of SEQ ID NO: 41.

### Example 25: Non-Glycosylated Insulin-Fc Fusion Protein Comprising the Insulin Polypeptide of SEQ ID NO: 16 with Canine IgGB Isotype Fc Fragments to Reduce the Potential Risk of Immunogenicity

As shown in Example 21, Example 22 and Example 23, the insulin-Fc fusion protein of SEQ ID NO: 41 showed acceptable % homodimer content, homodimer titer, and bioactivity in dogs; however, its use for a chronic disease such as diabetes is compromised by the reduction in bioactivity and generation of anti-drug antibodies (Example 24) with repeated subcutaneous dosing. Without being bound to any particular theory, one possible cause of the generation of anti-drug antibodies and the reduction in bioactivity is the increased interaction of the canine IgGB Fc fragment with various receptors of the canine immune system (e.g. Fc(gamma) receptors, e.g. Fc(gamma)RI). Nevertheless, the canine IgGB isotype was the only one of the four canine IgG isotypes that, when used for the Fc fragment, resulted in an insulin-Fc fusion protein meeting the manufacturability and single-dose bioactivity design goals (Example 19). As described in the Detailed Description of the Invention, one method for reducing the Fc(gamma) interaction involves mutating the Fc fragment cNg site to prevent glycosylation during synthesis in the host cell.

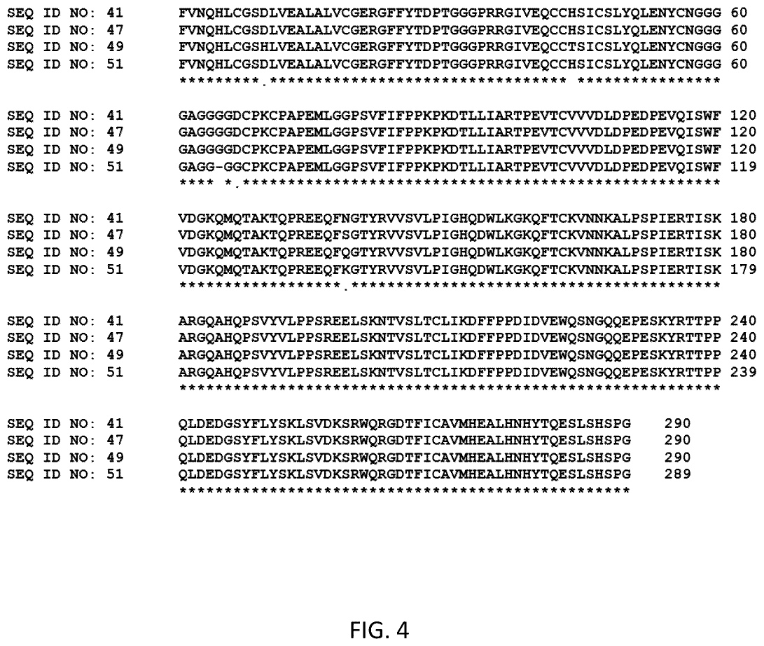

Therefore, cNg site mutations were made to the Fc fragment region of SEQ ID NO: 41 to reduce the binding affinity of the Fc fragment for Fc(gamma) receptors in vivo, as measured by binding in an in vitro human Fc(gamma)RI assay described in Example 12. Verification of the lack of glycan were performed using the LC-MS method of Example 7, but with omission of the PNGase F treatment step. The position of the cNg site in the insulin-Fc fusion protein of SEQ ID NO: 41 is cNg-NB139. Mutations to SEQ ID NO: 41 included SEQ ID NO: 47 comprising a mutation of cNg-NB139-S, SEQ ID NO: 49 comprising a mutation of cNg- NB139-Q together with reverting to the native B10H and A8T, and SEQ ID NO: 51 comprising removal of the aspartic acid at the N-terminus of the Fc fragment (des hinge D) a mutation of cNg-NB138-K (due to the des hinge D mutation, the cNg site for SEQ ID NO: 51 is NB138 instead of NB139). The full amino acid sequences of the cNg-mutated insulin-Fc fusion proteins are listed below (with the cNg position in bold) and the resulting sequence alignments are shown in FIG. 4 (Clustal Omega):

(SEQ ID NO: 47)
FVNQHLCGSDLVEALALVCGERGFFYTDPTGGGPRRGIVEQCCHSICSL
YQLENYCNGGGGAGGGGDCPKCPAPEMLGGPSVFIFPPKPKDTLLIART
PEVTCVVVDLDPEDPEVQISWFVDGKQMQTAKTQPREEQFSGTYRVVSV
LPIGHQDWLKGKQFTCKVNNKALPSPIERTISKARGQAHQPSVYVLPPS
REELSKNTVSLTCLIKDFFPPDIDVEWQSNGQQEPESKYRTTPPQLDED
GSYFLYSKLSVDKSRWQRGDTFICAVMHEALHNHYTQESLSHSPG
(SEQ ID NO: 49)
FVNQHLCGSHLVEALALVCGERGFFYTDPTGGGPRRGIVEQCCTSICSL
YQLENYCNGGGGAGGGGDCPKCPAPEMLGGPSVFIFPPKPKDTLLIART
PEVTCVVVDLDPEDPEVQISWFVDGKQMQTAKTQPREEQFQGTYRVVSV
LPIGHQDWLKGKQFTCKVNNKALPSPIERTISKARGQAHQPSVYVLPPS
REELSKNTVSLTCLIKDFFPPDIDVEWQSNGQQEPESKYRTTPPQLDED
GSYFLYSKLSVDKSRWQRGDTFICAVMHEALHNHYTQESLSHSPG
(SEQ ID NO: 51)
FVNQHLCGSDLVEALALVCGERGFFYTDPTGGGPRRGIVEQCCHSICSL
YQLENYCNGGGGAGGGGCPKCPAPEMLGGPSVFIFPPKPKDTLLIARTP
EVTCVVVDLDPEDPEVQISWFVDGKQMQTAKTQPREEQFKGTYRVVSVL
PIGHQDWLKGKQFTCKVNNKALPSPIERTISKARGQAHQPSVYVLPPSR
EELSKNTVSLTCLIKDFFPPDIDVEWQSNGQQEPESKYRTTPPQLDEDG
SYFLYSKLSVDKSRWQRGDTFICAVMHEALHNHYTQESLSHSPG

The insulin-Fc fusion proteins were manufactured in HEK293 cells according to Example 1 and purified using a Protein A column according to Example 3. The structures of the insulin-Fc fusion proteins were confirmed according to Example 5 by non-reducing and reducing CE-SDS, and the sequences were further identified by LC-MS with glycan removal according to Example 7. The % homodimer was measured by size-exclusion chromatography according to Example 9. As shown in Table 9, the homodimer titers of the insulin-Fc fusion proteins of SEQ ID NO: 47, SEQ ID NO: 49 and SEQ ID NO: 51 met the design goal for homodimer titer.

### Example 26: Screening of Canine Serum Containing Anti-Drug Antibodies and Identification of Potential Immunogenic Epitopes at the B10D and A8H Positions of the Insulin Polypeptide

It was hypothesized, therefore, that the insulin polypeptide of SEQ ID NO: 16 may unexpectedly contain specific epitopes (i.e., immunogenic “hot spots”) against which the dog's immune system is directed. Therefore, the binding specificity of the antibodies present in the serum samples described in Example 17 were evaluated according to the general procedure of Example 18. The analysis of the antibody-containing serum samples from the repeated dosing of the insulin-Fc fusion protein of SEQ ID NO: 41 (Example 24) against the coated insulin-Fc fusion protein library demonstrated that there were unexpectedly two primary “hot spots” present within the insulin polypeptide sequence of SEQ ID NO: 16: the aspartic acid mutation at the 10th position from the N-terminus of the B-chain (i.e., B10), and, separately, the histidine mutation at the 8th position from the N-terminal end of the A-chain (i.e., A8). The results suggest that insulin-Fc fusion proteins comprising insulin polypeptide amino acid compositions containing these two particular amino acid mutations are likely to be immunogenic in dogs and therefore likely to give rise to anti-drug antibodies that neutralize the bioactivity after repeated injections. The insulin-Fc fusion protein configurations of SEQ ID NO: 47 and SEQ ID NO: 51 however met the design requirements for IR binding IC50, while the insulin-Fc fusion protein configuration of SEQ ID NO: 49 (where the B10D and A8H mutations were restored to the native B10H and A8T) demonstrated an IR binding IC50 or >5000 nM, indicating that this insulin-Fc fusion protein configuration was highly unlikely to be bioactive in vivo. This indicated that the B10H and A8T mutations in these insulin-Fc fusion protein configurations appeared to be necessary to achieve in vivo bioactivity.

TABLE 9
Homodimer titers and IR binding for insulin-Fc fusion protein
configurations utilizing canine IgGB Fc with cNg mutations

| | | | | | IR |
|---|---|---|---|---|---|
| | | Protein | % | Homodimer | Binding, |
| | cNg | Yield | Homo- | Titer | IC50 |
| SEQ ID NO: | MUTATION | (mg/L) | dimer | (mg/L) | (nM) |
| SEQ ID NO: 47 | S | 76.7 | 98.3% | 75.4 | 145 |
| SEQ ID NO: 49 | Q | 21.4 | 98.7% | 21.1 | >5000 |
| SEQ ID NO: 51 | K | 45.4 | 98.1% | 44.5 | 147 |

### Example 27: Evaluation of In Vivo Bioactivity and Immunogenicity of Non-Glycosylated Insulin-Fc Fusion Protein Configurations of with cNg Mutations of the Canine IgGB Isotype Fc Fragment.

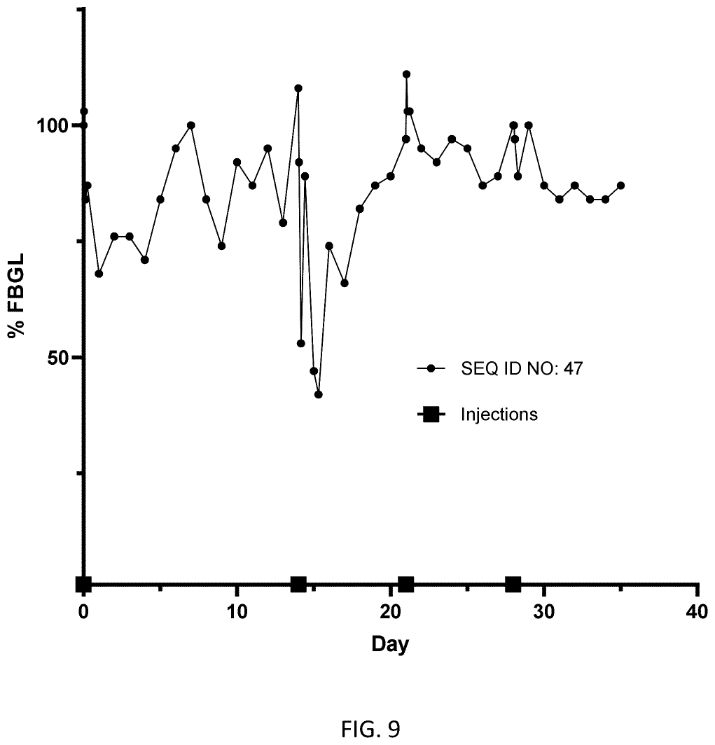

To determine if the insulin-Fc fusion protein of SEQ ID NO: 47, containing the cNg-S mutation, improved the repeated dose bioactivity performance in dogs, the compound was administered subcutaneously to N=1 dog on day 0, day 14, day 21 and on day 28 according to the procedure of Example 15. The % FBGL across these doses for SEQ ID NO: 47 is illustrated in FIG. 9. When the dog's % FBGL dropped too low, the dog was given food to raise the blood glucose to a safe level. The NAOC for the first injection measured over the first 7 days following the injection was 403% FBGL·days·kg/mg, showing that the insulin-Fc fusion protein of SEQ ID NO: 47 was satisfactorily bioactive in vivo. The NAOC and NAOCR were also measured for each subsequent dose according to the general procedure of Example 15, calculated from the time the dose was administered until just before the next dose was administered. The NAOC and the NAOCR shown in Table 10 illustrate that the insulin-Fc fusion protein of SEQ ID NO: 47 exhibited an NAOCR that decreased significantly on doses 2 and 3 of a three dose regimen. Therefore, the insulin-Fc fusion protein of SEQ ID NO: 47, containing the cNg-S mutation, was unable to demonstrate repeated dose bioactivity in dogs.

TABLE 10
NAOC per dose for repeated
doses of SEQ ID NO: 47

| Injection | NAOC | |
|---|---|---|
| Number of | (% FBGL · | |
| SEQ ID NO: 47 | days · kg/mg) | NAOCR |
| 1 | 403 | 1.0 |
| 2 | 508 | 0.3 |
| 3 | 128 | 0.32 |

To determine if the insulin-Fc fusion protein of SEQ ID NO: 49, containing the cNg-Q mutation, improved the repeated dose bioactivity performance in dogs, the compound was administered subcutaneously to N=1 dog on day 0 and on day 28 according to the procedure of Example 15. The % FBGL across these doses for SEQ ID NO: 49 is illustrated in FIG. 9. When the dog's % FBGL dropped too low, the dog was given food to raise the blood glucose to a safe level. The NAOC for the first injection measured over the first 7 days following the injection was 24.2% FBGL·days·kg/mg, showing that the insulin-Fc fusion protein of SEQ ID NO: 49 did not show satisfactory bioactive in vivo. The NAOC and NAOCR were also measured for the subsequent dose according to the general procedure of Example 15, calculated from the time the dose was administered until just before the next dose was administered. The NAOC and the NAOCR shown in Table 11 illustrate that the insulin-Fc fusion protein of SEQ ID NO: 49 exhibited 0 NAOC and NAOCR for the second dose. Therefore, the insulin-Fc fusion protein of SEQ ID NO: 49, containing the cNg-S mutation, was unable to demonstrate repeated dose bioactivity in dogs.

TABLE 11
NAOC per dose for repeated
doses of SEQ ID NO: 49

| Injection | NAOC | |
|---|---|---|
| Number of | (% FBGL · | |
| SEQ ID NO: 49 | days · kg/mg) | NAOCR |
| 1 | 403 | 1.0 |
| 2 | 0 | 0.0 |

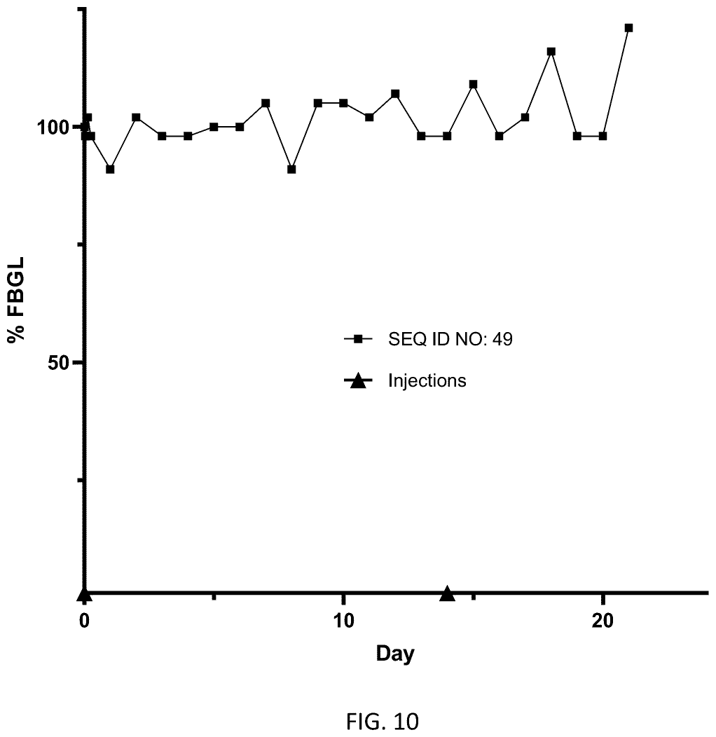

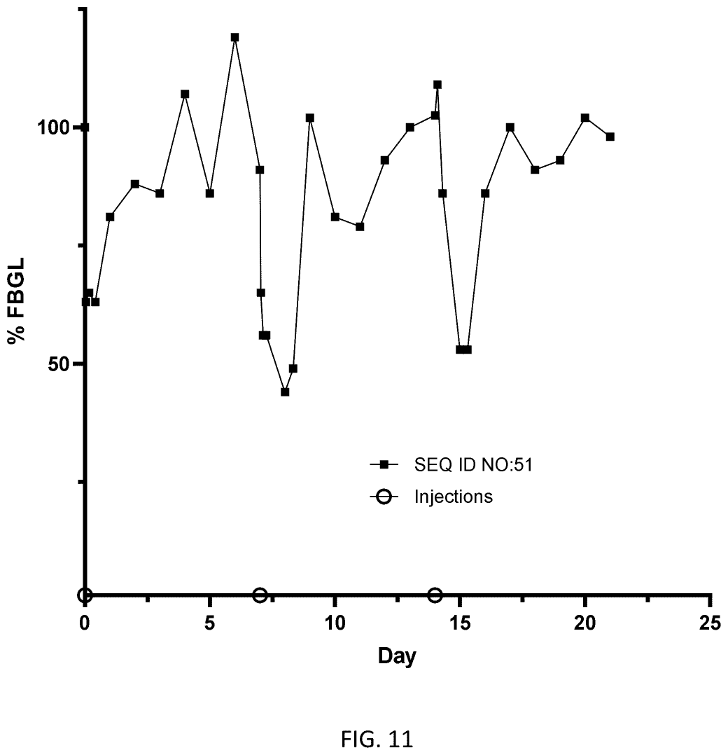

To determine if the insulin-Fc fusion protein of SEQ ID NO: 51, containing the cNg-K mutation together with the des hinge D omission (the Fc fragment of SEQ ID NO: 32) improved the repeated dose bioactivity performance in dogs, the compound was administered subcutaneously to N=1 dog on day 0, day 7, and day 14 according to the procedure of Example 15. The % FBGL across these doses for SEQ ID NO: 51 is illustrated in FIG. 10. When the dog's % FBGL dropped too low, the dog was given food to raise the blood glucose to a safe level. The NAOC for the first injection was 178% FBGL·days·kg/mg, showing that the insulin-Fc fusion protein of SEQ ID NO: 51 was satisfactorily bioactive in vivo. The NAOC and NAOCR were also measured for each subsequent dose according to the general procedure of Example 15, calculated from the time the dose was administered until just before the next dose was administered. The NAOC and the NAOCR shown in Table 12 illustrate that the insulin-Fc fusion protein configuration of SEQ ID NO: 51 maintains an NAOCR greater than 0.6 throughout the three doses. Therefore, unexpectedly, the insulin-Fc fusion protein of SEQ ID NO: 51, containing the cNg-K mutation with des hinge D, was the only non-glycosylated mutant of the insulin-Fc fusion protein of SEQ ID NO: 41 resulting in significantly improved repeated dose bioactivity in dogs.

TABLE 12
NAOC per dose for repeated
doses of SEQ ID NO: 51

| Injection | NAOC | |
|---|---|---|
| Number of | (% FBGL · | |
| SEQ ID NO: 51 | days · kg/mg) | NAOCR |
| 1 | 178 | 1.0 |
| 2 | 382 | 2.2 |
| 3 | 238 | 1.3 |

### Example 28: Evaluation of In Vivo Bioactivity and Immunogenicity of the Insulin-Fc Fusion Protein Configurations with the Fc Fragment of SEQ ID NO: 32

Based on the results from Example 27, insulin-Fc fusion protein configurations with the Fc fragment of SEQ ID NO: 32 and insulin polypeptide combinations based on the B-Chain, C-Chain and A-Chain sequences of Table 1 are evaluated for in vivo bioactivity and immunogenicity.

The insulin-Fc fusion protein configurations comprising insulin polypeptide combinations of Table 1 are manufactured in HEK293 cells according to Example 2 and purified using a Protein A column according to Example 4. The structures are confirmed according to Example 6 by non-reducing and reducing CE-SDS, and the sequences are further identified by LC-MS with glycan removal according to Example 8. The % homodimers are measured by size-exclusion chromatography according to Example 10. The in vivo bioactivity after a single dose of the insulin-Fc fusion protein configuration being tested is measured according to Example 14 and the in vivo bioactivity is measured after repeated doses according to Example 16.

It is expected that the insulin-Fc fusion protein configurations with the Fc fragment of SEQ ID NO: 32 and insulin polypeptide combinations based on the B-Chain, C-Chain and A-Chain sequences of Table 1 will exhibit acceptable homodimer titer and sustained bioactivity in vivo after repeated doses. That is, the NAOC and the NAOCR for each of the insulin-Fc fusion protein configurations based on the B-Chain, C-Chain and A-Chain sequences of Table I maintain an NAOCR greater than 0.6 over at least three doses.

### EQUIVALENTS

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, clements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, cach subgroup of the clements is also disclosed, and any element(s) can be removed from the group. It should be understood that, in general, where the disclosure, or aspects of the disclosure, is/are referred to as comprising particular clements and/or features, certain embodiments of the disclosure or aspects of the disclosure consist, or consist essentially of, such elements and/or features. For purposes of simplicity, those embodiments have not been specifically set forth in hacc verba herein. It is also noted that the terms “comprise(s),” “comprising,” “contain(s),” and “containing” are intended to be open and the use thereof permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

1. A method for lowering the blood glucose level of a dog, the method comprising administering a physiologically effective amount of a fusion protein or a pharmaceutical composition thereof to the dog, said fusion protein comprising an insulin polypeptide and an Fc fragment, wherein the insulin polypeptide and the Fc fragment are connected by a linker, wherein the Fc fragment comprises the following sequence:
CPKCPAPEMLGGPSVFIFPPKPKDTLLIARTPEVTCVVVDLDPEDPEVQISWFVDGKQMQTA KTQPREEQFKGTYRVVSVLPIGHQDWLKGKQFTCKVNNKALPSPIERTISKARGQAHQPSV YVLPPSREELSKNTVSLTCLIKDFFPPDIDVEWQSNGQQEPESKYRTTPPQLDEDGSYFLYSK LSVDKSRWQRGDTFICAVMHEALHNHYTQESLSHSPG (SEQ ID NO: 32)
and wherein the insulin polypeptide comprises the following sequence:
FVNQHLCGSDLVEALALVCGERGFFYTDPTGGGPRRGIVEQCCHSICSLYQLENYCN (SEQ ID NO: 16).

2. The method of claim 1, in which the dog is diagnosed with diabetes.

3. The method of claim 1, wherein the fusion protein is administered daily, twice weekly, or once weekly to the dog.

4. The method of claim 1, wherein the fusion protein is administered once weekly to the dog at a dose between 0.025 and 0.5 mg/kg/week.

5. The method of claim 1, wherein the fusion protein is administered to the dog subcutaneously.

6. The method of claim 5, wherein the NAOC after a first subcutaneous injection in the dog is greater than 150% FBGL·days·kg/mg.

7. The method of claim 5, wherein the ratio of the NAOC after the third weekly subcutaneous injection of the fusion protein in the dog to the NAOC after the first subcutaneous injection of the fusion protein in the dog is greater than 0.50.

8. The method of claim 1, wherein the serum half-life of the fusion protein in the blood or serum of the dog upon administration is longer than about 3 days.

9. The method of claim 1, wherein the time during which there is a statistically significant decrease in blood glucose level in the dog relative to a pre-dose level is longer than one of 2 hours, 6 hours, 9 hours, 12 hours, 18 hours, 1 day, 1.5 days, 2 days, 2.5 days, 3 days, 4 days, 5 days, 6 days, or 7 days.

10. The method of claim 1, wherein the fusion protein is present in the pharmaceutical composition at a concentration of about 3 mg/mL or greater.

11. The method of claim 1, wherein said fusion protein comprises the following sequence:
FVNQHLCGSDLVEALALVCGERGFFYTDPTGGGPRRGIVEQCCHSICSLYQLENYCNGGGG AGGGGCPKCPAPEMLGGPSVFIFPPKPKDTLLIARTPEVTCVVVDLDPEDPEVQISWFVDGK QMQTAKTQPREEQFKGTYRVVSVLPIGHQDWLKGKQFTCKVNNKALPSPIERTISKARGQA HQPSVYVLPPSREELSKNTVSLTCLIKDFFPPDIDVEWQSNGQQEPESKYRTTPPQLDEDGSY FLYSKLSVDKSRWQRGDTFICAVMHEALHNHYTQESLSHSPG (SEQ ID NO: 51).

12. The method of claim 1, wherein said fusion protein is a homodimer.
